SEQUENCE LISTING (186 sequences)

SEQ ID NO: 1 - protein, 126 aa, Homo sapiens
MQTGRTEDDLVPTAPSLGTKEGYLTKQGGLVKTWKTRWFTLHRNELKYFKDQMSPEPIRILDLTECSAVQFDYSQERVNCFCLVFPFRTFYLCAKTGVEADEWIKILRWKLSQIRKQLNQGEGTIR

SEQ ID NO: 2 - protein, 153 aa, Mus musculus
PFKIPEDDGNDLTHTFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 3 - protein, 118 aa, Homo sapiens
NPDREGWLLKLGGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASIS

SEQ ID NO: 4 - protein, 99 aa, Homo sapiens
SGSAREGWLFKWTNYIKGYQRRWFVLSNGLLSYYRSKAEMRHTCRGTINLATANITVEDSCNFIISNGGAQTYHLKASSEVERQRWVTALELAKAKAVK

SEQ ID NO: 5 - protein, 177 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 6 - protein, 98 aa, Homo sapiens
MEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDT

SEQ ID NO: 7 - protein, 122 aa, Homo sapiens
MSDNQSWNSSGSEEDPETESGPPVERCGVLSKWTNYIHGWQDRWVVLKNNALSYYKSEDETEYGCRGSICLSKAVITPHDFDECRFDISVNDSVWYLRAQDPDHRQQWIDAIEQHKTESGYG

SEQ ID NO: 8 - protein, 120 aa, Homo sapiens
TVMKEGWMVHYTSKDTLRKRHYWRLDSKCITLFQNDTGSRYYKEIPLSEILSLEPVKTSALIPNGANPHCFEITTANVVYYVGENVVNPSSPSPNNSVLTSGVGADVARMWEIAIQHALM

SEQ ID NO: 9 - protein, 101 aa, Homo sapiens
RIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVAS

SEQ ID NO: 10 - protein, 97 aa, Homo sapiens
DVLKQGYMMKKGHRRKNWTERWFVLKPNIISYYVSEDLKDKKGDILLDENCCVESLPDKDGKKCLFLVKCFDKTFEISASDKKKKQEWIQAIHSTIH

SEQ ID NO: 11 - protein, 113 aa, Homo sapiens
DMLSSHHYKSFKVSMIHRLRFTTDVQLGISGDKVEIDPVTNQKASTKFWIKQKPISIDSDLLCACDLAEEKSPSHAIFKLTYLSNHDYKHLYFESDAATVNEIVLKVNYILES

SEQ ID NO: 12 - protein, 562 aa, unidentified
  REGION 1..562: note = Description of Unknown:Baboon endogenous virus sequence
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLVQFVKDRISVVQALVLTQQYHQLKPLEYEP

SEQ ID NO: 13 - protein, 546 aa, synthetic construct
  REGION 1..546: note = Description of Artificial Sequence: Syntheticpolypeptide
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 14 - protein, 504 aa, synthetic construct
  REGION 1..504: note = Description of Artificial Sequence: Syntheticpolypeptide
MVPQVLLFVLLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASNGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDDHVLIPEMQSSLLQQHMELLESSVIPLMHPLADPSTVFKEGDEAEDFVEVHLPKNPIELVEGWFSSWKSSIASFFFIIGLIIGLFLVLRVGIHLCIKLKHTKKRQIYTDIEMNRLGK

SEQ ID NO: 15 - protein, 504 aa, synthetic construct
  REGION 1..504: note = Description of Artificial Sequence: Syntheticpolypeptide
MVPQVLLFVLLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRVFPGGKCSGITVSSTYCSTNHDYTIWMPENPRPRTPCDIFTNSRGKRASNGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDDHVLIPEMQSSLLQQHMELLESSVIPLMHPLADPSTVFKEGDEAEDFVEVHLEKNPIELVEGWFSSWKSSIASFFFIIGLIIGLFLVLRVGIHLCIKLKHTKKRQIYTDIEMNRLGK

SEQ ID NO: 16 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 17 - protein, 665 aa, Murine leukemia virus
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 18 - protein, 645 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVLTQQYHQLKPIEYEP

SEQ ID NO: 19 - protein, 1710 aa, synthetic construct
  REGION 1..1710: note = Description of Artificial Sequence: Syntheticpolypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 20 - protein, 198 aa, Homo sapiens [sequence omitted]

SEQ ID NO: 21 - protein, 191 aa, synthetic construct
  REGION 1..191: note = Description of Artificial Sequence: Syntheticpolypeptide
LMDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 22 - protein, 175 aa, synthetic construct
  REGION 1..175: note = Description of Artificial Sequence: Syntheticpolypeptide
MDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPL

SEQ ID NO: 23 - protein, 229 aa, Rattus norvegicus [sequence omitted]

SEQ ID NO: 24 - protein, 397 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFRPWKGLEIISRRTQRRLRRIKESWGLQDLVNDFGNLQLGPPMSN

SEQ ID NO: 25 - protein, 199 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRR

SEQ ID NO: 26 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 27 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 28 - protein, 186 aa, Homo sapiens
PPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 29 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 30 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 31 - protein, 189 aa, Homo sapiens
KEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 32 - protein, 383 aa, Escherichia coli
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 33 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 34 - protein, 1419 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGGGGSGKRTADGSEFEPKKKRKVSSGGDYKDHDGDYKDHDIDYKDDDDK

SEQ ID NO: 35 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 36 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 37 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 38 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 39 - protein, 1228 aa, unidentified
  REGION 1..1228: note = Description of Unknown:Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 40 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 41 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIAHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIANYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 42 - protein, 175 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKELNIQ

SEQ ID NO: 43 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 44 - protein, 92 aa, Homo sapiens
KMGPVDKRKGLFARRRQLLLTEGPHLYYVDPVNKVLKGEIPWSQELRPEAKNFKTFFVHTPNRTYYLMDPSGNAHKWCRKIQEVWRQRYQSH

SEQ ID NO: 45 - protein, 38 aa, Human alphaherpesvirus 1
PTDALDDFDLDMLPADALDDFDLDMLPADALDDFDLDM

SEQ ID NO: 46 - protein, 53 aa, synthetic construct
  REGION 1..53: note = Description of Artificial Sequence: Syntheticpolypeptide
GRADALDDFDLDMLGSDALDDFDLDMLGSDALDDFDLDMLGSDALDDFDLDML

SEQ ID NO: 47 - protein, 261 aa, Homo sapiens
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 48 - protein, 190 aa, Human gammaherpesvirus 8
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 49 - protein, 72 aa, Homo sapiens
MDAKSLTAWSRTLVTFKDVFVDFTREEWKLLDTAQQIVYRNVMLENYKNLVSLGYQLTKPDVILRLEKGEEP

SEQ ID NO: 50 - protein, 289 aa, Homo sapiens
EASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 51 - protein, 718 aa, Homo sapiens
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 52 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 53 - protein, 511 aa, Indiana vesiculovirus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 54 - protein, 512 aa, unidentified
  REGION 1..512: note = Description of Unknown:Baculovirus envelope glycoprotein GP64 sequence
MVSAIVLYVLLAAAAHSAFAAEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILFLYCMIRNRNRQY

SEQ ID NO: 55 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 56 - protein, 564 aa, unidentified
  REGION 1..564: note = Description of Unknown:Endogenous feline virus sequence
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLATNPLWTGLQGFLPYLLPLLGPLLTLLLILTIGPCVFSRLMAFINDRLNVVHAMVLAQQYQALKAEEEAQD

SEQ ID NO: 57 - protein, 229 aa, Homo sapiens
MSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 58 - protein, 238 aa, Homo sapiens
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 59 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 60 - protein, 10 aa, Homo sapiens
EVTELTREGE

SEQ ID NO: 61 - protein, 21 aa, Homo sapiens
GNYTCEVTELTREGETIIELK

SEQ ID NO: 62 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 63 - protein, 397 aa, Adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 64 - protein, 621 aa, Adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 65 - protein, 735 aa, Adeno-associated virus 2 [sequence omitted]

SEQ ID NO: 66 - protein, 597 aa, Adeno-associated virus 2
APGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 67 - protein, 533 aa, Adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 68 - protein, 252 aa, synthetic construct
  REGION 1..252: note = Description of Artificial Sequence: Syntheticpolypeptide
EQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 69 - protein, 110 aa, synthetic construct
  REGION 1..110: note = Description of Artificial Sequence: Syntheticpolypeptide
FKCEHCRILFLDHVMFTIHMGCHGFRDPFKCNMCGEKCDGPVGLFVHMARNAHGEKPFYCEHCEITFRDVVMYSLHKGYHGFRDPFECNICGYHSQDRYEFSSHIVRGEH

SEQ ID NO: 70 - protein, 110 aa, synthetic construct
  REGION 1..110: note = Description of Artificial Sequence: Syntheticpolypeptide
HHCQHCDMYFADNILYTIHMGCHSCDDVFKCNMCGEKCDGPVGLFVHMARNAHGEKPTKCVHCGIVFLDEVMYALHMSCHGFRDPFECNICGYHSQDRYEFSSHIVRGEH

SEQ ID NO: 71 - protein, 110 aa, synthetic construct
  REGION 1..110: note = Description of Artificial Sequence: Syntheticpolypeptide
MGRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 72 - protein, 111 aa, synthetic construct
  REGION 1..111: note = Description of Artificial Sequence: Syntheticpolypeptide
MASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 73 - protein, 97 aa, synthetic construct
  REGION 1..97: note = Description of Artificial Sequence: Syntheticpolypeptide
MGSRILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 74 - protein, 108 aa, synthetic construct
  REGION 1..108: note = Description of Artificial Sequence: Syntheticpolypeptide
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 75 - protein, 93 aa, synthetic construct
  REGION 1..93: note = Description of Artificial Sequence: Syntheticpolypeptide
QGMLEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 76 - protein, 277 aa, synthetic construct
  REGION 1..277: note = Description of Artificial Sequence: Syntheticpolypeptide
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 77 - protein, 237 aa, synthetic construct
  REGION 1..237: note = Description of Artificial Sequence: Syntheticpolypeptide
EELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGS

SEQ ID NO: 78 - protein, 573 aa, synthetic construct
  REGION 1..573: note = Description of Artificial Sequence: Syntheticpolypeptide
EELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKDYHLENEVARLKKGSGSGEELLSKNYHLENEVARLKKGS

SEQ ID NO: 79 - protein, 114 aa, synthetic construct
  REGION 1..114: note = Description of Artificial Sequence: Syntheticpolypeptide
VQLVESGGALVQPGGSLRLSCAASGFPVNRYSMRWYRQAPGKEREWVAGMSSAGDRSSYEDSVKGRFTISRDDARNTVYLQMNSLKPEDTAVYYSNVNVGFEYWGQGTQVTVSS

SEQ ID NO: 80 - protein, 102 aa, Nostoc commune
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 81 - protein, 39 aa, Nostoc commune
MIKIATRKYLGKQNVYDIGVERDHNFALKNGFIASNCFN

SEQ ID NO: 82 - protein, 101 aa, synthetic construct
  REGION 1..101: note = Description of Artificial Sequence: Syntheticpolypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 83 - protein, 36 aa, synthetic construct
  REGION 1..36: note = Description of Artificial Sequence: Syntheticpolypeptide
MVKIISRKSLGTQNVYDIGVEKDHNFLLKNGLVASN

SEQ ID NO: 84 - protein, 184 aa, Saccharomyces cerevisiae
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILY

SEQ ID NO: 85 - protein, 65 aa, Saccharomyces cerevisiae
VLLNVLSKCAGSKKFRPAPAAAFARECRGFYFELQELKEDDYYGITLSDDSDHQFLLANQVVVHN

SEQ ID NO: 86 - protein, 123 aa, Synechocystis sp.
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 87 - protein, 36 aa, Synechocystis sp.
MVKVIGRRSLGVQRIFDIGLPQDHNFLLANGAIAAN

SEQ ID NO: 88 - protein, 14 aa, synthetic construct
  REGION 1..14: note = Description of Artificial Sequence: Syntheticpeptide
VPTIVMVDAYKRYK

SEQ ID NO: 89 - protein, 119 aa, synthetic construct
  REGION 1..119: note = Description of Artificial Sequence: Syntheticpolypeptide
MVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 90 - protein, 130 aa, unidentified
  REGION 1..130: note = Description of Unknown:Enterobacteria phage MS2 sequence
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 91 - protein, 130 aa, unidentified
  REGION 1..130: note = Description of Unknown:Enterobacteria phage MS2 sequence
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 92 - protein, 130 aa, unidentified
  REGION 1..130: note = Description of Unknown:Enterobacteria phage MS2 sequence
MASNFTQFVLVDNGGTGDVTVAPSNFANGIAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 93 - protein, 121 aa, unidentified
  REGION 1..121: note = Description of Unknown:Pseudomonas phage PP7 sequence
KTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRS

SEQ ID NO: 94 - protein, 62 aa, Escherichia virus Mu
MKSIRCKNCNKLLFKADSFDHIEIRCPRCKRHIIMLNACEHPTEKHCGKREKITHSDETVRY

SEQ ID NO: 95 - protein, 179 aa, synthetic construct
  REGION 1..179: note = Description of Artificial Sequence: Syntheticpolypeptide
STRPGERPFQCRICMRNFSIPNHLARHTRTHTGEKPFQCRICMRNFSQSAHLKRHLRTHTGEKPFQCRICMRNFSQDVSLVRHLKTHLRQKDGERPFQCRICMRNFSSAQALARHTRTHTGEKPFQCRICMRNFSQGGNLTRHLRTHTGEKPFQCRICMRNFSQHPNLTRHLKTHLRGS

SEQ ID NO: 96 - protein, 178 aa, synthetic construct
  REGION 1..178: note = Description of Artificial Sequence: Syntheticpolypeptide
SRPGERPFQCRICMRNFSTMAVLRRHTRTHTGEKPFQCRICMRNFSRREVLENHLRTHTGEKPFQCRICMRNFSQTVNLDRHLKTHLRQKDGERPFQCRICMRNFSKKDHLHRHTRTHTGEKPFQCRICMRNFSQRPHLTNHLRTHTGEKPFQCRICMRNFSVGASLKRHLKTHLRGS

SEQ ID NO: 97 - protein, 90 aa, synthetic construct
  REGION 1..90: note = Description of Artificial Sequence: Syntheticpolypeptide
SRPGERPFQCRICMRNFSDKTKLRVHTRTHTGEKPFQCRICMRNFSVRHNLTRHLRTHTGEKPFQCRICMRNFSQSTSLQRHLKTHLRGF

SEQ ID NO: 98 - protein, 176 aa, synthetic construct
  REGION 1..176: note = Description of Artificial Sequence: Syntheticpolypeptide
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 99 - protein, 86 aa, synthetic construct
  REGION 1..86: note = Description of Artificial Sequence: Syntheticpolypeptide
YACPVESCDRRFSRSDELTRHIRIHTGQKPFQCRICMRNFSRSDHLTTHIRTHTGEKPFACDICGRKFARSDERKRHTKIHLRQKD

SEQ ID NO: 100 - protein, 86 aa, synthetic construct
  REGION 1..86: note = Description of Artificial Sequence: Syntheticpolypeptide
YACPVESCDRRFSQSHDLTKHIRIHTGQKPFQCRICMRNFSDSSKLSRHIRTHTGEKPFACDICGRKFARLDNRTAHTKIHLRQKD

SEQ ID NO: 101 - protein, 176 aa, synthetic construct
  REGION 1..176: note = Description of Artificial Sequence: Syntheticpolypeptide
YACPVESCDRRFSRSDELTRHIRIHTGQKPFQCRICMRNFSRSDHLTTHIRTHTGEKPFACDICGRKFARSDERKRHTKIHLRQKDGERPYACPVESCDRRFSQSHDLTKHIRIHTGQKPFQCRICMRNFSDSSKLSRHIRTHTGEKPFACDICGRKFARLDNRTAHTKIHLRQKD

SEQ ID NO: 102 - protein, 179 aa, synthetic construct
  REGION 1..179: note = Description of Artificial Sequence: Syntheticpolypeptide
YACPVESCDRRFSRSDELTRHIRIHTGQKPFQCRICMRNFSRSDHLTTHIRTHTGEKPFACDICGRKFARSDERKRHTKIHLRQKDGGGSERPYACPVESCDRRFSQSHDLTKHIRIHTGQKPFQCRICMRNFSDSSKLSRHIRTHTGEKPFACDICGRKFARLDNRTAHTKIHLRQKD

SEQ ID NO: 103 - protein, 286 aa, synthetic construct
  REGION 1..286: note = Description of Artificial Sequence: Syntheticpolypeptide
SRPGERPFQCRICMRNFSTRQNLDTHTRTHTGEKPFQCRICMRNFSRRDTLERHLRTHTGEKPFQCRICMRNFSRPDALPRHLKTHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 104 - protein, 286 aa, synthetic construct
  REGION 1..286: note = Description of Artificial Sequence: Syntheticpolypeptide
SRPGERPFQCRICMRNFSSPSKLIRHTRTHTGEKPFQCRICMRNFSDGSNLARHLRTHTGEKPFQCRICMRNFSRVDNLPRHLKTHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 105 - protein, 353 aa, synthetic construct
  REGION 1..353: note = Description of Artificial Sequence: Syntheticpolypeptide
MDYKDHDGDYKDHDIDYKDDDDKMAPKKKRKVGIHGVPAAMAERPFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 106 - protein, 352 aa, synthetic construct
  REGION 1..352: note = Description of Artificial Sequence: Syntheticpolypeptide
MDYKDHDGDYKDHDIDYKDDDDKMAPKKKRKVGIHGVPAAMAERPFQCRICMRNFSRSDNLSVHIRTHTGEKPFACDICGRKFAQKINLQVHTKIHTGEKPFQCRICMRNFSRSDVLSEHIRTHTGEKPFACDICGRKFAQRNHRTTHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 107 - protein, 196 aa, synthetic construct
  REGION 1..196: note = Description of Artificial Sequence: Syntheticpolypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 108 - protein, 199 aa, synthetic construct
  REGION 1..199: note = Description of Artificial Sequence: Syntheticpolypeptide
VHDHKLELAKLIRNYETNRKECLNSRYNETLLRSDYLDPFFELLGWDIKNKAGKPTNEREVVLEEALKASASEHSKKPDYTFRLFSERKFFLEAKKPSVHIESDNETAKQVRRYGFTAKLKISVLSNFEYLVIYDTSVKVDGDDTFNKARIKKYHYTEYETHFDEICDLLGRESVYSGNFDKEWLSIENKINHFSVDTL

SEQ ID NO: 109 - protein, 119 aa, synthetic construct
  REGION 1..119: note = Description of Artificial Sequence: Syntheticpolypeptide
YNETLLRSDYLDPFFELLGWDIKNKAGKPTNEREVVLEEALKASASEHSKKPDYTFRLFSERKFFLEAKKPSVHIESDNETAKQVRRYGFTAKLKISVLSNFEYLVIYDTSVKVDGDDT

SEQ ID NO: 110 - protein, 165 aa, Escherichia coli
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDTQN

SEQ ID NO: 111 - protein, 165 aa, Escherichia coli
MLKPEMIEKLNEQMNLELYSSLLYQQMSAWCSYLTFEGAAAFLRRHAQEEMTHMQRLFDYLTDIGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDTQN

SEQ ID NO: 112 - protein, 389 aa, synthetic construct
  REGION 1..389: note = Description of Artificial Sequence: Syntheticpolypeptide
MTSQIRQNYSTEVEAAVNRLVNLHLRASYTYLSLGFFFDRDDVALEGVGHFFRELAEEKREGAERLLEFQNDRGGRALFQDVQKPSQDEWGKTQEAMEAALAMEKNLNQALLDLHALGSARADPHLCDFLESHYLDKEVKLIKKMGNHLTNLRRVAGPQPAQTGAPQGSLGEYLFERLTLKHDARGGGGSDYKDDDDKGGGGSRVMTTASPSQVRQNYHQDAEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDESTR

SEQ ID NO: 113 - protein, 378 aa, synthetic construct
  REGION 1..378: note = Description of Artificial Sequence: Syntheticpolypeptide
SQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHDARGGGGSDYKDDDDKGGGGSRVMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 114 - protein, 612 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSAAVKPEEEEERDMKKSRGFDERELFSTAESSSSSSVFFVSQSCSLASEGKNLEGIQDSSDQITTSLGKNGCK

SEQ ID NO: 115 - protein, 170 aa, Arabidopsis thaliana
MNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYI

SEQ ID NO: 116 - protein, 144 aa, synthetic construct
  REGION 1..144: note = Description of Artificial Sequence: Syntheticpolypeptide
SLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKEL

SEQ ID NO: 117 - protein, 740 aa, Homo sapiens
DIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 118 - protein, 740 aa, Homo sapiens
DIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 119 - protein, 420 aa, Homo sapiens
VLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 120 - protein, 420 aa, synthetic construct
  REGION 1..420: note = Description of Artificial Sequence: Syntheticpolypeptide
VLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 121 - protein, 584 aa, synthetic construct
  REGION 1..584: note = Description of Artificial Sequence: Syntheticpolypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSLPPLERLT

SEQ ID NO: 122 - protein, 585 aa, synthetic construct
  REGION 1..585: note = Description of Artificial Sequence: Syntheticpolypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPVGSGSGSLPPLERLTL

SEQ ID NO: 123 - protein, 972 aa, Ruminococcus flavefaciens
EASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSG

SEQ ID NO: 124 - protein, 991 aa, synthetic construct
  REGION 1..991: note = Description of Artificial Sequence: Syntheticpolypeptide
EASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

SEQ ID NO: 125 - protein, 1094 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQ

SEQ ID NO: 126 - protein, 25 aa, synthetic construct
  REGION 1..25: note = Description of Artificial Sequence: Syntheticpeptide
IWLTALKFLGKHAAKHEAKQQLSKL

SEQ ID NO: 127 - protein, 75 aa, synthetic construct
  REGION 1..75: note = Description of Artificial Sequence: Syntheticpolypeptide
IWLTALKFLGKHAAKHEAKQQLSKLNAVGQDTQEVIVVPHSLPFKVVVISAILALVVLTIISLIILIMLWQKKPR

SEQ ID NO: 128 - protein, 30 aa, synthetic construct
  REGION 1..30: note = Description of Artificial Sequence: Syntheticpolypeptide
WEAKLAKALAKALAKHLAKALAKALKACEA

SEQ ID NO: 129 - protein, 80 aa, synthetic construct
  REGION 1..80: note = Description of Artificial Sequence: Syntheticpolypeptide
WEAKLAKALAKALAKHLAKALAKALKACEANAVGQDTQEVIVVPHSLPFKVVVISAILALVVLTIISLIILIMLWQKKPR

SEQ ID NO: 130 - protein, 26 aa, synthetic construct
  REGION 1..26: note = Description of Artificial Sequence: Syntheticpeptide
KKALLHAALAHLLALAHHLLALLKKA

SEQ ID NO: 131 - protein, 76 aa, synthetic construct
  REGION 1..76: note = Description of Artificial Sequence: Syntheticpolypeptide
KKALLHAALAHLLALAHHLLALLKKANAVGQDTQEVIVVPHSLPFKVVVISAILALVVLTIISLIILIMLWQKKPR

SEQ ID NO: 132 - protein, 50 aa, synthetic construct
  REGION 1..50: note = Description of Artificial Sequence: Syntheticpolypeptide
NAVGQDTQEVIVVPHSLPFKVVVISAILALVVLTIISLIILIMLWQKKPR

SEQ ID NO: 133 - protein, 34 aa, Lactococcus lactis
ITSISLCTPGCKTGALMGCNMKTATCHCSIHVSK

SEQ ID NO: 134 - protein, 34 aa, Lactococcus lactis
ITSISLCTPGCKTGALMGCNMKTATCNCSIHVSK

SEQ ID NO: 135 - protein, 34 aa, Lactococcus lactis
ITSISLCTPGCKTGALMGCNMKTATCNCSVHVSK

SEQ ID NO: 136 - protein, 34 aa, Lactococcus lactis
ITSISLCTPGCKTGVLMGCNLKTATCNCSVHVSK

SEQ ID NO: 137 - protein, 34 aa, Streptococcus hyointestinalis
FTSISMCTPGCKTGALMTCNYKTATCHCSIKVSK

SEQ ID NO: 138 - protein, 31 aa, Streptococcus uberis
ITSKSLCTPGCKTGILMTCPLKTATCGCHFG

SEQ ID NO: 139 - protein, 31 aa, Streptococcus uberis
VTSKSLCTPGCKTGILMTCPLKTATCGCHFG

SEQ ID NO: 140 - protein, 31 aa, Streptococcus gallolyticus
VTSKSLCTPGCKTGILMTCAIKTATCGCHFG

SEQ ID NO: 141 - protein, 34 aa, Lactococcus lactis
ITSISLCTPGCKTGALMGCNMKTATCHCAIHVSK

SEQ ID NO: 142 - protein, 34 aa, Lactococcus lactis
ITSISLCTPGCKTGALMGCNMKTATCHCDIHVSK

SEQ ID NO: 143 - protein, 34 aa, Lactococcus lactis
ITSISLCTPGCKTGALMGCNMKTATCHCEIHVSK

SEQ ID NO: 144 - protein, 34 aa, Lactococcus lactis
ITSISLCTPGCKTGALMGCNMKTATCHCGIHVSK

SEQ ID NO: 145 - protein, 34 aa, Lactococcus lactis
ITSISLCTPGCKTGALMGCNMTTATCHCSIHVSK

SEQ ID NO: 146 - protein, 34 aa, Lactococcus lactis
ITSISLCTPGCKTGALMGCPMKTATCHCSIHVSK

SEQ ID NO: 147 - protein, 34 aa, Lactococcus lactis
ITSISLCTPGCKTGALMGCNVKTATCHCSIHVSK

SEQ ID NO: 148 - protein, 34 aa, Lactococcus lactis
ITSISLCTPGCKTGALMGCNMSTATCHCSIHVSK

SEQ ID NO: 149 - protein, 34 aa, Lactococcus lactis
ITSISLCTPGCKTGALMGCKMKTATCNCSIHVSK

SEQ ID NO: 150 - protein, 34 aa, Lactococcus lactis
ITSISLCTPGCKTGALMGCNKKTATCNCSIHVSK

SEQ ID NO: 151 - RNA, 143 nt, synthetic construct
  misc_feature 1..143: note = Description of Artificial Sequence: Syntheticpolynucleotide
  source 1..143: mol_type = other RNA
gttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 152 - RNA, 199 nt, synthetic construct
  misc_feature 1..199: note = Description of Artificial Sequence: Syntheticpolynucleotide
  source 1..199: mol_type = other RNA
gttttagagctaggccaacatgaggatcacccatgtctgcagggcctagcaagttaaaataaggctagtccgttatcaacttggccaacatgaggatcacccatgtctgcagggccaagtggcaccgagtcggtgcgggagcacatgaggatcacccatgtgcgactcccacagtcactggggagtcttcccttttttt

SEQ ID NO: 153 - RNA, 149 nt, synthetic construct
  misc_feature 1..149: note = Description of Artificial Sequence: Syntheticpolynucleotide
  source 1..149: mol_type = other RNA
gtttaagagctatgctggaaacagcatagcaagtttaaataaggctagtccgttatcaacttgaaaaagtggcaccgagtcggtgcgggagcacatgaggatcacccatgtgcgactcccacagtcactggggagtcttcccttttttt

SEQ ID NO: 154 - RNA, 186 nt, synthetic construct
  misc_feature 1..186: note = Description of Artificial Sequence: Syntheticpolynucleotide
  source 1..186: mol_type = other RNA
ttctagatcatcgaaacatgaggatcacccatatctgcagtcgacatcgaaacatgaggatcacccatgtctgcagtcgacatcgaaacatgaggatcacccatgtctgcagtcgacatcgaaacatgaggatcacccatgtctgcagtcgacatcgaaatcgataagcttcagatcagatcctag

SEQ ID NO: 155 - RNA, 19 nt, synthetic construct
  misc_feature 1..19: note = Description of Artificial Sequence: Syntheticoligonucleotide
  source 1..19: mol_type = other RNA
acatgaggatcacccatgt

SEQ ID NO: 156 - RNA, 19 nt, synthetic construct
  misc_feature 1..19: note = Description of Artificial Sequence: Syntheticoligonucleotide
  source 1..19: mol_type = other RNA
acatgaggatcacccatat

SEQ ID NO: 157 - RNA, 14 nt, synthetic construct
  misc_feature 1..14: note = Description of Artificial Sequence: Syntheticoligonucleotide
  source 1..14: mol_type = other RNA
ccacagtcactggg

SEQ ID NO: 158 - RNA, 89 nt, synthetic construct
  misc_feature 1..89: note = Description of Artificial Sequence: Syntheticoligonucleotide
  source 1..89: mol_type = other RNA
acatgaggatcacccatgtctgcagggcctagcaagttaaaataaggctagtccgttatcaacttggccaacatgaggatcacccatgt

SEQ ID NO: 159 - RNA, 141 nt, synthetic construct
  misc_feature 1..141: note = Description of Artificial Sequence: Syntheticpolynucleotide
  source 1..141: mol_type = other RNA
gttttagagctaggccggagcagacgatatggcgtcgctccggcctagcaagttaaaataaggctagtccgttatcaacttggccggagcagacgatatggcgtcgctccggccaagtggcaccgagtcggtgcttttttt

SEQ ID NO: 160 - RNA, 32 nt, synthetic construct
  misc_feature 1..32: note = Description of Artificial Sequence: Syntheticoligonucleotide
  source 1..32: mol_type = other RNA
gccggagcagacgatatggcgtcgctccggcc

SEQ ID NO: 161 - RNA, 112 nt, synthetic construct
  misc_feature 1..112: note = Description of Artificial Sequence: Syntheticpolynucleotide
  source 1..112: mol_type = other RNA
gtttaagagctatgctggaaacagcatagcaagtttaaataaggctagtccgttatcaacttgaaaaagtggcaccgagtcggtgcctgaatgcctgcgagcatcttttttt

SEQ ID NO: 162 - RNA, 19 nt, synthetic construct
  misc_feature 1..19: note = Description of Artificial Sequence: Syntheticoligonucleotide
  source 1..19: mol_type = other RNA
ctgaatgcctgcgagcatc

SEQ ID NO: 163 - RNA, 448 nt, synthetic construct
  misc_feature 1..448: note = Description of Artificial Sequence: Syntheticpolynucleotide
  source 1..448: mol_type = other RNA
agtgacagtggagattgtacagttttttcctcgatttgtcaggattttttttttttgacggagtttaacttcttgtctcccaggtaggaagtgcagtggcgtaatctcggctcactacaacctccacctcctgggttcaagcgtttctcctgcctcagctttccgagtagctgggattacaggcgcctgccaccatgccctgctgacttttgtatttttagtagagacggggtttcaccatgttggccaggctggtcttgaactcctgaccgcaggcgattggcctgcctcggcctcccaaagtgctgagattacaggcgtgagccaccacccccggcctcaggagcgttctgatagtgcctcgatgtgctgcctcctataaagtgttagcagcacagatcactttttgtaaaggtacgtactaatgactttttttttatacttcagg

SEQ ID NO: 164 - RNA, 796 nt, synthetic construct
  misc_feature 1..796: note = Description of Artificial Sequence: Syntheticpolynucleotide
  source 1..796: mol_type = other RNA
taagaagcaaggtttcatttaggggaagggaaatgattcaggacgagagtctttgtgctgctgagtgcctgtgatgaagaagcatgttagtcctgggcaacgtagcgagaccccatctctacaaaaaatagaaaaattagccaggtatagtggcgcacacctgtgattccagctacgcaggaggctgaggtgggaggattgcttgagcccaggaggttgaggctgcagtgagctgtaatcatgccactactccaacctgggcaacacagcaaggaccctgtctcaaaagctacttacagaaaagaattaggctcggcacggtagctcacacctgtaatcccagcactttgggaggctgaggcgggcagatcacttgaggtcaggagtttgagaccagcctggccaacatggtgaaaccttgtctctactaaaaatatgaaaattagccaggcatggtggcacattcctgtaatcccagctactcgggaggctgaggcaggagaatcacttgaacccaggaggtggaggttgcagtaagccgagatcgtaccactgtgctctagccttggtgacagagcgagactgtcttaaaaaaaaaaaaaaaaaaaaaagaattaattaaaaatttaaaaaaaaatgaaaaaaagctgcatgcttgttttttgtttttagttattctacattgttgtcattattaccaaatattggggaaaatacaacttacagaccaatctcaggagttaaatgttactacgaaggcaaatgaactatgcgtaatgaacctggtaggcattag

SEQ ID NO: 165 - RNA, 133 nt, synthetic construct
  misc_feature 1..133: note = Description of Artificial Sequence: Syntheticpolynucleotide
  source 1..133: mol_type = other RNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 166 - DNA, 18 bp, synthetic construct
  misc_feature 1..18: note = Description of Artificial Sequence: Syntheticoligonucleotide
  source 1..18: mol_type = other DNA
gaagaagctgcaggaggt

SEQ ID NO: 167 - DNA, 18 bp, synthetic construct
  misc_feature 1..18: note = Description of Artificial Sequence: Syntheticoligonucleotide
  source 1..18: mol_type = other DNA
gctggaggggaagtggtc

SEQ ID NO: 168 - DNA, 36 bp, synthetic construct
  misc_feature 1..36: note = Description of Artificial Sequence: Syntheticoligonucleotide
  source 1..36: mol_type = other DNA
gaagaagctgcaggaggtgctggaggggaagtggtc

SEQ ID NO: 169 - DNA, 346 bp, synthetic construct
  misc_feature 1..346: note = Description of Artificial Sequence: Syntheticpolynucleotide
  source 1..346: mol_type = other DNA
tgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtccggatcttgaagaagctgcaggaggtgctggaggggaagtggtcc

SEQ ID NO: 170 - skipped

SEQ ID NO: 171 - DNA, 20 bp, synthetic construct
  misc_feature 1..20: note = Description of Artificial Sequence: Syntheticoligonucleotide
  source 1..20: mol_type = other DNA
cggcgtagccgatgtcgcgc

SEQ ID NO: 172-173 - skipped (2 entries)

SEQ ID NO: 174 - DNA, 18 bp, synthetic construct
  misc_feature 1..18: note = Description of Artificial Sequence: Syntheticoligonucleotide
  source 1..18: mol_type = other DNA
aagggttcagcgtgggcg

SEQ ID NO: 175 - DNA, 19 bp, synthetic construct
  misc_feature 1..19: note = Description of Artificial Sequence: Syntheticoligonucleotide
  source 1..19: mol_type = other DNA
aagggttcaggcgtgggcg

SEQ ID NO: 176 - DNA, 20 bp, synthetic construct
  misc_feature 1..20: note = Description of Artificial Sequence: Syntheticoligonucleotide
  source 1..20: mol_type = other DNA
aagggttcagtgcgtgggcg

SEQ ID NO: 177 - DNA, 26 bp, synthetic construct
  misc_feature 1..26: note = Description of Artificial Sequence: Syntheticoligonucleotide
  source 1..26: mol_type = other DNA
gatccgccacaacatcgaggacggca

SEQ ID NO: 178 - DNA, 4215 bp, synthetic construct
  misc_feature 1..4215: note = Description of Artificial Sequence: Syntheticpolynucleotide
  source 1..4215: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacggatcccccaagaagaagaggaaagtctcgagcgactacaaagaccatgacggtgattataaagatcatgacatcgattacaaggatgacgatgacaaggctgcaggatga

SEQ ID NO: 179 - DNA, 4260 bp, synthetic construct
  misc_feature 1..4260: note = Description of Artificial Sequence: Syntheticpolynucleotide
  source 1..4260: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacggatccggcggaggcggaagcgggaaaagaaccgccgacggcagcgaattcgagcccaagaagaagaggaaagtctcgagcggaggcgactacaaagaccatgacggtgattataaagatcatgacatcgattacaaggatgacgatgacaagtga

SEQ ID NO: 180 - DNA, 5562 bp, synthetic construct
  misc_feature 1..5562: note = Description of Artificial Sequence: Syntheticpolynucleotide
  source 1..5562: mol_type = other DNA
atgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 181 - DNA, 5412 bp, synthetic construct
  misc_feature 1..5412: note = Description of Artificial Sequence: Syntheticpolynucleotide
  source 1..5412: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 182 - DNA, 1536 bp, synthetic construct
  misc_feature 1..1536: note = Description of Artificial Sequence: Syntheticpolynucleotide
  source 1..1536: mol_type = other DNA
atgaaatgtctgctgtacctggctttcctgttcatcggcgtgaactgcaagttcaccatcgtgttccctcacaaccagaagggcaactggaaaaatgtgcctagcaactaccactactgtcctagctctagcgaccttaattggcataacgacctgatcggcacagccctgcaggtgaagatgcctaagagccacaaggccatccaggccgacggatggatgtgccacgccagcaagtgggtcacaacctgtgacttcagatggtacggccctaaatacattacccactctatcagaagcttcaccccttctgtggaacaatgtaaagagtccattgagcagacaaagcagggcacctggctgaaccccggcttccccccccagagctgcggctacgccaccgttaccgatgccgaggccgtgatcgtgcaggtgacacctcaccacgtgctggtcgatgagtacaccggcgaatgggtggacagccaatttatcaacggcaaatgcagcaattacatctgccccaccgtgcacaacagcaccacctggcacagcgattacaaggtgaaaggcctgtgcgacagcaacctgatctctatggacatcaccttcttcagcgaggacggcgagctgtctagtctgggcaaggaaggcacaggttttcggagcaactacttcgcctacgagactggcggcaaggcctgcaagatgcagtactgcaagcactggggcgttagactgccttcaggcgtgtggttcgagatggccgataaggacctgttcgccgctgccagattcccagagtgccctgagggcagctccatcagcgccccttcccagacctccgtggatgtgtccctgatccaggacgtggaaagaatcctggactacagcctctgtcaggagacatggtccaaaatcagagccggactccccattagccctgtggacctgagctacctggcccccaagaatcctggaaccggccccgccttcacaatcattaacggcaccctgaaatacttcgagaccagatacatccgggtggacatcgccgctcctatcctgtcaagaatggtgggcatgatttctggcacaacaacagagagggaactgtgggacgactgggccccttacgaggatgtggaaatcggcccaaacggcgtgctgcggaccagctcaggctataagttccccctgtacatgatcggccacggcatgctggattctgacctgcacctgagcagcaaggcccaggtctttgagcaccctcatatccaagacgccgccagccagctgcctgatgacgagagcctgttttttggagatacaggactgagcaagaaccccatcgagctggtggaaggctggtttagcagctggaagtccagcatagctagcttcttcttcatcatcggcctgatcatcggactgttcctggtgctgagagtggggatccacctgtgcatcaagctgaagcacaccaaaaagagacagatctacaccgacatcgagatgaaccggctggggaagtga

SEQ ID NO: 183 - DNA, 23 bp, synthetic construct
  misc_feature 1..23: note = Description of Artificial Sequence: Syntheticoligonucleotide
  source 1..23: mol_type = other DNA
tggggcccagactgagcacgtga

SEQ ID NO: 184 - DNA, 30 bp, synthetic construct
  misc_feature 1..30: note = Description of Artificial Sequence: Syntheticoligonucleotide
  source 1..30: mol_type = other DNA
tgtctgtgtgggtgagtgagtgtgtgcgtg

SEQ ID NO: 185 - DNA, 30 bp, synthetic construct
  misc_feature 1..30: note = Description of Artificial Sequence: Syntheticoligonucleotide
  source 1..30: mol_type = other DNA
caatccttggggcccagactgagcacgtga

SEQ ID NO: 186 - DNA, 20 bp, synthetic construct
  misc_feature 1..20: note = Description of Artificial Sequence: Syntheticoligonucleotide
  source 1..20: mol_type = other DNA
gaccccctccaccccgcctc

(19) United States
(12) Patent Application Publication
(10) Pub. No.: US 2026/0022349 A1
(43) Pub. Date: Jan. 22, 2026

(54) ENHANCED VIRUS-LIKE PARTICLES AND METHODS OF USE THEREOF FOR DELIVERY TO CELLS

(71) Applicants: The General Hospital Corporation, Boston, MA (US); President and Fellows of Harvard College, Cambridge, MA (US)

(72) Inventors: J. Keith Joung, Winchester, MA (US); Peter Cabeceiras, Boston, MA (US)

(21) Appl. No.: 19/194,667

(22) Filed: Apr. 30, 2025

Related U.S. Application Data
(63) Continuation of application No. 18/158,173, filed on Jan. 23, 2023, now Pat. No. 12,319,938.
Application No. 18/158,173 is a continuation of application No. PCT/US2021/043151, filed on Jul. 26, 2021.
(60) Provisional application No. 63/056,125, filed on Jul. 24, 2020.

(51) Int. Cl. C12N 7/00; C12N 9/22; C12N 15/11

(52) U.S. Cl. CPC C12N 7/00; C12N 9/22; C12N 15/11; C12N 2310/20; C12N 2760/20223; C12N 2760/20252; C12N 2800/80

(57) ABSTRACT

Enhanced virus-like particles (eVLPs), comprising a membrane comprising a phospholipid bilayer with one or more virally-derived glycoproteins on the external side; and a cargo disposed in the core of the eVLP on the inside of the membrane, wherein the eVLP does not comprise an exogenous gag/pol protein, and methods of use thereof for delivery of the cargo to cells.

### CLAIM OF PRIORITY

This application is a continuation of U.S. patent application Ser. No. 18/158,173, filed on Jan. 23, 2023, which is a continuation of International Patent Application No. PCT/US2021/043151, filed on Jul. 26, 2021, which claims the benefit of U.S. Provisional Patent Application Ser. No. 63/056,125, filed on Jul. 24, 2020. The entire contents of the foregoing are hereby incorporated by reference.

### FEDERALLY SPONSORED RESEARCH OR DEVELOPMENT

This invention was made with Government support under Grant No. GM118158 awarded by the National Institutes of Health. The Government has certain rights in the invention.

### SEQUENCE LISTING

This application contains a Sequence Listing that has been submitted electronically as an XML file named “40978-0358002_SL_ST26.XML.” The XML file, created on Apr. 30, 2025, is 268,384 bytes in size. The material in the XML file is hereby incorporated by reference in its entirety.

### TECHNICAL FIELD

Described herein are enhanced virus-like particles (eVLPs), comprising a membrane comprising a phospholipid bilayer with one or more virally-derived glycoproteins on the external side; and a cargo disposed in the core of the eVLP on the inside of the membrane, wherein the eVLP does not comprise a protein from any human endogenous or exogenous viral gag or pol, and methods of use thereof for delivery of the cargo to cells.

### BACKGROUND

Delivery of cargo such as proteins, nucleic acids, and/or chemicals into the cytosol of living cells has been a significant hurdle in the development of biological therapeutics.

### SUMMARY

Described herein are enhanced virus-like particles (eVLPs) that are capable of packaging and delivering a wide variety of payloads, e.g., biomolecules including nucleic acids (DNA, RNA) or proteins, chemical compounds including small molecules, and/or other molecules, and any combination thereof, into eukaryotic cells. The non-viral eVLP systems described herein have the potential to be simpler, more efficient and safer than conventional, artificially-derived lipid/gold nanoparticles and viral particle-based delivery systems, at least because eVLPs have no virus-derived components except for ENV, eVLPs can utilize but do not require chemical-based dimerizers, and eVLPs have the ability to package and deliver specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, internal ribosomal entry site containing RNA, etc.), proteins, chemical compounds and/or molecules, and combinations of the above listed cargos (e.g. AAV particles). The eVLPs described herein are different from conventional retroviral particles, virus-like particles (VLPs), exosomes and other previously described extracellular vesicles that can be loaded with cargo because of the membrane configuration, vast diversity of possible cargos that are enabled by novel, innovative loading strategies, the lack of a limiting DNA/RNA length constraint, the lack of proteins derived from any viral gag or pol, and the mechanism of cellular entry.

Described herein are compositions and methods for cargo delivery that can be used with a diverse array of protein and nucleic acid molecules, including genome editing, epigenome modulation, transcriptome editing and proteome modulation reagents, that are applicable to many disease therapies.

Thus, provided herein are eVLPs that include a membrane comprising a phospholipid bilayer with one or more virally-derived glycoproteins (e.g., as shown in Table 1) on the external side; and optionally a cargo disposed in the core of the eVLP on the inside of the membrane, wherein the eVLP does not comprise any gag and/or pol protein.

Also provided herein are methods for delivering a cargo to a target cell, e.g., a cell in vivo or in vitro. The methods include contacting the cell with an eVLP as described herein comprising the biomolecule and/or chemical as cargo.

Additionally provided herein are methods for producing an eVLP, e.g., comprising a biomolecular cargo. The methods include providing a cell expressing one or more virally-derived glycoproteins (ENV) (e.g., as shown in Table 1), and a cargo biomolecule and/or chemical, wherein the cell does not express an exogenous gag and/or pol protein; and maintaining the cell under conditions such that the cells produce eVLPs.

In some embodiments, the methods include harvesting and optionally purifying and/or concentrating the produced eVLPs.

In some embodiments, the methods include using cells that have or have not been manipulated to express any exogenous proteins except for an ENV (e.g., as shown in Table 1), and, if desired, a plasma membrane recruitment domain (e.g., as shown in Table 6). In this embodiment, the “empty” particles that are produced can be loaded with cargo by utilizing nucleofection, lipid, polymer, or CaCl₂ transfection, sonication, freeze thaw, and/or heat shock of purified particles mixed with cargo. In all embodiments, producer cells do not express any viral gag protein. This type of loading allows for cargo to be unmodified by fusions to plasma membrane recruitment domains and represents a significant advancement from previous VLP technology.

Also provided herein are cells expressing one or more virally-derived glycoproteins (e.g., as shown in Table 1), and a cargo, wherein the cell does not express an exogenous gag protein. In some embodiments, the cells are primary or stable human cell lines, e.g., Human Embryonic Kidney (HEK) 293 cells or HEK293 T cells. In some embodiments, the outer surface of the particle could contain scFvs, nanobodies, darpins, and/or other targeting peptides to enable cell-specific entry.

In some embodiments, the biomolecule cargo is a therapeutic or diagnostic protein or nucleic acid encoding a therapeutic or diagnostic protein.

In some embodiments, the cargo is a chemical compound or molecule.

In some embodiments, the chemical molecule is a trigger for protein-protein dimerization of multimerization, such as the A/C heterodimerizer or rapamycin.

In some embodiments, the chemical compound is a DNA PK inhibitor, such as M3814, NU7026, or NU7441 which potently enhance homology directed repair gene editing.

In some embodiments, the cargo is a gene editing reagent.

In some embodiments, the gene editing reagent comprises a zinc finger (ZF), transcription activator-like effector (TALE), and/or CRISPR-based genome editing or modulating protein; a nucleic acid encoding a zinc finger (ZF), transcription activator-like effector (TALE), and/or CRISPR-based genome editing or modulating protein; or a ribonucleoprotein complex (RNP) comprising a CRISPR-based genome editing or modulating protein.

In some embodiments, the gene editing reagent is selected from the proteins listed in Tables 2, 3, 4 & 5.

In some embodiments, the gene editing reagent comprises a CRISPR-based genome editing or modulating protein, and the eVLP further comprises one or more guide RNAs that bind to and direct the CRISPR-based genome editing or modulating protein to a target sequence.

In some embodiments, the cargo comprises a covalent or non-covalent connection to a plasma membrane recruitment domain, preferably as shown in Table 6. Covalent connections, for example, can include direct protein-protein fusions generated from a single reading frame, inteins that can form peptide bonds, other proteins that can form covalent connections at R-groups and/or RNA splicing. Non-covalent connections, for example, can include DNA/DNA, DNA/RNA, and/or RNA/RNA hybrids (nucleic acids base pairing to other nucleic acids via hydrogen-bonding interactions), protein domains that dimerize or multimerize with or without the need for a chemical compound/molecule to induce the protein-protein binding, single chain variable fragments, nanobodies, affibodies, proteins that bind to DNA and/or RNA, proteins with quaternary structural interactions, optogenetic protein domains that can dimerize or multimerize in the presence of certain light wavelengths, and/or naturally reconstituting split proteins.

In some embodiments, the cargo comprises a fusion to a dimerization domain or protein-protein binding domain that may or may not require a molecule to trigger dimerization or protein-protein binding.

In some embodiments, the producer cells are FDA-approved cells lines, allogenic cells, and/or autologous cells derived from a donor.

In some embodiments, the full or active peptide domains of human CD47 may be incorporated in the eVLP surface to reduce immunogenicity.

Examples of AAV proteins included here are AAV REP 52, REP 78, and VP1-3. The capsid site where proteins can be inserted is T138 starting from the VP1 amino acid counting. Dimerization domains could be inserted at this point in the capsid, for instance.

Examples of dimerization domains included here that may or may not need a small molecule inducer are dDZF1, dDZF2, DmrA, DmrB, DmrC, FKBP, FRB, GCN4 scFv, 10×/24× GCN4, GFP nanobody and GFP.

Examples of split inteins included here are Npu DnaE, Cfa, Vma, and Ssp DnaE.

Examples of other split proteins included here that make a covalent bond together are Spy Tag and Spy Catcher.

Examples of RNA binding proteins included here are MS2, Com, and PP7.

Examples of synthetic DNA-binding zinc fingers included here are ZF6/10, ZF8/7, ZF9, MK10, Zinc Finger 268, and Zinc Finger 268/NRE.

Examples of proteins that multimerize as a result of quaternary structure included here are E. coli ferritin, and the other chimeric forms of ferritin.

Examples of optogenetic “light-inducible proteins” included here are Cry2, CIBN, and Lov2-Ja.

Examples of peptides the enhance transduction included here are L17E, Vectofusin, KALA, and the various forms of nisin.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1: Depiction of exemplary T2eVLP/T4eVLP production and transduction for RNP/protein delivery. All eVLP expression constructs are stably integrated in the genome of the producer cell. Construct 1-1 corresponds to the phospholipid bilayer recruitment domain. 1-2 corresponds to the cargo. 2 corresponds to an optional guide RNA. 1-1 and 1-2 is translated in the cytosol where it complexes with guide RNA before it is recruited to the phospholipid bilayer. 3 corresponds to a virally-derived glycoprotein (VSVG). The virally-derived glycoprotein is expressed as a transmembrane protein on the plasma membrane and helps to drive budding of cargo-containing eVLPs from the plasma membrane to extracellular space. These particles are purified and are able to fuse with target cells and deliver cargo by interacting with surface receptors at the target cell surface.

FIG. 2: Depiction of purified eVLPs entering a target cell and delivering cargo to the cytosol. Importantly, the phospholipid bilayer recruitment domain allows cargo to enter the target cell nucleus as long as cargo possesses a nuclear localization sequence.

FIG. 3: Cas9 RNP was delivered in VSVG-pseudotyped T2eVLPs with or without a PH domain from hPLC61 or hAKT1. The PH domains were fused to the N-terminus of Cas9 via a 10 amino acid glycine/serine polypeptide linker. HepG2, U2OS, HEK293T, CFPAC1, BeWo, Jurkat, K562, and primary T cells were treated with purified and 100× concentrated T2eVLPs for 72 hours. Percent targeted gene modification of VEGF site #3 was determined by amplicon sequencing. The x-axis labels correspond to the contents of each T2eVLP preparation. Cas9 (no fusion) lacked a PH domain fusion. Benzonase (Benz) was used to degrade RNA and DNA outside of VLPs, and a Benzonase treated sample was included as a control.

FIG. 4: Depiction of T1eVLP/T3eVLP production. Plasmid DNA constructs involved in the transfection encode cargo, an optional guide RNA and a virally-derived glycoprotein (VSVG). Plasmids, or other types of DNA molecules, will be distributed throughout the production cell, so constructs located in the nucleus will express eVLP components and cargo, and constructs located near the plasma membrane or endosomes will be encapsulated within budding eVLPs.

FIGS. 5A-B: Exemplary T1eVLP-delivered spCas9 genome editing in vitro. A) U2OS eGFP and HEK293 eGFP cell lines transduced with VLPs containing Rous sarcoma virus gag fused to spCas9 and sgRNA, T1eVLPs containing PLC pleckstrin homology (PH) fused to spCas9 and sgRNA, or VLPs containing Rous sarcoma virus gag fused to the SV40 nuclear localization sequence (NLS) and sgRNA. The sgRNA targets GFP. Flow cytometry or T7E1 is performed 72 hours after transduction. The Rous sarcoma virus gag VLPs serve as controls. B) T7E1 analysis of a subpopulation of U2OS or 293 cells from the experiment in FIG. 5A. eVLPs and VLPs are pseudotyped with VSVG.

FIG. 6: Exemplary T1eVLP-delivered spCas9 genome editing in vitro. U2OS cells transduced with T1eVLPs containing PLC PH fused to spCas9 targeted to HEK site #3 or VEGF site #2. eVLPs are pseudotyped with VSVG. Gene modification is measured by amplicon sequencing.

FIG. 7: Exemplary T1eVLP-delivered spCas9 genome editing in vitro. U2OS cells transduced with T1eVLPs containing PLC PH or hAkt PH fused to spCas9 targeted to VEGF site #3. eVLPs are pseudotyped with VSVG. Gene modification is measured by amplicon sequencing.

FIG. 8: Exemplary TleVLP-delivered spCas9 base editing in vitro. HEK293T cells transduced with VLPs containing Rous sarcoma virus gag fused to spCas9 BE3 or Gam-BE4 with sgRNA targeted to VEGF site #2, or TieVLPs containing PLC PH fused to spCas9 BE3 or Gam-BE4 with sgRNA targeted to VEGF site #2. eVLPs and VLPs are pseudotyped with VSVG. Gene modification is measured by amplicon sequencing. The Rous sarcoma virus gag VLP serves as a positive control. FIG. 8 discloses SEQ ID NO: 186.

FIG. 9: Exemplary T1eVLP-delivered spCas9 base editing in vitro. HEK293T cells transduced with T1eVLPs containing PLC PH fused to codon optimized spCas9 BE4 targeted to HEK site #3. eVLPs are pseudotyped with VSVG. Gene modification is measured by amplicon sequencing. FIG. 9 discloses SEQ ID NO: 183.

FIG. 10: Exemplary T1eVLP-delivered spCas9 base editing in vitro. HEK293T cells transduced with T1eVLPs containing PLC PH fused to codon optimized spCas9 ABE targeted to VEGF site #3. eVLPs are pseudotyped with VSVG. Gene modification is measured by amplicon sequencing. FIG. 10 discloses SEQ ID NOS 184, 184 and 184, respectively, in order of appearance.

FIG. 11: Exemplary T1eVLP-delivered spCas9 base editing in vitro. HEK293T cells transduced with T1eVLPs containing PLC PH fused to codon optimized spCas9 ABE targeted to HEK site #3. eVLPs are pseudotyped with VSVG. Gene modification is measured by amplicon sequencing. FIG. 11 discloses SEQ ID NOS 185, 185 and 185, respectively, in order of appearance.

FIG. 12: Exemplary T1eVLP-delivered asCas12a genome editing in vitro. HEK293 cells transduced with VLPs containing Rous sarcoma virus gag or T1eVLPs containing PLC PH fused to asCas12a. VLPs and eVLPs are targeted to FANCF site #1 by crRNA. Gene modification is measured by T7E1. The Rous sarcoma virus gag VLP serves as a positive control.

FIG. 13: Exemplary T1eVLP-delivered spCas9 genome editing in vitro. HEK293 cells transduced with T1eVLPs containing PLC PH fused to RNA binding protein MS2. MS2 binds to MS2 stem loops in the sgRNA, which is complexed with Cas9, and MS2 is fused to a PH domain for efficient eVLP loading. eVLPs are targeted to GFP site #1 by sgRNA. Gene modification is measured by T7E1.

FIG. 14: Exemplary T1eVLP-delivered spCas9 genome editing in vitro. HEK293 cells transduced with T1eVLPs containing PLC PH fused to dimerization domain (DmrC). In the presence of A/C Heterodimerizer molecule, DmrC binds to DmrA which is directly fused to Cas9. eVLPs are targeted to GFP site #1 by sgRNA. Gene modification is measured by T7E1.

FIG. 15: Exemplary T1eVLP-delivered asCas9 genome editing in vitro. HEK293 cells transduced with T1eVLPs containing PLC PH fused to GNC4 protein domain repeats. An scFv binds to the GCN4 repeats, and scFvs are directly fused to Cas9. eVLPs are targeted to GFP site #1 by sgRNA. Gene modification is measured by T7E1.

FIGS. 16-40: Schematic illustrations of various exemplary eVLP configurations and possible cargo.

### DETAILED DESCRIPTION

Therapeutic proteins and nucleic acids hold great promise, but for many of these large biomolecules delivery into cells is a hurdle to clinical development. Genome editing reagents such as zinc finger nucleases (ZFNs) or RNA-guided, enzymatically active/inactive DNA binding proteins such as Cas9 have undergone rapid advancements in terms of specificity and the types of edits that can be executed, but the hurdle of safe in vivo delivery still precludes efficacious gene editing therapies. Described herein are various embodiments of enhanced virus-like particles (eVLPs), as well as characteristics of various embodiments of eVLPs that provide a novel and optimal platform for the delivery of genome editing reagents, and contrasts eVLPs with canonical delivery modalities.

Retroviral particles, such as lentivirus, have been developed to deliver RNA that is reverse transcribed to DNA that may or may not be integrated into genomic DNA. VLPs have been developed that mimic virus particles in their ability to self-assemble, but are not infectious as they lack some of the core viral genes. Both lentiviral and VLP vectors are typically produced by transiently transfecting a producer cell line with plasmids that encode all components necessary to produce lentiviral particles or VLP. One major flaw that we have discovered regarding lentiviral particles and VSVG-based VLPs that are produced by this conventional transient transfection method is that, in addition to their conventional cargo, these particles package and deliver plasmid DNA that was used in the initial transient transfection. This unintended plasmid DNA delivery can be immunogenic and cause undesirable effects, such as plasmid DNA being integrated into genomic DNA. It is important to specify the type of biomolecules/chemicals that are to be delivered within particles, and eVLPs have been designed to possess this germane capability.

The eVLPs described herein can deliver a wide variety of cargo including DNA only, DNA+RNA+protein, or RNA+protein. Importantly, eVLPs are the first VSVG-based VLP delivery modality that can control the form of the cargo (DNA, protein, and/or RNA). Previously described VSVG-based vesicles and viral particles package and deliver unwanted plasmid DNA (or other types of DNA-based gene expression constructs) introduced into particle producer cells via transient transfection in addition to the intended protein and/or RNA cargo(s).

Another aspect of eVLPs is the ENV protein on the surface of the eVLP. Without wishing to be bound by theory, the ENV protein alone is responsible for eVLP particle generation and the ability of eVLPs to efficiently deliver cargo into cells. Lentivirus and VLPs commonly require GAG and ENV proteins to drive particle formation via budding off of the plasma membrane of producer cells into the cell culture medium. In addition, the majority of retroviral ENV proteins require post-translational modifications in the form of proteolytic cleavage of the intracellular domain (ICD) of the ENV protein in order to activate the fusogenicity of the ENV protein; this is essential for viral infectivity. The envelope proteins described in Table 1 are all derived from viruses. However, these eVLP ENV proteins do not require exogenous GAG for particle formation and they do not require ICD cleavage for fusogenicity.¹⁻³ The ENV is the only virally-derived component of eVLPs, and these ENV glycoproteins on the external surface of the eVLPs are used to facilitate fusion/entry of eVLPs into the target cell because they are known to be naturally fusogenic. In addition, eVLPs are different from previously described viral particles, VLPs, and extracellular vesicles because eVLPs are composed of a mixture of ectosomes and exosomes which can be separated by purification, if desired. Because of the above mentioned design simplifications and optimizations, eVLPs are particularly suited for delivery of cargo including DNA, RNA, protein, or combinations of biomolecules and/or chemicals, such as DNA-encoded or RNP-based genome editing reagents.

Large biomolecules including proteins and protein complexes such as genome editing reagents, especially CRISPR-CAS, zinc finger, and TAL-nuclease-based reagents, have the potential to become in vivo therapeutics for the treatment of a number of diseases including genetic diseases, but techniques for delivering these reagents into cells are severely limiting or unsafe for patients. Conventional therapeutic monoclonal antibody delivery is successful at utilizing direct injection for proteins. Unfortunately, strategies for direct injection of gene editing proteins, such as Cas9, are hampered by immunogenicity, degradation, ineffective cell specificity, and inability to cross the plasma membrane or escape endosomes/lysosomes.⁴⁻¹⁰ More broad applications of protein therapy and gene editing could be achieved by delivering therapeutic protein cargo to the inside of cells. Cas9, for example, cannot efficiently cross the phospholipid bilayer to enter into cells, and has been shown to have innate and adaptive immunogenic potential.⁴⁻⁸ Therefore, it is not practical or favorable to deliver Cas9 by direct injection or as an external/internal conjugate to lipid, protein or metal-based nanoparticles that have cytotoxic and immunogenic properties and often yield low levels of desired gene modifications.⁹⁻²⁰ Nanoparticles that encapsulate cargo are another delivery strategy that can be used to deliver DNA, protein, RNA and RNPs into cells⁹⁻¹⁸ Nanoparticles can be engineered for cell specificity and can trigger endocytosis and subsequent endosome lysis. However, nanoparticles can have varying levels of immunogenicity due to an artificially-derived vehicle shell.⁹⁻²⁰ Many nanoparticles rely on strong opposing charge distributions to maintain particle structural integrity, and the electrostatics can make it toxic and unfit for many in vivo therapeutic scenarios.⁹ Nanoparticles that deliver RNA have had successes in recent clinical trials, but most have only been used to deliver siRNA or shRNA. Toxicity from such nanoparticles is still a major concern.⁹ Nanoparticles that deliver mRNA coding for genome editing RNPs have also been a recent success, but these create a higher number of off-target effects compared to protein delivery and RNA stability is lower than that of protein.¹⁷ Nanoparticles that deliver genome editing RNPs and DNA have been a significant breakthrough because they can leverage both homology directed repair (HDR) and non-homologous end joining (NHEJ), but exhibit prohibitively low gene modification frequencies in vitro and in vivo, and therefore currently have limited applications in vivo as a gene editing therapeutic.¹⁵

Currently, the clinical standard vehicles for delivering genome editing therapeutics are adeno-associated virus (AAV). Although AAV vectors are a promising delivery modality that can successfully deliver DNA into eukaryotic cells, AAV cannot efficiently package and deliver DNA constructs larger than 4.5 kb and this precludes delivery of many CRISPR-based gene editing reagents that require larger DNA expression constructs. CRISPR-based gene editing reagents can be split into multiple different AAV particles, but this strategy drastically reduces delivery and editing efficiency. Depending on the dose required, AAV and adenoviral vectors can have varying levels of immunogenicity. In addition, inverted-terminal repeats (ITRs) in the AAV DNA construct can promote the formation of spontaneous episomes leading to prolonged expression of genome editing reagents and increased off-target effects. ITRs can also promote the undesired integration of AAV DNA into genomic DNA.²¹⁻²⁴ Recently, VLPs have been utilized to deliver mRNA and protein cargo into the cytosol of cells.^{2,3,25-30} VLPs have emerged as a substitute delivery modality for retroviral particles. VLPs can be designed to lack the ability to integrate retroviral DNA, and to package and deliver protein/RNP/DNA. However, most VLPs, including recently conceived VLPs that deliver genome editing reagents known to date, utilize HIV or other virally-derived gag-pol protein fusions and viral proteases to generate retroviral-like particles.^{25-27,29,30} Secondly, some VLPs containing RGNs also must package and express guide RNAs from a lentiviral DNA transcript.²⁷ Thirdly, some VLPs require a viral protease in order to form functional particles and release genome editing cargo.^{25-27,29} Since this viral protease recognizes and cleaves at multiple amino acid motifs, it can cause damage to the protein cargo which could be hazardous for therapeutic applications. Fourthly, most published VLP modalities that deliver genome editing proteins to date exhibit low in vitro and in vivo gene modification efficiencies due to low packaging and transduction efficiency.²⁵⁻²⁷ Fifthly, the complex viral genomes utilized for these VLP components possess multiple reading frames and employ RNA splicing that could result in spurious fusion protein products being delivered.^{25-27,29,30} Sixthly, the presence of reverse transcriptase, integrase, capsid and a virally-derived envelope protein in these VLPs is not ideal for most therapeutic applications because of immunogenicity and off target editing concerns. Lastly, most retroviral particles, such as lentiviral particles, are pseudotyped with VSVG and nearly all described VLPs that deliver genome editing reagents hitherto possess and rely upon VSVG.^{2,3,25-30} We have discovered that VSVG-based particles that are formed by transiently transfecting producer cells package and deliver DNA that was transfected. The current versions of VSVG-based VLPs cannot prevent this inadvertent delivery of DNA and this impedes the use of VLPs in scenarios that necessitate minimal immunogenicity and off target effects.

Extracellular vesicles are another delivery modality that can package and deliver cargo within exosomes and ectosomes.^{31,32} Similar to VLPs, extracellular vesicles are comprised of a phospholipid bilayer from a mammalian cell. Unlike VLPs, extracellular vesicles lack viral components and therefore have limited immunogenicity. Whereas VLPs have a great ability to enter cells due to external fusogenic glycoproteins (VSVG) extracellular vesicles mainly rely on cellular uptake via micropinocytosis and this limits the delivery efficiency of extracellular vesicles.

eVLPs are a safer and more effective alternative than previously described VLPs, extracellular vesicles, AAVs and nanoparticles-especially for delivery of genome editing reagents-because eVLPs are composed of all human components except for a virally-derived glycoprotein that has been demonstrated to be safe in humans in a clinical trial of a HIV-1 gag vaccine (VSVG),³³ eVLPs lack all other retroviral components besides a safe glycoprotein, eVLPs have the ability to deliver DNA+RNP, or RNP alone while other previously described VLPs cannot prevent transient transfection DNA from being unintentionally packaged and delivered, eVLPs can deliver specialty DNA molecules while previously described VLPs, nanoparticles and AAVs cannot or do not, and eVLPs can be produced with cells that have been derived from patients (autologous eVLPs) and other FDA-approved cell lines (allogenic eVLPs) to further reduce the risks of adverse immune reactions. Here, we describe methods and compositions for producing, purifying, and administering eVLPs for in vitro and in vivo applications, e.g., of genome editing, epigenome modulation, transcriptome editing and proteome modulation. The desired editing outcome depends on the therapeutic context and will require different gene editing reagents. Streptococcus pyogenes Cas9 (spCas9) and acidaminococcus sp. Cas12a (functionalize) are two of the most popular RNA-guided enzymes for editing that leverages NHEJ for introducing stop codons or deletions, or HDR for causing insertions.³⁴⁻³⁶ Cas9-deaminase fusions, also known as base editors, are the current standard for precise editing of a single nucleotide without double stranded DNA cleavage.^{37,38} Importantly, these methods address the phenomenon of inadvertent DNA delivery in VLPs and the first to control for the type of biomolecule to be delivered (DNA, RNA, and/or protein) thereby increasing the types of therapeutic in vivo genome modifications that are possible and minimizing deleterious off target effects.

### Section 1: EVLP-Mediated Delivery of DNAs, Proteins and RNAs

Conventional VLPs that have been engineered to encapsulate and deliver protein-based cargo commonly fuse cargo to the INT or GAG polyprotein.^{25-27,29,30,39,40} After transient transfection of production plasmid DNA constructs, these protein fusions are translated in the cytosol of conventional VLP production cell lines, the gag matrix is acetylated and recruited to the cell membrane, and the gag fusions are encapsulated (transient transfection DNA is also unintentionally encapsulated) within VLPs as VLPs bud off of the membrane into extracellular space.

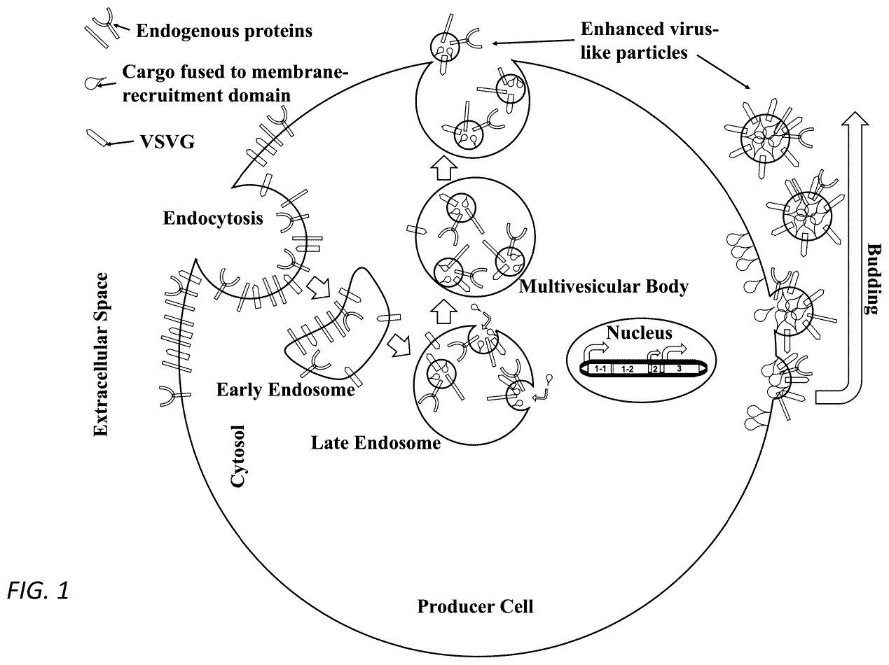

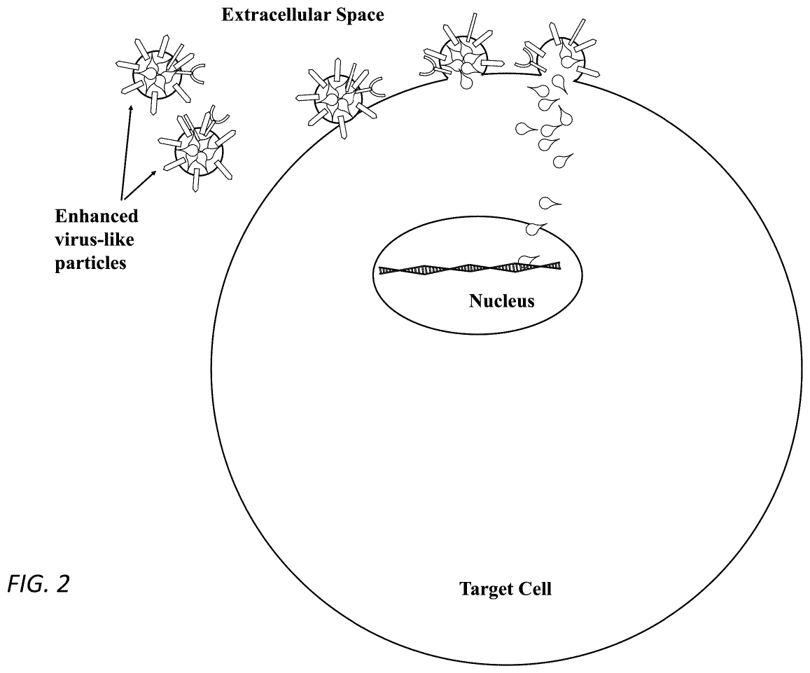

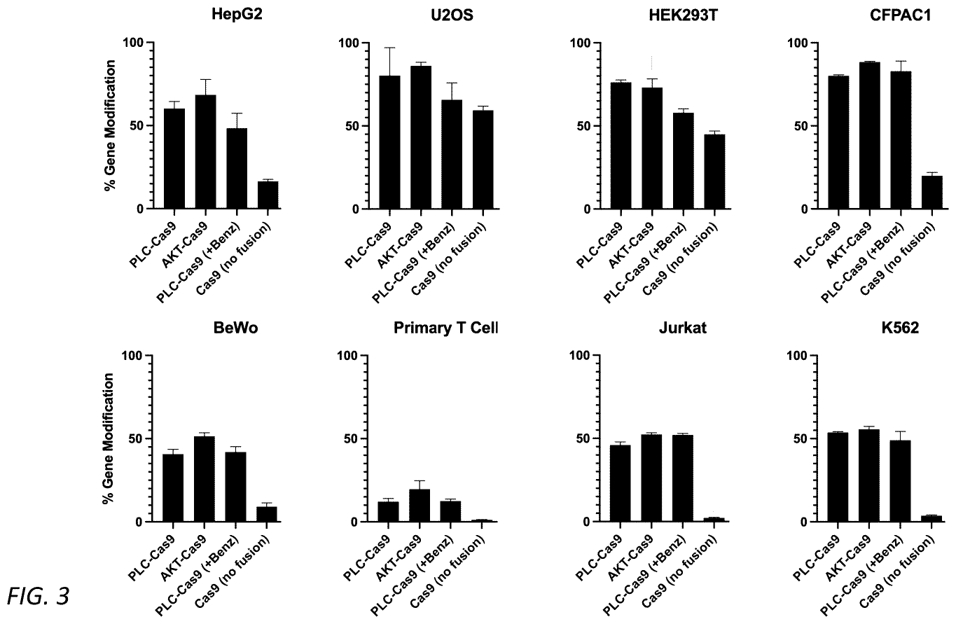

In contrast, in some embodiments the eVLPs described herein can package protein-based cargo by integrating all production DNA into the genomic DNA of production cell lines. Once cell lines are created, protein delivery eVLPs can be produced in a constitutive or inducible fashion. Proteins are packaged into eVLP by fusing select human-derived phospholipid bilayer recruitment domains to protein-based cargo (e.g., as shown in Table 6). One such human-derived phospholipid bilayer recruitment domain used for this purpose is a human pleckstrin homology (PH) domain. PH domains interact with phosphatidylinositol lipids and proteins within biological membranes, such as PIP2, PIP3, βγ-subunits of GPCRs, and PKC.^{41,42} Alternatively, the human Arc protein can be fused to protein-based cargo to recruit cargo to the cytosolic side of the phospholipid bilayer.⁴³ These human-derived phospholipid bilayer recruitment domains can be fused to the N-terminus or C-terminus of protein-based cargo via polypeptide linkers of variable length regardless of the location or locations of one or more nuclear localization sequence(s) (NLS) within the cargo. Preferably, the linker between protein-based cargo and the phospholipid bilayer recruitment domain is a polypeptide linker 5-20, e.g., 8-12, e.g., 10, amino acids in length primarily composed of glycines and serines. The human-derived phospholipid bilayer recruitment domain localizes the cargo to the phospholipid bilayer and this protein cargo is packaged within eVLPs that utilize a glycoprotein to trigger budding off of particles from the producer cell into extracellular space (FIG. 1). These human-derived domains and proteins can facilitate for localization of cargo to the cytosolic face of the plasma membrane within the eVLP production cells, and they also allow for cargo to localize to the nucleus of eVLP-transduced cells without the utilization of exogenous retroviral gag/pol or chemical and/or light-based dimerization systems (FIG. 2). The delivery of Cas9, for example, is significantly more efficient with a fusion to a plasma membrane recruitment domain compared to without a plasma membrane recruitment domain (FIG. 3).

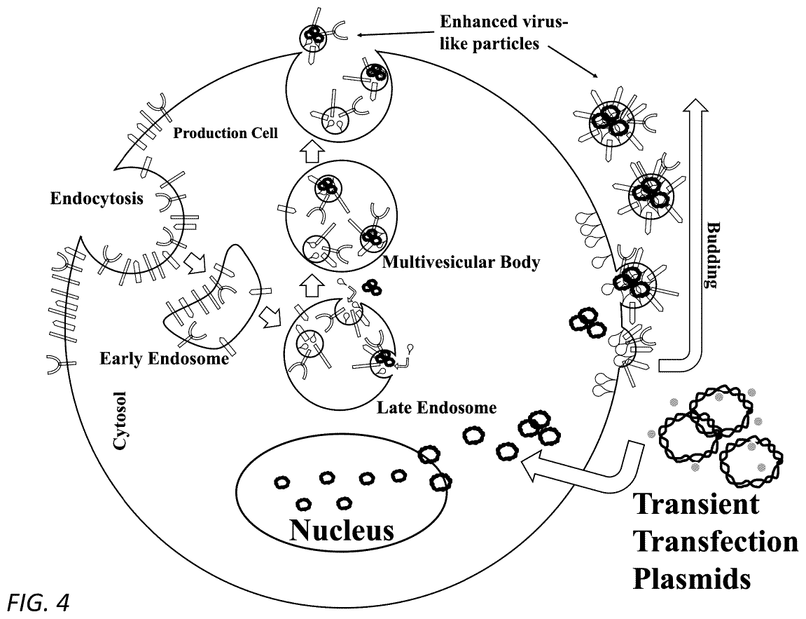

In some embodiments, eVLPs can also package and deliver a combination of DNA and RNA if eVLPs are produced via transient transfection of a production cell line. DNA that is transfected into cells will possess size-dependent mobility such that a fraction of the transfected DNA will remain in the cytosol while another fraction of the transfected DNA will localize to the nucleus.⁴⁴⁻⁴⁶ One fraction of the transfected DNA in the nucleus will expressed components needed to create eVLPs and the other fraction in the cytosol/near the plasma membrane will be encapsulated and delivered in eVLPs (FIG. 4).

eVLP “Cargo” refers to a any payload that can be delivered, including chemicals, e.g., small molecule compounds, and biomolecules, including DNA, RNA, RNP, proteins, and combinations thereof, including combinations of DNA and RNP, RNP, combinations of DNA and proteins, or proteins, as well as viruses and portions thereof, e.g., for therapeutic or diagnostic use, or for the applications of genome editing, epigenome modulation, and/or transcriptome modulation. In order to simplify these distinctions, a combination of DNA and RNP will be referred to herein as type 1 cargo (T1eVLPs), RNP will be referred to herein to as type 2 cargo (T2eVLPs), a combination of DNA and proteins will be referred to herein to as type 3 cargo (T3eVLPs), and proteins will be referred to herein to as type 4 cargo (T4eVLPs). RNA in this context includes, for example, single guide RNA (sgRNA), Clustered Regularly Interspaced Palindromic Repeat (CRISPR) RNA (crRNA), and/or mRNA coding for cargo.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The cargo is limited by the diameter of the particles, e.g., which in some embodiments range from 30 nm to 500 nm.

Cargo developed for applications of genome editing also includes nucleases and base editors. Nucleases include FokI and AcuI ZFNs and Transcription activator-like effector nucleases (TALENs) and CRISPR based nucleases or a functional derivative thereof (e.g., as shown in Table 2) (ZFNs are described, for example, in United States Patent Publications 20030232410; 20050208489; 20050026157; 20050064474; 20060188987; 20060063231; and International Publication WO 07/014275) (TALENs are described, for example, in United States Patent Publication U.S. Pat. No. 9,393,257B2; and International Publication WO2014134412A1) (CRISPR based nucleases are described, for example, in United States Patent Publications U.S. Pat. No. 8,697,359B1; US20180208976A1; and International Publications WO2014093661A2; WO2017184786A8).³⁴⁻³⁶ Base editors that are described by this work include any CRISPR based nuclease orthologs (wt, nickase, or catalytically inactive (CI)), e.g., as shown in Table 2, fused at the N-terminus to a deaminase or a functional derivative thereof (e.g., as shown in Table 3) with or without a fusion at the C-terminus to one or multiple uracil glycosylase inhibitors (UGIs) using polypeptide linkers of variable length (Base editors are described, for example, in United States Patent Publications US20150166982A1; US20180312825A1; U.S. Ser. No. 10/113,163B2; and International Publications WO2015089406A1; WO2018218188A2; WO2017070632A2; WO2018027078A8; WO2018165629A1).³⁷,38 In addition, prime editors are also compatible with eVLP delivery modalities (Prime editors are described, for example, in PMID: 31634902).

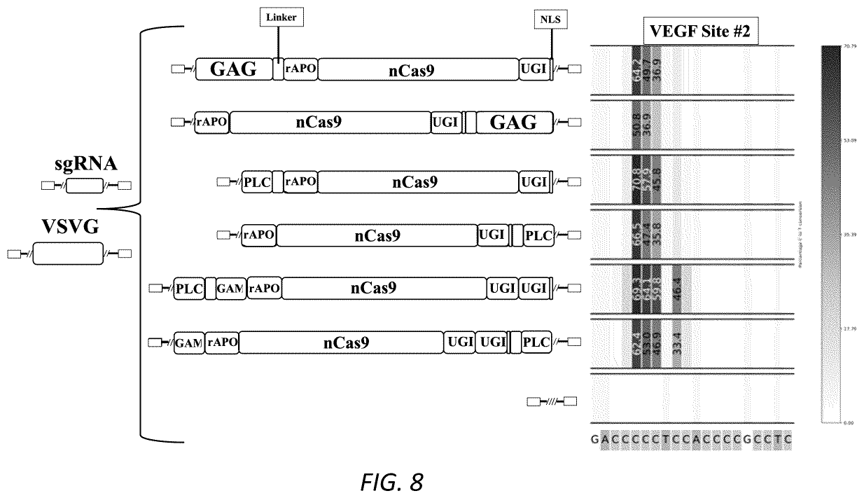

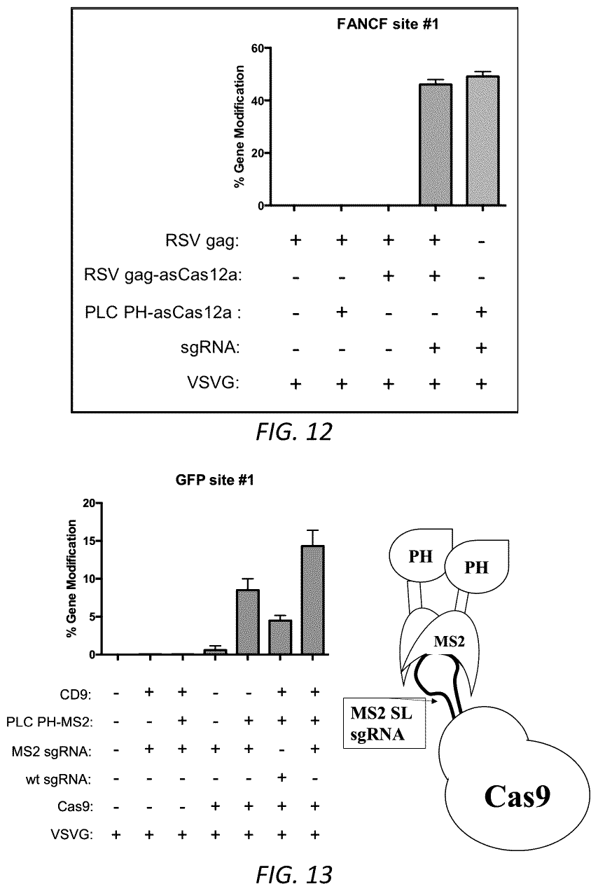

sgRNAs complex with genome editing reagents during the packaging process, and are co-delivered within eVLPs. To date, this concept has been validated in vitro by experiments that demonstrate the T1eVLP or T2eVLP delivery of RGN and CI RGN fused to deaminase and UGI (base editor) as protein for the purposes of site specific editing of exogenous and endogenous sites (FIGS. 3, 5, 6, 7, 8, 9, 10, 11 & 12). For example, T1eVLPs have been used to deliver Cas9 RNP to U2OS and HEK293 cells for the purposes of editing exogenous GFP, and endogenous HEK site #3and VEGF site #2 & #3 (FIGS. 4, 5, 6 & 7). In addition, T1eVLPs have been used to deliver BE3 and BE4 RNP to HEK293T cells for the purpose of base editing endogenous VEGF site #2 & #3 and HEK site #3 (FIGS. 8, 9, 10 & 11). T1eVLPs have also been used to deliver Cas12a RNP to HEK293 cells for the purposes of editing endogenous FANCF site #1 (FIG. 12).

Cargo designed for the purposes of epigenome modulation includes the CI CRISPR based nucleases, zinc fingers (ZFs) and TALEs fused to an epigenome modulator or combination of epigenome modulators or a functional derivative thereof connected together by one or more variable length polypeptide linkers (Tables 2 & 4). T1-T4 cargo designed for the purposes of transcriptome editing includes CRISPR based nucleases or any functional derivatives thereof in Table 5 or CI CRISPR based nucleases or any functional derivatives thereof in Table 5 fused to deaminases in Table 3 by one or more variable length polypeptide linkers.

The cargo can also include any therapeutically or diagnostically useful protein, DNA, RNP, or combination of DNA, protein and/or RNP. See, e.g., WO2014005219; U.S. Ser. No. 10/137,206; US20180339166; U.S. Pat. No. 5,892,020A; EP2134841B1; WO2007020965A1. For example, cargo encoding or composed of nuclease or base editor proteins or RNPs or derivatives thereof can be delivered to retinal cells for the purposes of correcting a splice site defect responsible for Leber Congenital Amaurosis type 10. In the mammalian inner ear, eVLP delivery of base editing reagents or HDR promoting cargo to sensory cells such as cochlear supporting cells and hair cells for the purposes of editing β-catenin (β-catenin Ser 33 edited to Tyr, Pro, or Cys) in order to better stabilize β-catenin could help reverse hearing loss.

In another application, eVLP delivery of RNA editing reagents or proteome perturbing reagents could cause a transitory reduction in cellular levels of one or more specific proteins of interest (potentially at a systemic level, in a specific organ or a specific subset of cells, such as a tumor), and this could create a therapeutically actionable window when secondary drug(s) could be administered (this secondary drug is more effective in the absence of the protein of interest or in the presence of lower levels of the protein of interest). For example, eVLP delivery of RNA editing reagents or proteome perturbing reagents could trigger targeted degradation of MAPK and PI3K/AKT proteins and related mRNAs in vemurafenib/dabrafenib-resistant BRAF-driven tumor cells, and this could open a window for the administration of vemurafenib/dabrafenib because BRAF inhibitor resistance is temporarily abolished (resistance mechanisms based in the MAPK/PI3K/AKT pathways are temporarily downregulated by eVLP cargo). This example is especially pertinent when combined with eVLPs that are antigen inducible and therefore specific for tumor cells.

In some embodiments, eVLPs could be used deliver factors, e.g., including the Yamanaka factors Oct3/4, Sox2, Klf4, and c-Myc, to cells such as human or mouse fibroblasts, in order to generate induced pluripotent stem cells.

In some embodiments, eVLPs could deliver dominant-negative forms of proteins in order to elicit a therapeutic effect.

eVLPs that are antigen-specific (i.e., tumor-antigen specific) could be targeted to cancer cells in order to deliver proapoptotic proteins BIM, BID, PUMA, NOXA, BAD, BIK, BAX, BAK and/or HRK in order to trigger apoptosis of cancer cells. Tumor antigens are known in the art and include

90% of pancreatic cancer patients present with unresectable disease. Around 30% of patients with unresectable pancreatic tumors will die from local disease progression, so it is desirable to treat locally advanced pancreatic tumors with ablative radiation, but the intestinal tract cannot tolerate high doses of radiation needed to cause tumor ablation. Selective radioprotection of the intestinal tract enables ablative radiation therapy of pancreatic tumors while minimizing damage done to the surrounding gastrointestinal tract. To this end, eVLPs could be loaded with dCas9 fused to the transcriptional repressor KRAB and guide RNA targeting EGLN. EGLN inhibition has been shown to significantly reduce gastrointestinal toxicity from ablative radiation treatments because it causes selective radioprotection of the gastrointestinal tract but not the pancreatic tumor.⁴⁷ Such fusion proteins, eVLPs, and methods of making and using the same are provided herein.

Unbound steroid receptors reside in the cytosol. After binding to ligands, these receptors will translocate to the nucleus and initiate transcription of response genes. eVLPs could deliver single chain variable fragment (scFv) antibodies to the cytosol of cells that bind to and disrupt cytosolic steroid receptors. For example, the scFv could bind to the glucocorticoid receptor and prevent it from binding dexamethasone, and this would prevent transcription of response genes, such as metallothionein JE which has been linked to tumorigenesis.⁴⁸

eVLPs can be indicated for treatments that involve targeted disruption of proteins. For example, eVLPs can be utilized for targeting and disrupting proteins in the cytosol of cells by delivering antibodies/scFvs to the cytosol of cells. Classically, delivery of antibodies through the plasma membrane to the cytosol of cells has been notoriously difficult and inefficient. This mode of protein inhibition is similar to how a targeted small molecule binds to and disrupts proteins in the cytosol and could be useful for the treatment of a diverse array of diseases.⁴⁹⁻⁵¹ Such fusion proteins, eVLPs, and methods of making and using the same are

In addition, the targeting of targeted small molecules is limited to proteins of a certain size that contain binding pockets which are relevant to catalytic function or protein-protein interactions. scFvs are not hampered by these limitations because scFvs can be generated that bind to many different moieties of a protein in order to disrupt catalysis and interactions with other proteins. For example, RAS oncoproteins are implicated across a multitude of cancer subtypes, and RAS is one of the most frequently observed oncogenes in cancer. For instance, the International Cancer Genome Consortium found KRAS to be mutated in 95% of their Pancreatic Adenocarcinoma samples. RAS isoforms are known to activate a variety of pathways that are dysregulated in human cancers, like the PI3K and MAPK pathways. Despite the aberrant roles RAS plays in cancer, no efficacious pharmacologic direct or indirect small molecule inhibitors of RAS have been developed and approved for clinical use. One strategy for targeting RAS could be eVLPs that can deliver specifically to cancer cells scFvs that bind to and disrupt the function of multiple RAS isoforms.⁴⁹⁻⁵¹

### Section 2: EVLP Composition, Production, Purification and Applications

eVLPs can be produced from producer cell lines that are either transiently transfected with at least one plasmid or stably expressing constructs that have been integrated into the producer cell line genomic DNA. In some embodiments, for T1 and T3eVLPs, if a single plasmid is used in the transfection, it should comprise sequences encoding one or more virally-derived glycoproteins (e.g., as shown in Table 1), cargo (e.g., a therapeutic protein or a gene editing reagent such as a zinc finger, transcription activator-like effector (TALE), and/or CRISPR-based genome editing/modulating protein and/or RNP such as those found in Tables 2, 3, 4 & 5), with or without fusion to a plasma membrane recruitment domain (e.g., as shown in Table 6), and a guide RNA, if necessary. Preferably, two to three plasmids are used in the transfection. These two to three plasmids can include the following (any two or more can be combined in a single plasmid):
1. A plasmid comprising sequences encoding a therapeutic protein or a genome editing reagent, with or without a fusion to a plasma membrane recruitment domain.
2. A plasmid comprising one or more virally-derived glycoproteins (e.g., as listed in Table 1).
3. If the genome editing reagent from plasmid 1 requires one or more guide RNAs, a plasmid comprising one or more guide RNAs apposite for the genome editing reagent in plasmid 1.

If it is desired to deliver a type of DNA molecule other than plasmid(s), the above-mentioned transfection can be performed with double-stranded closed-end linear DNA, episome, mini circle, double-stranded oligonucleotide and/or other specialty DNA molecules. Alternatively, for T2 and T4eVLPs, the producer cell line can be made to stably express the constructs (1 through 3) described in the transfection above.

As stated earlier, in some embodiments, the methods include using cells that have or have not been manipulated to express any exogenous proteins except for a viral envelope (e.g., as shown in Table 1), and, if desired, a plasma membrane recruitment domain (e.g., as shown in Table 6). In this embodiment, the “empty” particles that are produced can be loaded with cargo by utilizing nucleofection, lipid, polymer, or CaCl₂ transfection, sonication, freeze thaw, and/or heat shock of purified particles mixed with cargo. In all embodiments, producer cells do not express any gag protein. This type of loading allows for cargo to be unmodified by fusions to plasma membrane recruitment domains and represents a significant advancement from previous VLP technology.

The plasmids, or other types of specialty DNA molecules known in the art or described above, can also preferably include other elements to drive expression or translation of the encoded sequences, e.g., a promoter sequence; an enhancer sequence, e.g., 5′ untranslated region (UTR) or a 3′ UTR; a polyadenylation site; an insulator sequence; or another sequence that increases or controls expression (e.g., an inducible promoter element).

Preferably, appropriate producer cell lines are primary or stable human cell lines refractory to the effects of transfection reagents and fusogenic effects due to virally-derived glycoproteins. Examples of appropriate cell lines include Human Embryonic Kidney (HEK) 293 cells, HEK293 T/17 SF cells kidney-derived Phoenix-AMPHO cells, and placenta-derived BeWo cells. For example, such cells could be selected for their ability to grow as adherent cells, or suspension cells. In some embodiments, the producer cells can be cultured in classical DMEM under serum conditions, serum-free conditions, or exosome-free serum conditions. eVLPs, e.g., T1 and T3eVLPs, can be produced from cells that have been derived from patients (autologous eVLPs) and other FDA-approved cell lines (allogenic eVLPs) as long as these cells can be transfected with DNA constructs that encode the aforementioned eVLP production components by various techniques known in the art.

In addition, if it is desirable, more than one genome editing reagent can be included in the transfection. The DNA constructs can be designed to overexpress proteins in the producer cell lines. The plasmid backbones, for example, used in the transfection can be familiar to those skilled in the art, such as the pCDNA3 backbone that employs the CMV promoter for RNA polymerase II transcripts or the U6 promoter for RNA polymerase III transcripts. Various techniques known in the art may be employed for introducing nucleic acid molecules into producer cells. Such techniques include chemical-facilitated transfection using compounds such as calcium phosphate, cationic lipids, cationic polymers, liposome-mediated transfection, such as cationic liposome like LIPOFECTAMINE (LIPOFECTAMINE 2000 or 3000 and TransIT-X2), polyethyleneimine, non-chemical methods such as electroporation, particle bombardment, or microinjection.

A human producer cell line that stably expresses the necessary eVLP components in a constitutive and/or inducible fashion can be used for production of T2 and T4eVLPs. T2 and T4eVLPs can be produced from cells that have been derived from patients (autologous eVLPs) and other FDA-approved cell lines (allogenic eVLPs) if these cells have been converted into stable cell lines that express the aforementioned eVLP components.

Also provided herein are the producer cells themselves.

Production of Cargo-Loaded eVLPs and Compositions

Preferably eVLPs are harvested from cell culture medium supernatant 36-48 hours post-transfection, or when eVLPs are at the maximum concentration in the medium of the producer cells (the producer cells are expelling particles into the media and at some point in time, the particle concentration in the media will be optimal for harvesting the particles). Supernatant can be purified by any known methods in the art, such as centrifugation, ultracentrifugation, precipitation, ultrafiltration, and/or chromatography. In some embodiments, the supernatant is first filtered, e.g., to remove particles larger than 1 μm, e.g., through 0.45 pore size polyvinylidene fluoride hydrophilic membrane (Millipore Millex-HV) or 0.8 μm pore size mixed cellulose esters hydrophilic membrane (Millipore Millex-AA). After filtration, the supernatant can be further purified and concentrated, e.g., using ultracentrifugation, e.g., at a speed of 80,000 to 100,000×g at a temperature between 1° C. and 5° C. for 1 to 2 hours, or at a speed of 8,000 to 15,000 g at a temperature between 1° C. and 5° C. for 10 to 16 hours. After this centrifugation step, the eVLPs are concentrated in the form of a centrifugate (pellet), which can be resuspended to a desired concentration, mixed with transduction-enhancing reagents, subjected to a buffer exchange, or used as is. In some embodiments, eVLP-containing supernatant can be filtered, precipitated, centrifuged and resuspended to a concentrated solution. For example, polyethylene glycol (PEG), e.g., PEG 8000, or antibody-bead conjugates that bind to eVLP surface proteins or membrane components can be used to precipitate particles. Purified particles are stable and can be stored at 4° C. for up to a week or −80° C. for years without losing appreciable activity.

Preferably, eVLPs are resuspended or undergo buffer exchange so that particles are suspended in an appropriate carrier. In some embodiments, buffer exchange can be performed by ultrafiltration (Sartorius Vivaspin 500 MWCO 100,000). An exemplary appropriate carrier for eVLPs to be used for in vitro applications would preferably be a cell culture medium that is suitable for the cells that are to be transduced by eVLPs. Transduction-enhancing reagents that can be mixed into the purified and concentrated eVLP solution for in vitro applications include reagents known by those familiar with the art (Miltenyi Biotec Vectofusin-1, Millipore Polybrene, Takara Retronectin, Sigma Protamine Sulfate, and the like). After eVLPs in an appropriate carrier are applied to the cells to be transduced, transduction efficiency can be further increased by centrifugation. Preferably, the plate containing eVLPs applied to cells can be centrifuged at a speed of 1,150 g at room temperature for 30 minutes. After centrifugation, cells are returned into the appropriate cell culture incubator (humidified incubator at 37° C. with 5% CO₂).

An appropriate carrier for eVLPs to be administered to a mammal, especially a human, would preferably be a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions. Another appropriate pharmaceutical form would be aerosolized particles for administration by intranasal inhalation or intratracheal intubation.

The pharmaceutical forms suitable for injectable use include sterile aqueous solutions or suspensions. The solution or suspension may comprise additives which are compatible with eVLPs and do not prevent eVLP entry into target cells. In all cases, the form must be sterile and must be fluid to the extent that the form can be administered with a syringe. It must be stable under the conditions of manufacture and storage and must be preserved against the contaminating action of microorganisms, such as bacteria and fungi. An example of an appropriate solution is a buffer, such as phosphate buffered saline.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The compositions comprising cargo-loaded eVLPs can be included in a container, pack, or dispenser together with instructions for administration.

### Examples

The invention is further described in the following examples, which do not limit the scope of the invention described in the claims.

### Methods

The following methods were used in the Examples below. eVLP particles were produced by HEK293T cells using polyethylenimine (PEI) based transfection of plasmids. PEI is Polyethylenimine 25kD linear (Polysciences #23966-2). To make a stock ‘PEI MAX’ solution, lg of PEI was added to 1L endotoxin-free dH₂O that was previously heated to ˜80° C. and cooled to room temperature. This mixture was neutralized to pH 7.1 by addition of ION NaOH and filter sterilized with 0.22 μm polyethersulfone (PES). PEI MAX is stored at −20° C.

HEK293T cells were split to reach a confluency of 70%-90% at time of transfection and are cultured in 10% FBS DMEM media. Cargo vectors, such as one encoding a CMV promoter driving expression of a hPLCδ1 PH fusion to codon optimized Cas9 were co-transfected with a U6 promoter-sgRNA encoding plasmid and the VSV-G envelope plasmid pMD2.G (Addgene #12259). Transfection reactions were assembled in reduced serum media (Opti-MEM; GIBCO #31985-070). For eVLP particle production on 10 cm plates, 7.5 μg PH-Cas9 expressing plasmid, 7.5 μg sgRNA-expression plasmid and 5 μg pMD2.G were mixed in 1 mL Opti-MEM, followed by addition of 27.5 μl PEI MAX. After 20-30 min incubation at room temperature, the transfection reactions were dispersed dropwise over the HEK293T cells. eVLPs were harvested at 48-72 hours post-transfection. eVLP supernatants were filtered using 0.45 μm cellulose acetate or 0.8 μm PES membrane filters and transferred to polypropylene Beckman ultracentrifuge tubes that are used with the SW28 rotor (Beckman Coulter #326823). Each ultracentrifuge tube is filled with eVLP-containing supernatant from 3 10 cm plates to reach an approximate final volume of 35-37.5 ml. eVLP supernatant underwent ultracentrifugation at approximately 100,000×g, or 25,000 rpm, at 4° C. for 2 hours. After ultracentrifugation, supernatants were decanted and eVLP pellets resuspended in DMEM 10% FBS media such that they are now approximately 1,000 times more concentrated than they were before ultracentrifugation. eVLPs were added dropwise to cells that were seeded in a 24-well plate 24 hours prior to transduction. Polybrene (5-10 μg/mL in cell culture medium; Sigma-Aldrich #TR-1003-G) was supplemented to enhance transduction efficiency, if necessary. Vectofusin-1 (10 μg/mL in cell culture medium, Miltenyi Biotec #130-111-163) was supplemented to enhance transduction efficiency, if necessary. Immediately following the addition of eVLPs, the 24-well plate was centrifuged at 1,150×g for 30 min at room temperature to enhance transduction efficiency, if necessary.

### Example 1

Cas9 RNP was delivered in VSVG-pseudotyped VLPs with or without a fusion to a PH domain. T2eVLPs containing Cas9 with or without PH fusion and VEGF-targeting sgRNA were applied to HepG2, U2OS, HEK293T, CFPAC1, BeWo, Jurkat, K562, and primary T cells for 48 hours. Gene modification frequencies of the target site within VEGF were obtained by amplicon sequencing. FIG. 3 demonstrates that fusion to PH domains from hPLCδ1 or hAKT1 significantly enhanced delivery/editing efficiency of Cas9 in T2eVLPs.

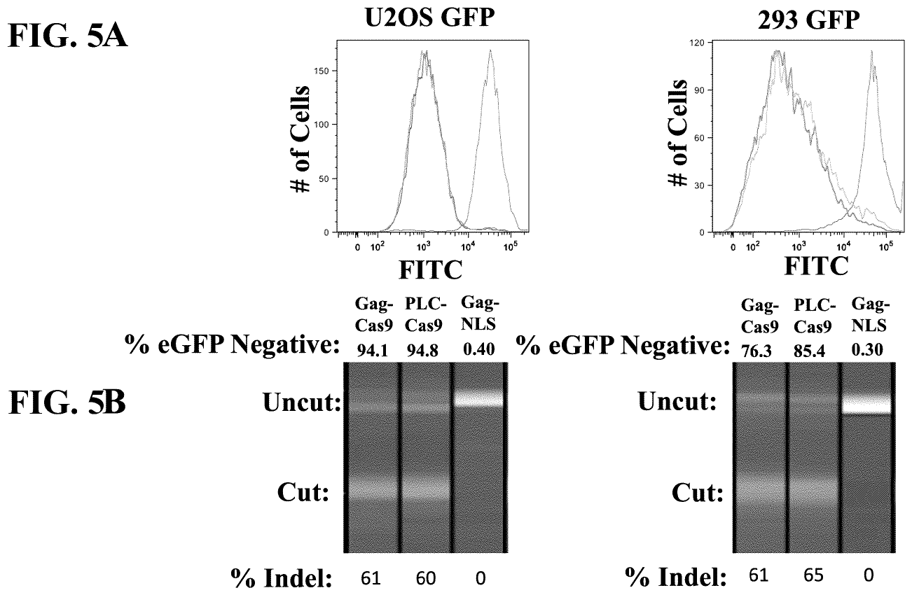

Gag fusions to Cas9 or PH fusions to Cas9 with guide RNA targeting GFP were packaged in VLPs or T1eVLPs, respectively. U2OS or HEK293 cell line stably expressing a single copy of GFP were treated with these particles that were previously purified from HEK293T cell culture media (DMEM) 48 hours after transfection of VSVG, Cas9 fusions and guide RNA expressing plasmids. Particle purification and concentration was performed by PVDF filtration and ultracentrifugation at 100,000×g for 2 hours. Gene modification frequencies were determined by T7E1 and flow cytometry. The results are shown in FIGS. 5A-B.

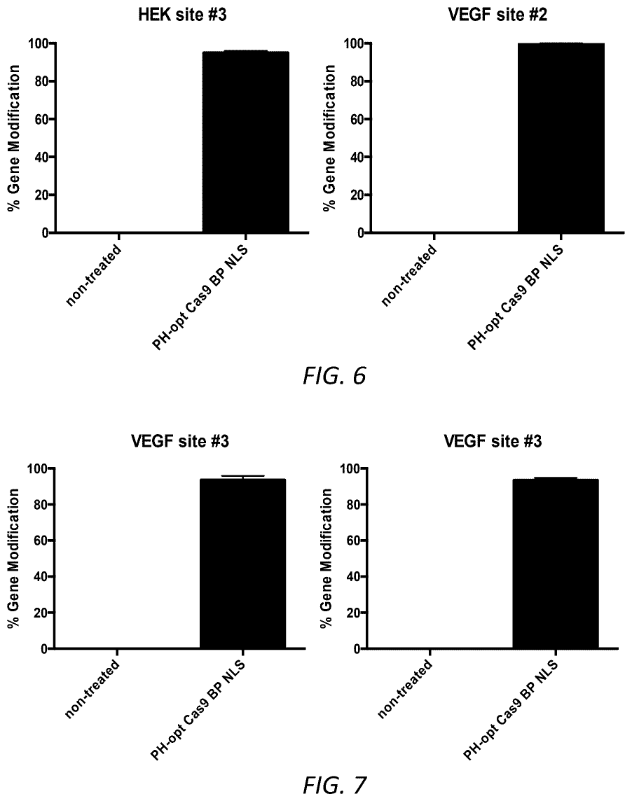

In FIG. 6, hPLCδ1 PH fusions to codon optimized Cas9 with guide RNA targeting HEK site #3 or VEGF site #2 were packaged in T1eVLPs. U2OS cells were treated with these particles that were previously purified from HEK293T cell culture media (DMEM, 10% FBS) 48 hours after transfection of VSVG, Cas9 fusions and guide RNA expressing plasmids. Particle purification and concentration was performed by filtration and ultracentrifugation at 100,000×g for 2 hours. Gene modification frequencies were determined by amplicon sequencing.

In FIG. 7, hPLCδ1 (left graph) or hAkt PH (right graph) fusions to codon optimized Cas9 with guide RNA targeting VEGF site #3 were packaged in T1eVLPs. U2OS cells were treated with these particles that were previously purified from HEK293T cell culture media (DMEM, 10% FBS) 48 hours after transfection of VSVG, Cas9 fusions and guide RNA expressing plasmids. Particle purification and concentration was performed by filtration and ultracentrifugation at 100,000×g for 2 hours. Gene modification frequencies were determined by amplicon sequencing.

In FIG. 8, gag fusions to the N or C terminus of Cas9-based base editors (BE3 and BE4) or PH fusions to the N or C terminus of BE3 and BE4 with guide RNA targeting VEGF site #2 were packaged in VLPs and eVLPs, respectively. HEK293T cells were treated with these particles that were previously purified from HEK293T cell culture media (DMEM) 48 hours after transfection of VSVG, base editor fusions and guide RNA expressing plasmids. Particle purification and concentration was performed by filtration and ultracentrifugation at 100,000×g for 2 hours. Gene modification frequencies were determined by amplicon sequencing.

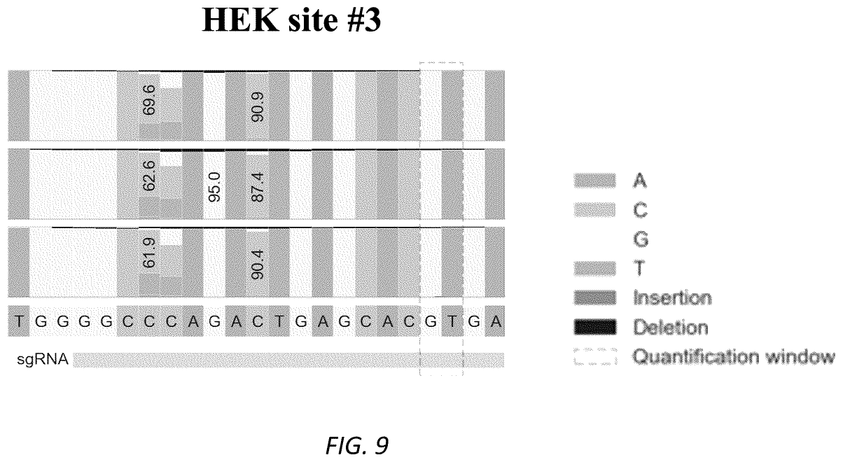

In FIG. 9, hPLCδ1 fusions to the N terminus of Cas9-based base editors (codon optimized BE4) with guide RNA targeting HEK site #3 were packaged in eVLPs. HEK293T cells were treated with these particles that were previously purified from HEK293T cell culture media (DMEM) 48 hours after transfection of VSVG, base editor fusions and guide RNA expressing plasmids. Particle purification and concentration was performed by filtration and ultracentrifugation at 100,000×g for 2 hours. Gene modification frequencies were determined by amplicon sequencing.

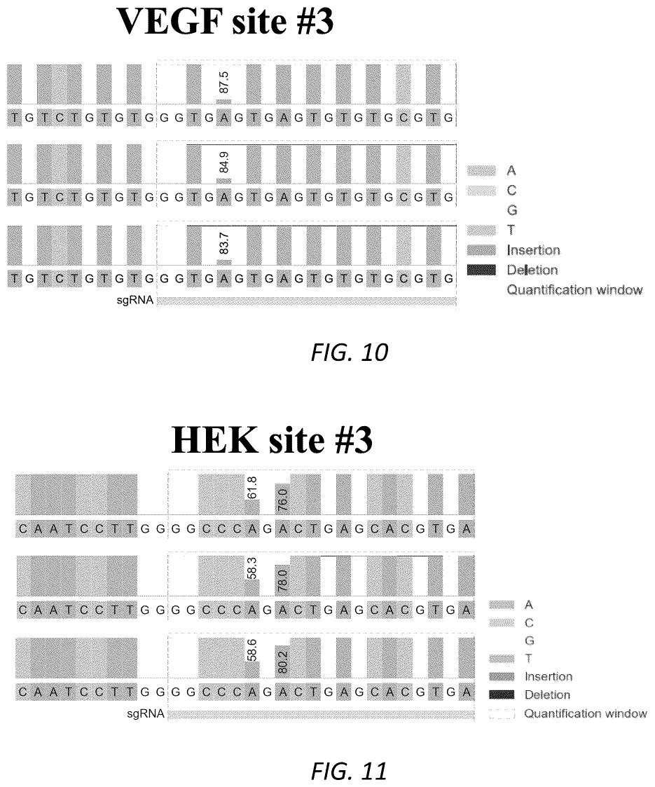

In FIG. 10, hPLCδ1 fusions to the N terminus of Cas9-based base editors (codon optimized ABE) with guide RNA targeting VEGF site #3 were packaged in eVLPs. HEK293T cells were treated with these particles that were previously purified from HEK293T cell culture media (DMEM) 48 hours after transfection of VSVG, base editor fusions and guide RNA expressing plasmids. Particle purification and concentration was performed by filtration and ultracentrifugation at 100,000×g for 2 hours. Gene modification frequencies were determined by amplicon sequencing.

In FIG. 11, hPLCδ1 fusions to the N terminus of Cas9-based base editors (codon optimized ABE) with guide RNA targeting HEK site #3 were packaged in eVLPs. HEK293T cells were treated with these particles that were previously purified from HEK293T cell culture media (DMEM) 48 hours after transfection of VSVG, base editor fusions and guide RNA expressing plasmids. Particle purification and concentration was performed by filtration and ultracentrifugation at 100,000×g for 2 hours. Gene modification frequencies were determined by amplicon sequencing.

In FIG. 12, gag fusions to Cas12a or hPLCδ1 PH fusions to Cas12a with guide RNA targeting FANCF site #1 were packaged in VLPs and eVLPs, respectively. HEK293 cells were treated with these particles that were previously purified from HEK293T cell culture media (DMEM) 48 hours after transfection of VSVG, Cas12a fusions and guide RNA expressing plasmids. Particle purification and concentration was performed by PVDF filtration and ultracentrifugation at 100,000×g for 2 hours. Gene modification frequencies were determined by T7E1.

In FIG. 13, hPLCδ1 PH fusions to MS2 with MS2-stem loop guide RNA targeting GFP site #1 were packaged in eVLPs with Cas9. HEK293 cells were treated with these particles that were previously purified from HEK293T cell culture media (DMEM) 48 hours after transfection of VSVG, Cas9, PH-MS2 fusions and MS2 stem loop guide RNA expressing plasmids. Particle purification and concentration was performed by PVDF filtration and ultracentrifugation at 100,000×g for 2 hours. Gene modification frequencies were determined by T7E1.

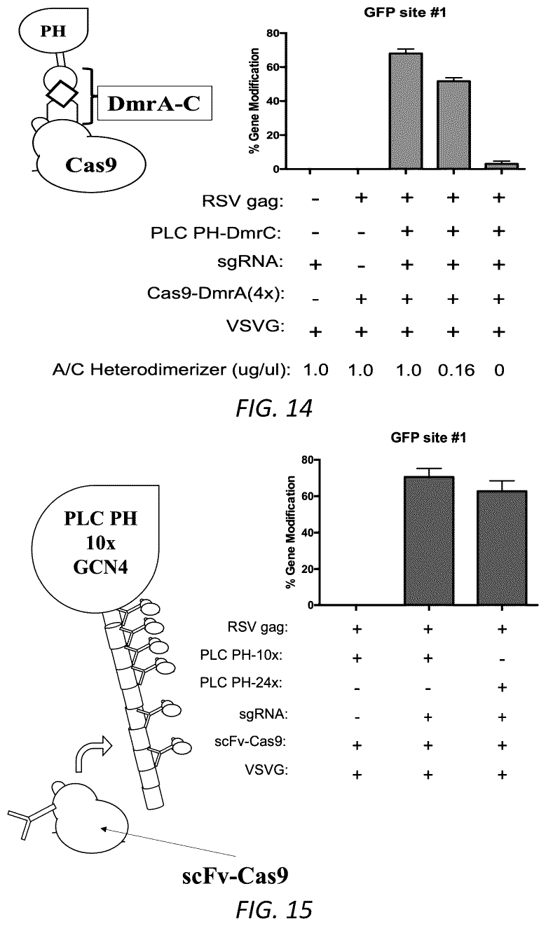

In FIG. 14, hPLCδ1 PH fusions to DmrC with guide RNA targeting GFP site #1 and Cas9 fused to DmrA repeats were packaged in eVLPs. HEK293 cells were treated with these particles that were previously purified from HEK293T cell culture media (DMEM) 48 hours after transfection of VSVG, DmrA-Cas9, PH-DmrC fusions and guide RNA expressing plasmids. Particle purification and concentration was performed by PVDF filtration and ultracentrifugation at 100,000×g for 2 hours. Gene modification frequencies were determined by T7E1.

In FIG. 15, hPLCδ1 PH fusions to GCN4 repeats with guide RNA targeting GFP site #1 and Cas9 fused to scFv were packaged in eVLPs. HEK293 cells were treated with these particles that were previously purified from HEK293T cell culture media (DMEM) 48 hours after transfection of VSVG, scFv-Cas9, PH-GCN4 fusions and guide RNA expressing plasmids. Particle purification and concentration was performed by PVDF filtration and ultracentrifugation at 100,000×g for 2 hours. Gene modification frequencies were determined by T7E1.

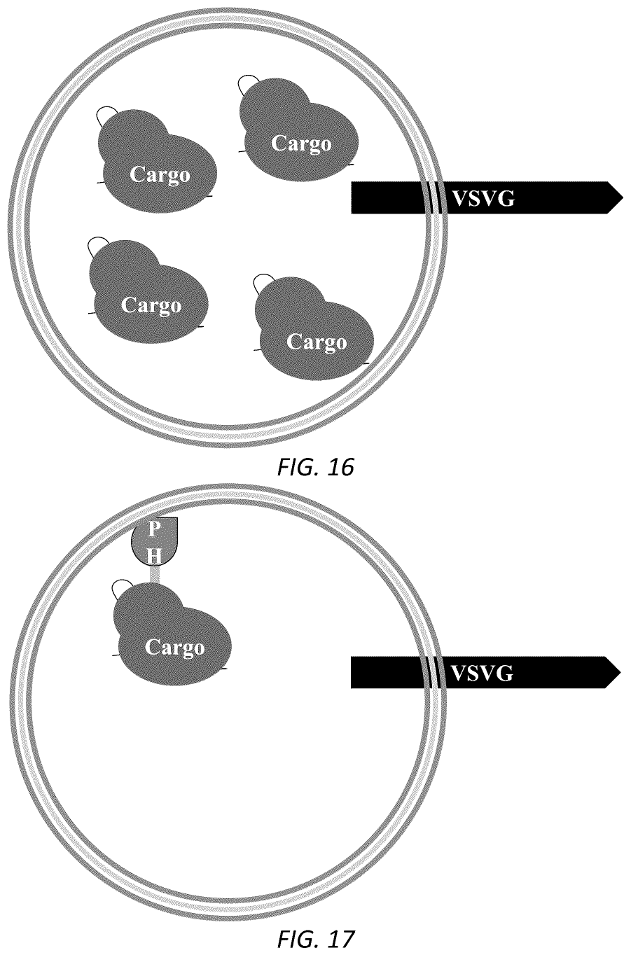

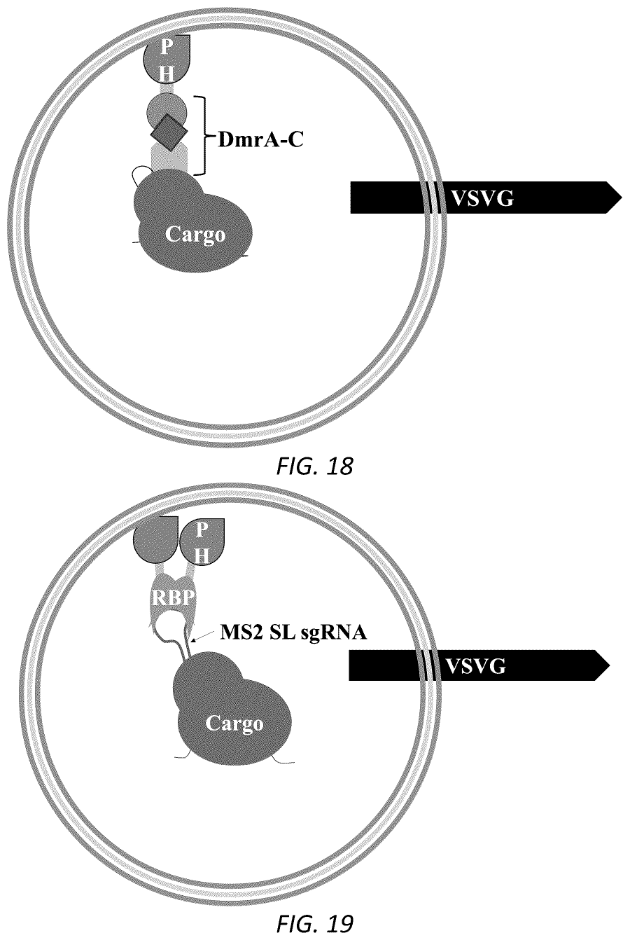

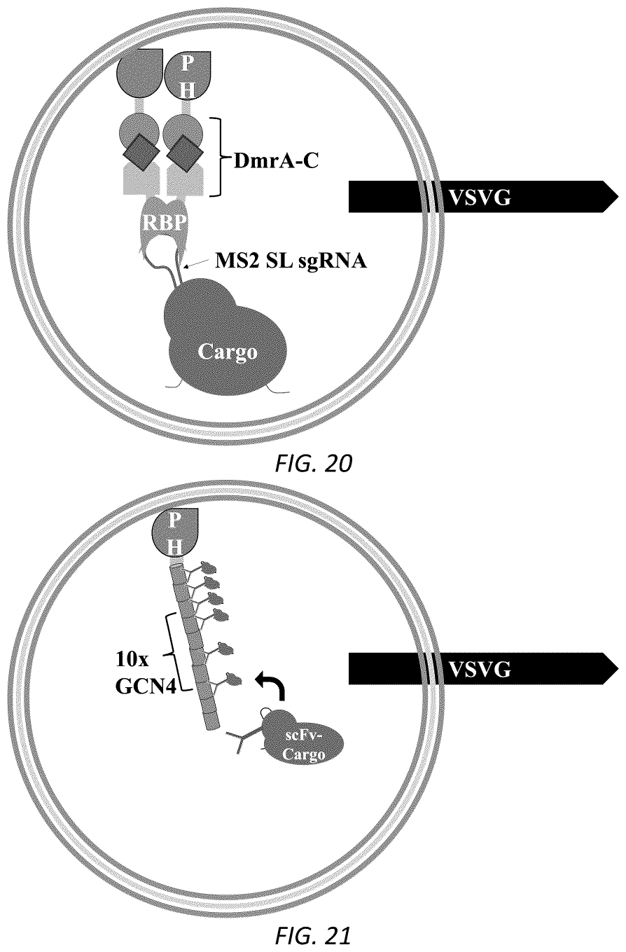

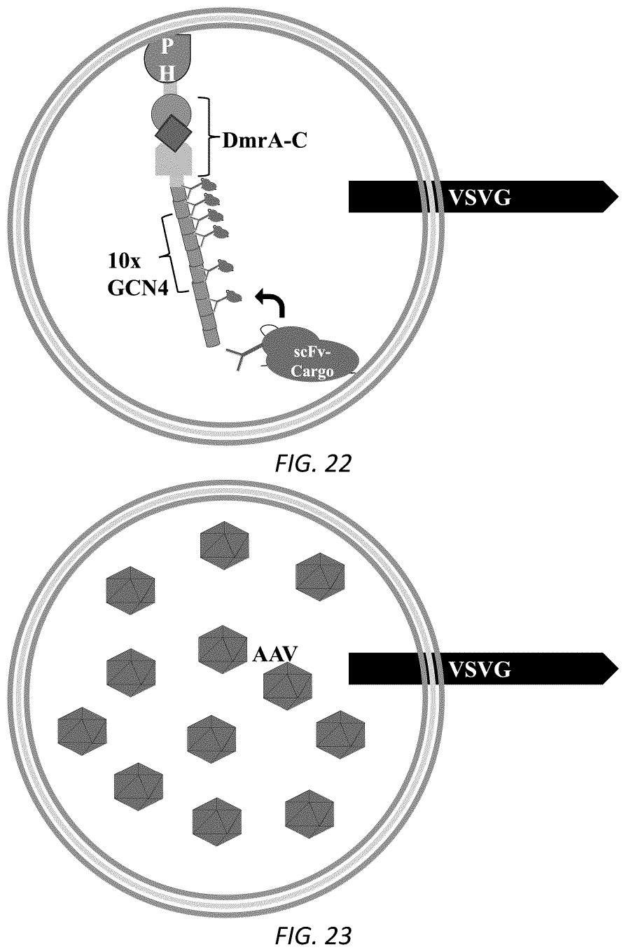

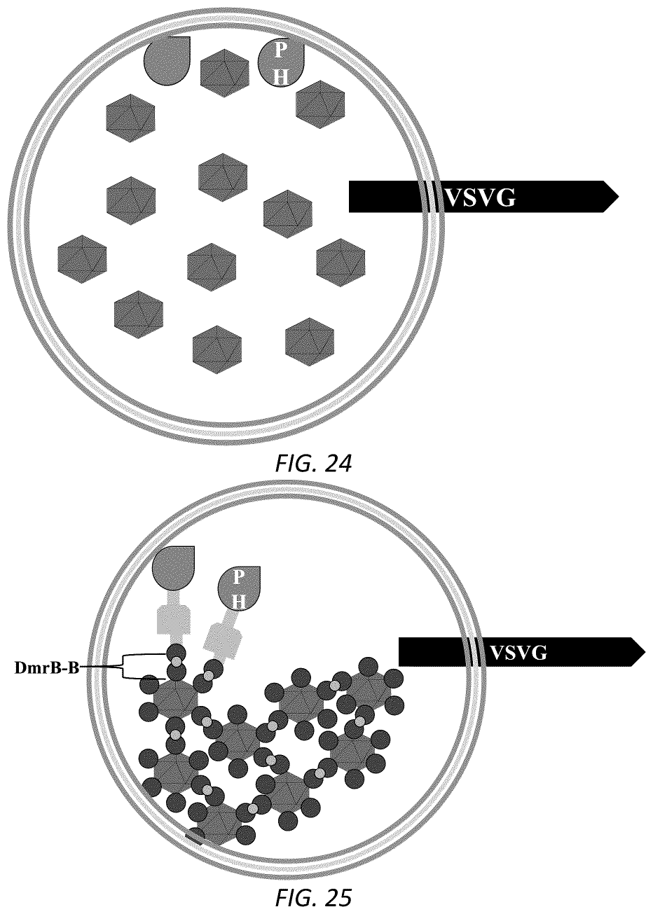

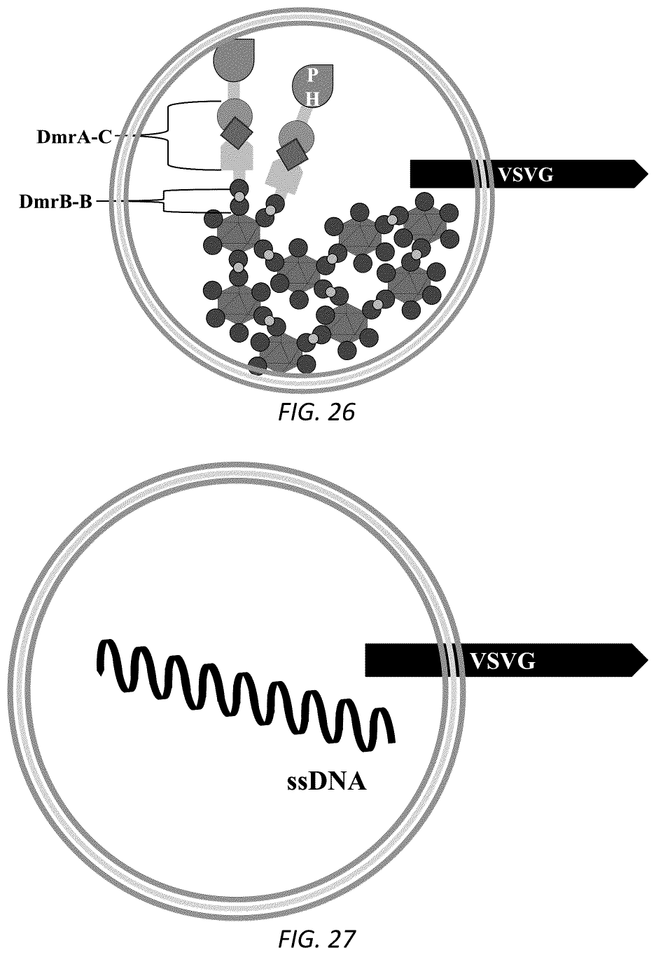

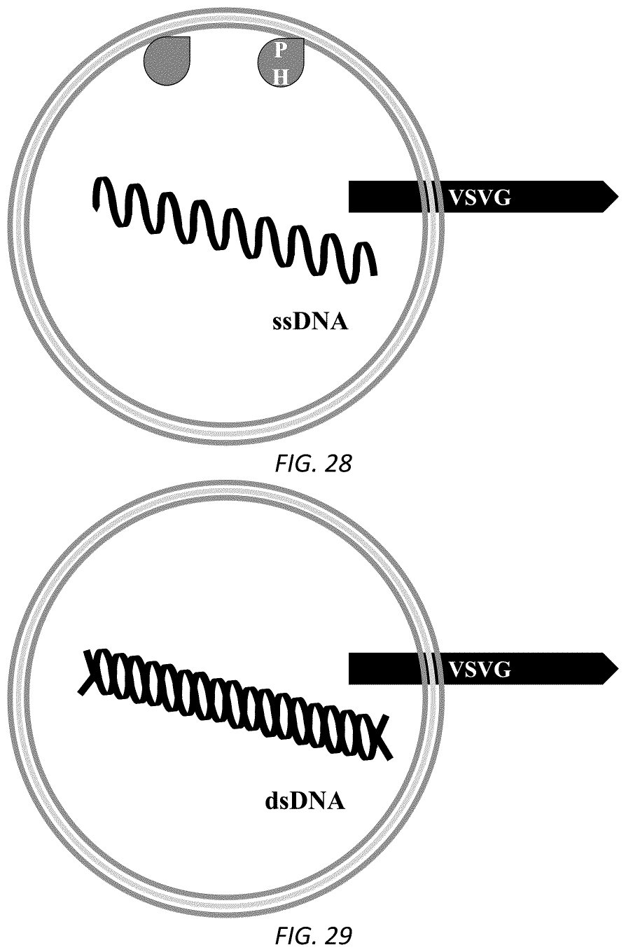

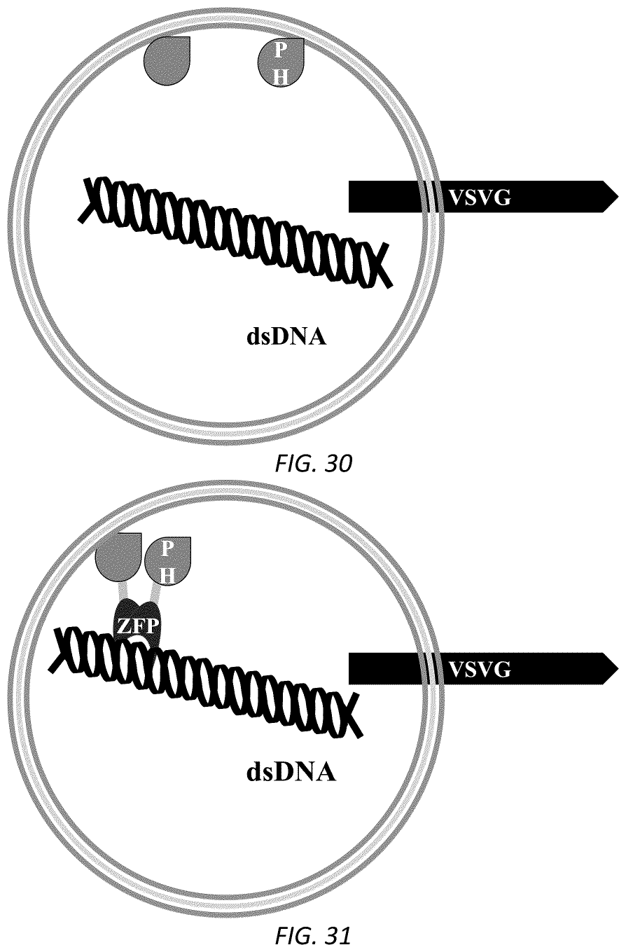

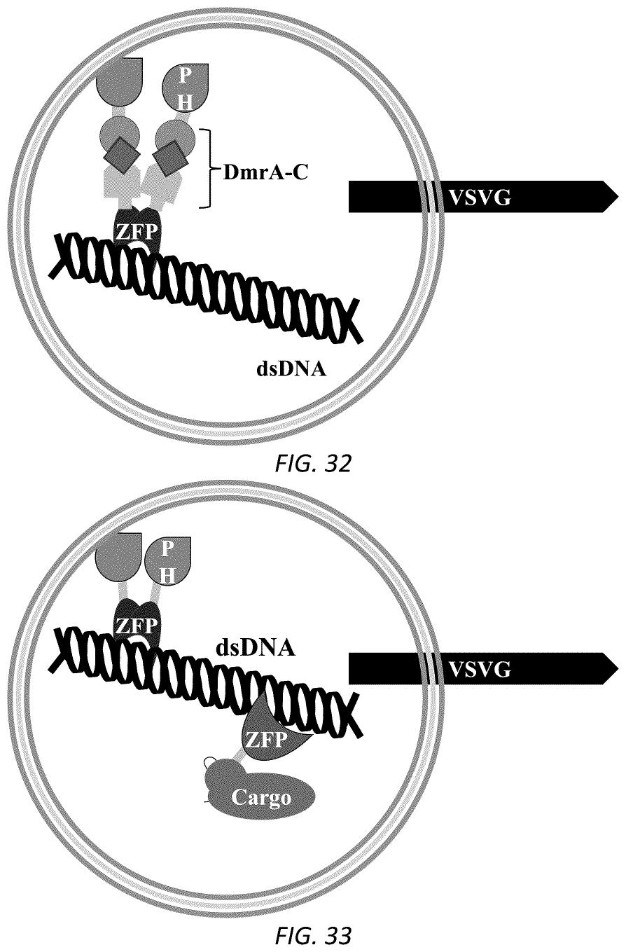

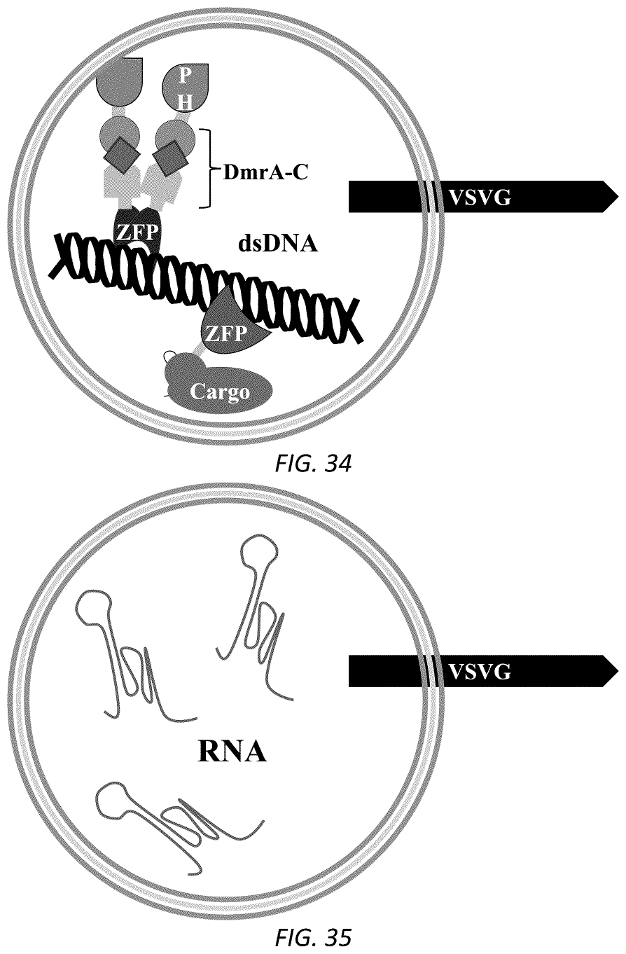

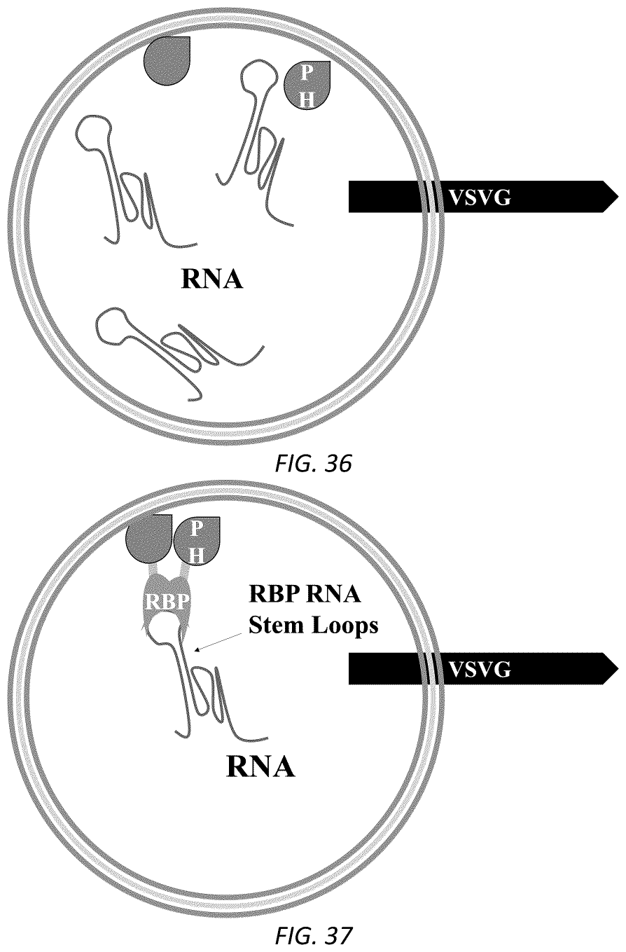

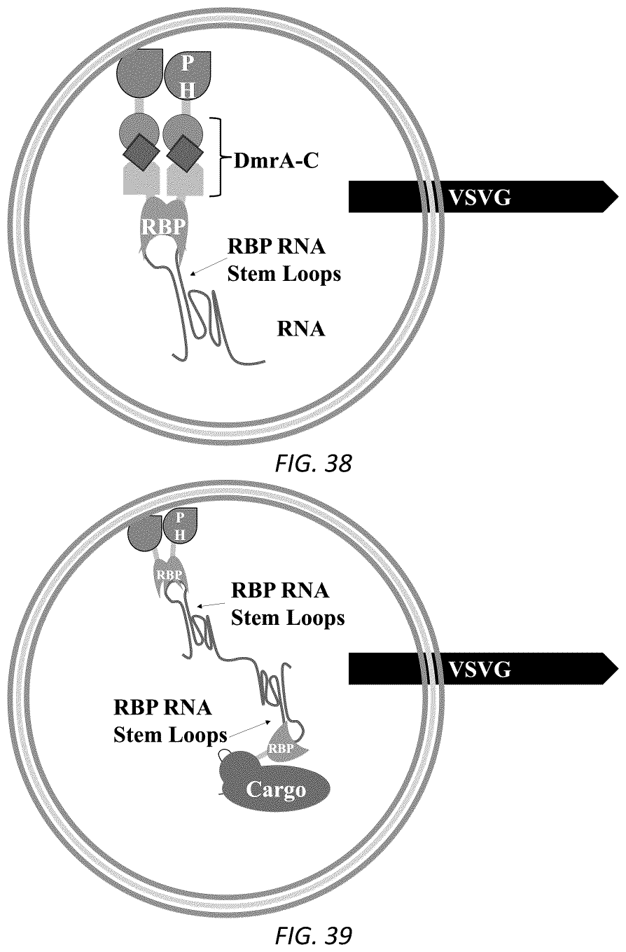

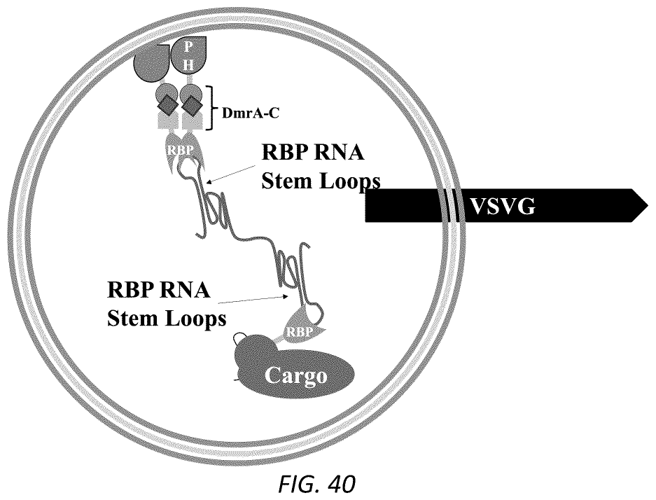

FIGS. 16-40 show various non-limiting examples of eVLP configurations and possible cargo.

TABLE 1
Exemplary Virally-derived glycoproteins.
Virally-derived glycoproteins

vesicular stomatitis virus glycoprotein (VSVG)
GP64
GP160
RD114
BaEVTR
BaEVTRless
FuG-E
FuG-E (P440E)
MLV ENV (ecotropic)
MLV ENV (amphotropic)
MLV 10A1

TABLE 2
Exemplary Potential Cas9 and Cas12a orthologs

| DNA-binding Cas | | | |
|---|---|---|---|
| ortholog | Enzyme class | Nickase mutation | CI mutations |
| SpCas9 | Type II-A | D10A | D10A, H840A |
| SaCas9 | Type II-A | D10A | D10A |
| CjCas9 | Type II-C | D8A | D8A |
| NmeCas9 | Type II-C | D16A | D16A, H588A |
| asCas12a | Type II-C | | D908A, E993A |
| lbCas12a | Type II-C | | D832A, E925A |

Nickase mutation residues represents a position of the enzyme either known to be required for catalytic activity of the conserved RuvC nuclease domain or predicted to be required for this catalytic activity based on sequence alignment to CjCas9 where structural information is lacking. All positional information refers to the wild-type protein sequences acquired from uniprot.org.

TABLE 3
Exemplary Deaminase domains and their
substrate sequence preferences.

| Deaminase | Nucleotide sequence preference |
|---|---|
| hAID | 5′-WRC |
| rAPOBEC1* | 5′-TC ≥ CC ≥ AC > GC |
| mAPOBEC3 | 5′-TYC |
| hAPOBEC3A | 5′-TCG |
| hAPOBEC3B | 5′-TCR > TCT |
| hAPOBEC3C | 5′-WYC |
| hAPOBEC3F | 5′-TTC |
| hAPOBEC3G | 5′-CCC |
| hAPOBEC3H | 5′-TTCA ~ TTCT ~ TTCG > |
| | ACCCA > TGCA |
| ecTadA | |
| hAdar1 | |
| hAdar2 | |

Nucleotide positions that are poorly specified or are permissive of two or more nucleotides are annotated according to IUPAC codes, where W = A or T, R = A or G, and Y = C or T.

TABLE 4
Exemplary Epigenetic modulator domains.

| Epigenetic modulator | Epigenetic modulation |
|---|---|
| VP16 | transcriptional activation |
| VP64 | transcriptional activation |
| P65 | transcriptional activation |
| RTA | transcriptional activation |
| KRAB | transcriptional repression |
| MeCP2 | transcriptional repression |
| Tet1 | Methylation |
| Dnmt3a | Methylation |

TABLE 5
Exemplary CRISPR based RNA-guided RNA binding enzymes

| RNA-binding Cas | |
|---|---|
| ortholog | Enzyme class |
| LshCas13a | Type-VI |
| LwaCas13a | Type-VI |
| PspCas13b | Type-VI |
| RfxCas13d | Type-VI |

TABLE 6
Exemplary plasma membrane recruitment domains

| # | Plasma membrane recruitment domain | Substitution(s) |
|---|---|---|
| 1. | Pleckstrin homology domain of human | |
| | phospholipase Cδ1 (hPLCδ1) | |
| 2. | Pleckstrin homology domain of human | |
| | Akt1 (hAktl) | |
| 3. | Mutant Pleckstrin homology domain of | E17K |
| | human Akt1 | |
| 4. | Pleckstrin homology domain of human | |
| | 3-phosphoinositide-dependent protein | |
| | kinase 1 (hPDPKI) | |
| 5. | Human CD9 | |
| 6. | Human CD47 | |
| 7. | Human CD63 | |
| 8. | Human CD81 | |
| 9. | Pleckstrin homology domain of Human Dapp1 | |
| 10. | Pleckstrin homology domain of Mouse Grp1 | |
| 11. | Pleckstrin homology domain of Human Grp1 | |
| 12. | Pleckstrin homology domain of Human OSBP | |
| 13. | Pleckstrin homology domain of Human Btk1 | |
| 14. | Pleckstrin homology domain of Human FAPP1 | |
| 15. | Pleckstrin homology domain of Human CERT | |
| 16. | Pleckstrin homology domain of Human PKD | |
| 17. | Pleckstrin homology domain of Human PHLPP1 | |
| 18. | Pleckstrin homology domain of Human SWAP70 | |
| 19. | Pleckstrin homology domain of Human MAPKAP1 | |

### Exemplary Relevant Protein Sequences:

Homo sapiens: Pleckstrin homology
domain of Human Dapp1
(SEQ ID NO: 1)
MQTGRTEDDLVPTAPSLGTKEGYLTKQGGLVKTWKTRWFTLHRNELK
YFKDQMSPEPIRILDLTECSAVQFDYSQERVNCFCLVFPFRTFYLCA
KTGVEADEWIKILRWKLSQIRKQLNQGEGTIR
Mus musculus: Pleckstrin homology domain
of Mouse Grp1
(SEQ ID NO: 2)
PFKIPEDDGNDLTHTFFNPDREGWLLKLGGRVKTWKRRWFILTDNCL
YYFEYTTDKEPRGIIPLENLSIREVEDPRKPNCFELYNPSHKGQVIK
ACKTEADGRVVEGNHVVYRISAPSPEEKEEWMKSIKASISRDPFYDM
LATRKRRIANKK
Homo sapiens: Pleckstrin homology
domain of Human Grp1
(SEQ ID NO: 3)
NPDREGWLLKLGGGRVKTWKRRWFILTDNCLYYFEYTTDKEPRGIIP
LENLSIREVEDPRKPNCFELYNPSHKGQVIKACKTEADGRVVEGNHV
VYRISAPSPEEKEEWMKSIKASIS
Homo sapiens: Pleckstrin homology
domain of Human OSBP
(SEQ ID NO: 4)
SGSAREGWLFKWTNYIKGYQRRWFVLSNGLLSYYRSKAEMRHTCRGT
INLATANITVEDSCNFIISNGGAQTYHLKASSEVERQRWVTALELAK
AKAVK
Homo sapiens: Pleckstrin homology
domain of Human Btk1
(SEQ ID NO: 5)
MAAVILESIFLKRSQQKKKTSPLNFKKRLFLLTVHKLSYYEYDFERG
RRGSKKGSIDVEKITCVETVVPEKNPPPERQIPRRGEESSEMEQISI
IERFPYPFQVVYDEGPLYVFSPTEELRKRWIHQLKNVIRYNSDLVQK
YHPCFWIDGQYLCCSQTAKNAMGCQILENRNGSLKP
Homo sapiens: Pleckstrin homology
domain of Human FAPP1
(SEQ ID NO: 6)
MEGVLYKWTNYLTGWQPRWFVLDNGILSYYDSQDDVCKGSKGSIKMA
VCEIKVHSADNTRMELIIPGEQHFYMKAVNAAERQRWLVALGSSKAC
LTDT
Homo sapiens: Pleckstrin homology
domain of Human CERT
(SEQ ID NO: 7)
MSDNQSWNSSGSEEDPETESGPPVERCGVLSKWTNYIHGWQDRWVLK
NNALSYYKSEDETEYGCRGSICLSKAVITPHDFDECRFDISVNDSVW
YLRAQDPDHRQQWIDAIEQHKTESGYG
Homo sapiens: Pleckstrin homology
domain of Human PKD
(SEQ ID NO: 8)
TVMKEGWMVHYTSKDTLRKRHYWRLDSKCITLFQNDTGSRYYKEIPL
SEILSLEPVKTSALIPNGANPHCFEITTANVVYYVGENVVNPSSPSP
NNSVLTSGVGADVARMWEIAIQHALM
Homo sapiens: Pleckstrin homology
domain of Human PHLPP1
(SEQ ID NO: 9)
RIQLSGMYNVRKGKMQLPVNRWTRRQVILCGTCLIVSSVKDSLTGKM
HVLPLIGGKVEEVKKHQHCLAFSSSGPQSQTYYICFDTFTEYLRWLR
QVSKVAS
Homo sapiens: Pleckstrin homology
domain of Human SWAP70
(SEQ ID NO: 10)
DVLKQGYMMKKGHRRKNWTERWFVLKPNIISYYVSEDLKDKKGDILL
DENCCVESLPDKDGKKCLFLVKCFDKTFEISASDKKKKQEWIQAIHS
TIH
Homo sapiens: Pleckstrin homology
domain of Human MAPKAP1
(SEQ ID NO: 11)
DMLSSHHYKSFKVSMIHRLRFTTDVQLGISGDKVEIDPVTNQKASTK
FWIKQKPISIDSDLLCACDLAEEKSPSHAIFKLTYLSNHDYKHLYFE
SDAATVNEIVLKVNYILES
Baboon Endogenous Retrovirus glycoprotein
(BaEVTR)
(SEQ ID NO: 12)
MGFTTKIIFLYNLVLVYAGFDDPRKAIELVQKRYGRPCDCSGGQVSE
PPSDRVSQVTCSGKTAYLMPDQRWKCKSIPKDTSPSGPLQECPCNSY
QSSVHSSCYTSYQQCRSGNKTYYTATLLKTQTGGTSDVQVLGSTNKL
IQSPCNGIKGQSICWSTTAPIHVSDGGGPLDTTRIKSVQRKLEEIHK
ALYPELQYHPLAIPKVRDNLMVDAQTLNILNATYNLLLMSNTSLVDD
CWLCLKLGPPTPLAIPNFLLSYVTRSSDNISCLIIPPLLVQPMQFSN
SSCLFSPSYNSTEEIDLGHVAFSNCTSITNVTGPICAVNGSVFLCGN
NMAYTYLPTNWTGLCVLATLLPDIDIIPGDEPVPIPAIDHFIYRPKR
AIQFIPLLAGLGITAAFTTGATGLGVSVTQYTKLSNQLISDVQILSS
TIQDLQDQVDSLAEVVLQNRRGLDLLTAEQGGICLALQEKCCFYVNK
SGIVRDKIKTLQEELERRRKDLASNPLWTGLQGLLPYLLPFLGPLLT
LLLLLTIGPCIFNRLVQFVKDRISVVQALVLTQQYHQLKPLEYEP
Modified Baboon Endogenous Retrovirus
glycoprotein (BaEVTRless)
(SEQ ID NO: 13)
MGFTTKIIFLYNLVLVYAGFDDPRKAIELVQKRYGRPCDCSGGQVSE
PPSDRVSQVTCSGKTAYLMPDQRWKCKSIPKDTSPSGPLQECPCNSY
QSSVHSSCYTSYQQCRSGNKTYYTATLLKTQTGGTSDVQVLGSTNKL
IQSPCNGIKGQSICWSTTAPIHVSDGGGPLDTTRIKSVQRKLEEIHK
ALYPELQYHPLAIPKVRDNLMVDAQTLNILNATYNLLLMSNTSLVDD
CWLCLKLGPPTPLAIPNFLLSYVTRSSDNISCLIIPPLLVQPMQFSN
SSCLFSPSYNSTEEIDLGHVAFSNCTSITNVTGPICAVNGSVFLCGN
NMAYTYLPTNWTGLCVLATLLPDIDIIPGDEPVPIPAIDHFIYRPKR
AIQFIPLLAGLGITAAFTTGATGLGVSVTQYTKLSNQLISDVQILSS
TIQDLQDQVDSLAEVVLQNRRGLDLLTAEQGGICLALQEKCCFYVNK
SGIVRDKIKTLQEELERRRKDLASNPLWTGLQGLLPYLLPFLGPLLT
LLLLLTIGPCIFNRLTAFINDKLNIIHAM
Fusion protein of Vesicular stomatitis
Indiana virus and Rabies virus
Glycoproteins (FuG-E)
(SEQ ID NO: 14)
MVPQVLLFVLLLGFSLCFGKFPIYTIPDELGPWSPIDIHHLSCPNNL
WEDEGCTNLSEFSYMELKVGYISAIKVNGFTCTGWTEAETYTNFVGY
VTTTFKRKHFRPTPDACRAAYNWKMAGDPRYEESLHNPYPDYHWLRT
VRTTKESLIIISPSVTDLDPYDKSLHSRVFPGGKCSGITVSSTYCST
NHDYTIWMPENPRPRTPCDIFTNSRGKRASNGNKTCGFVDERGLYKS
LKGACRLKLCGVLGLRLMDGTWVAMQTSDETKWCPPDQLVNLHDFRS
DEIEHLVVEELVKKREECLDALESIMTTKSVSFRRLSHLRKLVPGFG
KAYTIFNKTLMEADAHYKSVRTWNEIIPSKGCLKVGGRCHPHVNGVF
FNGIILGPDDHVLIPEMQSSLLQQHMELLESSVIPLMHPLADPSTVF
KEGDEAEDFVEVHLPKNPIELVEGWFSSWKSSIASFFFIIGLIIGLF
LVLRVGIHLCIKLKHTKKRQIYTDIEMNRLGK
Modified Fusion protein of Vesicular stomatitis
Indiana virus and Rabies virus
Glycoproteins (FuG-E (P440E))
(SEQ ID NO: 15)
MVPQVLLFVLLLGFSLCFGKFPIYTIPDELGPWSPIDIHHLSCPNNL
VVEDEGCTNLSEFSYMELKVGYISAIKVNGFTCTGVVTEAETYTNFV
GYVTTTFKRKHFRPTPDACRAAYNWKMAGDPRYEESLHNPYPDYHWL
RTVRTTKESLIIISPSVTDLDPYDKSLHSRVFPGGKCSGITVSSTYC
STNHDYTIWMPENPRPRTPCDIFTNSRGKRASNGNKTCGFVDERGLY
KSLKGACRLKLCGVLGLRLMDGTWVAMQTSDETKWCPPDQLVNLHDF
RSDEIEHLVVEELVKKREECLDALESIMTTKSVSFRRLSHLRKLVPG
FGKAYTIFNKTLMEADAHYKSVRTWNEIIPSKGCLKVGGRCHPHVNG
VFFNGIILGPDDHVLIPEMQSSLLQQHMELLESSVIPLMHPLADPST
VFKEGDEAEDFVEVHLEKNPIELVEGWFSSWKSSIASFFFIIGLIIG
LFLVLRVGIHLCIKLKHTKKRQIYTDIEMNRLGK
Amphotrophic Murine Leukemia Virus
(MLV ENV (amphotropic))
(SEQ ID NO: 16)
MARSTLSKPPQDKINPWKPLIVMGVLLGVGMAESPHQVFNVTWRVTN
LMTGRTANATSLLGTVQDAFPKLYFDLCDLVGEEWDPSDQEPYVGYG
CKYPAGRQRTRTFDFYVCPGHTVKSGCGGPGEGYCGKWGCETTGQAY
WKPTSSWDLISLKRGNTPWDTGCSKVACGPCYDLSKVSNSFQGATRG
GRCNPLVLEFTDAGKKANWDGPKSWGLRLYRTGTDPITMFSLTRQVL
NVGPRVPIGPNPVLPDQRLPSSPIEIVPAPQPPSPLNTSYPPSTTST
PSTSPTSPSVPQPPPGTGDRLLALVKGAYQALNLTNPDKTQECWLCL
VSGPPYYEGVAVVGTYTNHSTAPANCTATSQHKLTLSEVTGQGLCMG
AVPKTHQALCNTTQSAGSGSYYLAAPAGTMWACSTGLTPCLSTTVLN
LTTDYCVLVELWPRVIYHSPDYMYGQLEQRTKYKREPVSLTLALLLG
GLTMGGIAAGIGTGTTALIKTQQFEQLHAAIQTDLNEVEKSITNLEK
SLTSLSEVVLQNRRGLDLLFLKEGGLCAALKEECCFYADHTGLVRDS
MAKLRERLNQRQKLFETGQGWFEGLFNRSPWFTTLISTIMGPLIVLL
LILLFGPCILNRLVQFVKDRISVVQALVLTQQYHQLKPIEYEP
Ecotrophic Murine Leukemia Virus
(MLV ENV (Ecotropic))
(SEQ ID NO: 17)
MARSTLSKPLKNKVNPRGPLIPLILLMLRGVSTASPGSSPHQVYNIT
WEVTNGDRETVWATSGNHPLWTWWPDLTPDLCMLAHHGPSYWGLEYQ
SPFSSPPGPPCCSGGSSPGCSRDCEEPLTSLTPRCNTAWNRLKLDQT
THKSNEGFYVCPGPHRPRESKSCGGPDSFYCAYWGCETTGRAYWKPS
SSWDFITVNNNLTSDQAVQVCKDNKWCNPLVIRFTDAGRRVTSWTTG
HYWGLRLYVSGQDPGLTFGIRLRYQNLGPRVPIGPNPVLADQQPLSK
PKPVKSPSVTKPPSGTPLSPTQLPPAGTENRLLNLVDGAYQALNLTS
PDKTQECWLCLVAGPPYYEGVAVLGTYSNHTSAPANCSVASQHKLTL
SEVTGQGLCIGAVPKTHQALCNTTQTSSRGSYYLVAPTGTMWACSTG
LTPCISTTILNLTTDYCVLVELWPRVTYHSPSYVYGLFERSNRHKRE
PVSLTLALLLGGLTMGGIAAGIGTGTTALMATQQFQQLQAAVQDDLR
EVEKSISNLEKSLTSLSEVVLQNRRGLDLLFLKEGGLCAALKEECCF
YADHTGLVRDSMAKLRERLNQRQKLFESTQGWFEGLFNRSPWFTTLI
STIMGPLIVLLMILLFGPCILNRLVQFVKDRISVVQALVLTQQYHQL
KPIEYEP
Moloney murine leukemia virus 10A1
strain Glycoprotein (MLV 10A1)
(SEQ ID NO: 18)
MARSTLSKPLKDKINPWKSLMVMGVLLRVGMAESPHQVFNVTWRVTN
LMTGRTANATSLLGTVQDAFPRLYFDLCDLVGEEWDPSDQEPYVGYG
CKYPGGRKRTRTFDFYVCPGHTVKSGCGGPREGYCGEWGCETTGQAY
WKPTSSWDLISLKRGNTPWDTGCSKMACGPCYDLSKVSNSFQGATRG
GRCNPLVLEFTDAGKKANWDGPKSWGLRLYRTGTDPITMFSLTRQVL
NIGPRIPIGPNPVITGQLPPSRPVQIRLPRPPQPPPTGAASIVPETA
PPSQQPGTGDRLLNLVEGAYRALNLTNPDKTQECWLCLVSGPPYYEG
VAVVGTYTNHSTAPASCTATSQHKLTLSEVTGQGLCMGAVPKTHQAL
CNTTQSAGSGSYYLAAPAGTMWACSTGLTPCLSTTMLNLTTDYCVLV
ELWPRIIYHSPDYMYGQLEQRTKYKREPVSLTLALLLGGLTMGGIAA
GIGTGTTALIKTQQFEQLHAAIQTDLNEVEKSITNLEKSLTSLSEVV
LQNRRGLDLLFLKEGGLCAALKEECCFYADHTGLVRDSMAKLRERLN
QRQKLFESGQGWFEGLFNRSPWFTTLISTIMGPLIVLLLILLFGPCI
LNRLVQFVKDRISVVQALVLTQQYHQLKPIEYEP
Rattus norvegicus & synthetic: APOBEC1-XTEN
L8-nspCas9-UGI-SV40 NLS
(SEQ ID NO: 19)
MSSETGPVAVDPTLRRRIEPHEFEVFFDPRELRKETCLLYEINWGGR
HSIWRHTSQNTNKHVEVNFIEKFTTERYFCPNTRCSITWFLSWSPCG
ECSRAITEFLSRYPHVTLFIYIARLYHHADPRNRQGLRDLISSGVTI
QIMTEQESGYCWRNFVNYSPSNEAHWPRYPHLWVRLYVLELYCIILG
LPPCLNILRRKQPQLTFFTIALQSCHYQRLPPHILWATGLKSGSETP
GTSESATPESDKKYSIGLAIGTNSVGWAVITDEYKVPSKKFKVLGNT
DRHSIKKNLIGALLFDSGETAEATRLKRTARRRYTRRKNRICYLQEI
FSNEMAKVDDSFFHRLEESFLVEEDKKHERHPIFGNIVDEVAYHEKY
PTIYHLRKKLVDSTDKADLRLIYLALAHMIKFRGHFLIEGDLNPDNS
DVDKLFIQLVQTYNQLFEENPINASGVDAKAILSARLSKSRRLENLI
AQLPGEKKNGLFGNLIALSLGLTPNFKSNFDLAEDAKLQLSKDTYDD
DLDNLLAQIGDQYADLFLAAKNLSDAILLSDILRVNTEITKAPLSAS
MIKRYDEHHQDLTLLKALVRQQLPEKYKEIFFDQSKNGYAGYIDGGA
SQEEFYKFIKPILEKMDGTEELLVKLNREDLLRKQRTFDNGSIPHQI
HLGELHAILRRQEDFYPFLKDNREKIEKILTFRIPYYVGPLARGNSR
FAWMTRKSEETITPWNFEEVVDKGASAQSFIERMTNFDKNLPNEKVL
PKHSLLYEYFTVYNELTKVKYVTEGMRKPAFLSGEQKKAIVDLLFKT
NRKVTVKQLKEDYFKKIECFDSVEISGVEDRFNASLGTYHDLLKIIK
DKDFLDNEENEDILEDIVLTLTLFEDREMIEERLKTYAHLFDDKVMK
QLKRRRYTGWGRLSRKLINGIRDKQSGKTILDFLKSDGFANRNFMQL
IHDDSLTFKEDIQKAQVSGQGDSLHEHIANLAGSPAIKKGILQTVKV
VDELVKVMGRHKPENIVIEMARENQTTQKGQKNSRERMKRIEEGIKE
LGSQILKEHPVENTQLQNEKLYLYYLQNGRDMYVDQELDINRLSDYD
VDHIVPQSFLKDDSIDNKVLTRSDKNRGKSDNVPSEEVVKKMKNYWR
QLLNAKLITQRKFDNLTKAERGGLSELDKAGFIKRQLVETRQITKHV
AQILDSRMNTKYDENDKLIREVKVITLKSKLVSDFRKDFQFYKVREI
NNYHHAHDAYLNAVVGTALIKKYPKLESEFVYGDYKVYDVRKMIAKS
EQEIGKATAKYFFYSNIMNFFKTEITLANGEIRKRPLIETNGETGEI
VWDKGRDFATVRKVLSMPQVNIVKKTEVQTGGFSKESILPKRNSDKL
IARKKDWDPKKYGGFDSPTVAYSVLVVAKVEKGKSKKLKSVKELLGI
TIMERSSFEKNPIDFLEAKGYKEVKKDLIIKLPKYSLFELENGRKRM
LASAGELQKGNELALPSKYVNFLYLASHYEKLKGSPEDNEQKQLFVE
QHKHYLDEIIEQISEFSKRVILADANLDKVLSAYNKHRDKPIREQAE
NIIHLFTLTNLGAPAAFKYFDTTIDRKRYTSTKEVLDATLIHQSITG
LYETRIDLSQLGGDSGGSTNLSDIIEKETGKQLVIQESILMLPEEVE
EVIGNKPESDILVHTAYDESTDENVMLLTSDAPEYKPWALVIQDSNG
ENKIKMLSGGSPKKKRKV
Homo sapiens: AID
(SEQ ID NO: 20)
MDSLLMNRRKFLYQFKNVRWAKGRRETYLCYVVKRRDSATSFSLDFG
YLRNKNGCHVELLFLRYISDWDLDPGRCYRVTWFTSWSPCYDCARHV
ADFLRGNPNLSLRIFTARLYFCEDRKAEPEGLRRLHRAGVQIAIMTF
KDYFYCWNTFVENHERTFKAWEGLHENSVRLSRQLRRILLPLYEVDD
LRDAFRTLGL
Homo sapiens: MDv solubility variant lacking
N-terminal RNA-binding region
(SEQ ID NO: 21)
LMDPHIFTSNFNNGIGRHKTYLCYEVERLDSATSFSLDFGYLRNKNG
CHVELLFLRYISDWDLDPGRCYRVTWFTSWSPCYDCARHVADFLRGN
PNLSLRIFTARLYFCEDRKAEPEGLRRLHRAGVQIAIMTFKDYFYCW
NTFVENHERTFKAWEGLHENSVRLSRQLRRILLPLYEVDDLRDAFRT
LGL
Homo sapiens: AIDv solubility variant
lacking N-terminal RNA-binding region and
the C-terminal poorly structured region
(SEQ ID NO: 22)
MDPHIFTSNFNNGIGRHKTYLCYEVERLDSATSFSLDFGYLRNKNGC
HVELLFLRYISDWDLDPGRCYRVTWFTSWSPCYDCARHVADFLRGNP
NLSLRIFTARLYFCEDRKAEPEGLRRLHRAGVQIAIMTFKDYFYCWN
TFVENHERTFKAWEGLHENSVRLSRQLRRILLPL
Rattus norvegicus: AP0BEC1
(SEQ ID NO: 23)
MSSETGPVAVDPTLRRRIEPHEFEVFFDPRELRKETCLLYEINWGGR
HSIWRHTSQNTNKHVEVNFIEKFTTERYFCPNTRCSITWFLSWSPCG
ECSRAITEFLSRYPHVTLFIYIARLYHHADPRNRQGLRDLISSGVTI
QIMTEQESGYCWRNFVNYSPSNEAHWPRYPHLWVRLYVLELYCIILG
LPPCLNILRRKQPQLTFFTIALQSCHYQRLPPHILWATGLK
Mus musculus: APOBEC3
(SEQ ID NO: 24)
MGPFCLGCSHRKCYSPIRNLISQETFKFHFKNLGYAKGRKDTFLCYE
VTRKDCDSPVSLHHGVFKNKDNIHAEICFLYWFHDKVLKVLSPREEF
KITWYMSWSPCFECAEQIVRFLATHHNLSLDIFSSRLYNVQDPETQQ
NLCRLVQEGAQVAAMDLYEFKKCWKKFVDNGGRRFRPWKRLLTNFRY
QDSKLQEILRRMDPLSEEEFYSQFYNQRVKHLCYYHRMKPYLCYQLE
QFNGQAPLKGCLLSEKGKQHAEILFLDKIRSMELSQVTITCYLTWSP
CPNCAWQLAAFKRDRPDLILHIYTSRLYFHWKRPFQKGLCSLWQSGI
LVDVMDLPQFTDCWTNFVNPKRPFRPWKGLEIISRRTQRRLRRIKES
WGLQDLVNDFGNLQLGPPMSN
Mus musculus: APOBEC3 catalytic domain
(SEQ ID NO: 25)
MGPFCLGCSHRKCYSPIRNLISQETFKFHFKNLGYAKGRKDTFLCYE
VTRKDCDSPVSLHHGVFKNKDNIHAEICFLYWFHDKVLKVLSPREEF
KITWYMSWSPCFECAEQIVRFLATHHNLSLDIFSSRLYNVQDPETQQ
NLCRLVQEGAQVAAMDLYEFKKCWKKFVDNGGRRFRPWKRLLTNFRY
QDSKLQEILRR
Homo sapiens: APOBEC3A
(SEQ ID NO: 26)
MEASPASGPRHLMDPHIFTSNFNNGIGRHKTYLCYEVERLDNGTSVK
MDQHRGFLHNQAKNLLCGFYGRHAELRFLDLVPSLQLDPAQIYRVTW
FISWSPCFSWGCAGEVRAFLQENTHVRLRIFAARIYDYDPLYKEALQ
MLRDAGAQVSIMTYDEFKHCWDTFVDHQGCPFQPWDGLDEHSQALSG
RLRAILQNQGN
Homo sapiens: APOBEC3G
(SEQ ID NO: 27)
MKPHFRNTVERMYRDTFSYNFYNRPILSRRNTVWLCYEVKTKGPSRP
PLDAKIFRGQVYSELKYHPEMRFFHWFSKWRKLHRDQEYEVTWYISW
SPCTKCTRDMATFLAEDPKVTLTIFVARLYYFWDPDYQEALRSLCQK
RDGPRATMKIMNYDEFQHCWSKFVYSQRELFEPWNNLPKYYILLHIM
LGEILRHSMDPPTFTFNFNNEPWVRGRHETYLCYEVERMHNDTWVLL
NQRRGFLCNQAPHKHGFLEGRHAELCFLDVIPFWKLDLDQDYRVTCF
TSWSPCFSCAQEMAKFISKNKHVSLCIFTARIYDDQGRCQEGLRTLA
EAGAKISIMTYSEFKHCWDTFVDHQGCPFQPWDGLDEHSQDLSGRLR
AILQNQEN
Homo sapiens: APOBEC3G catalytic domain
(SEQ ID NO: 28)
PPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCA
QEMAKFISKNKHVSLCIFTARIYDDQGRCQEGLRTLAEAGAKISIMT
YSEFKHCWDTFVDHQGCPFQPWDGLDEHSQDLSGRLRAILQNQEN
Homo sapiens: APOBEC3H
(SEQ ID NO: 29)
MALLTAETFRLQFNNKRRLRRPYYPRKALLCYQLTPQNGSTPTRGYF
ENKKKCHAEICFINEIKSMGLDETQCYQVTCYLTWSPCSSCAWELVD
FIKAHDHLNLGIFASRLYYHWCKPQQKGLRLLCGSQVPVEVMGFPKF
ADCWENFVDHEKPLSFNPYKMLEELDKNSRAIKRRLERIKIPGVRAQ
GRYMDILCDAEV
Homo sapiens: APOBEC3F
(SEQ ID NO: 30)
MKPHFRNTVERMYRDTFSYNFYNRPILSRRNTVWLCYEVKTKGPSRP
RLDAKIFRGQVYSQPEHHAEMCFLSWFCGNQLPAYKCFQITWFVSWT
PCPDCVAKLAEFLAEHPNVTLTISAARLYYYWERDYRRALCRLSQAG
ARVKIMDDEEFAYCWENFVYSEGQPFMPWYKFDDNYAFLHRTLKEIL
RNPMEAMYPHIFYFHFKNLRKAYGRNESWLCFTMEVVKHHSPVSWKR
GVFRNQVDPETHCHAERCFLSWFCDDILSPNTNYEVTWYTSWSPCPE
CAGEVAEFLARHSNVNLTIFTARLYYFWDTDYQEGLRSLSQEGASVE
IMGYKDFKYCWENFVYNDDEPFKPWKGLKYNFLFLDSKLQEILE
Homo sapiens: APOBEC3F catalytic domain
(SEQ ID NO: 31)
KEILRNPMEAMYPHIFYFHFKNLRKAYGRNESWLCFTMEVVKHHSPV
SWKRGVFRNQVDPETHCHAERCFLSWFCDDILSPNTNYEVTWYTSWS
PCPECAGEVAEFLARHSNVNLTIFTARLYYFWDTDYQEGLRSLSQEG
ASVEIMGYKDFKYCWENFVYNDDEPFKPWKGLKYNFLFLDSKLQEIL
E
Escherichia coli: TadA
(SEQ ID NO: 32)
MKRTADGSEFESPKKKRKVSEVEFSHEYWMRHALTLAKRAWDEREVP
VGAVLVHNNRVIGEGWNRPIGRHDPTAHAEIMALRQGGLVMQNYRLI
DATLYVTLEPCVMCAGAMIHSRIGRWFGARDAKTGAAGSLMDVLHHP
GMNHRVEITEGILADECAALLSDFFRMRRQEIKAQKKAQSSTDSGGS
SGGSSGSETPGTSESATPESSGGSSGGSSEVEFSHEYWMRHALTLAK
RARDEREVPVGAVLVLNNRVIGEGWNRAIGLHDPTAHAEIMALRQGG
LVMQNYRLIDATLYVTFEPCVMCAGAMIHSRIGRVVFGVRNAKTGAA
GSLMDVLHYPGMNHRVEITEGILADECAALLCYFFRMPRQVFNAQKK
AQSSTD
Homo sapiens: Adar1
(SEQ ID NO: 33)
MNPRQGYSLSGYYTHPFQGYEHRQLRYQQPGPGSSPSSFLLKQIEFL
KGQLPEAPVIGKQTPSLPPSLPGLRPRFPVLLASSTRGRQVDIRGVP
RGVHLGSQGLQRGFQHPSPRGRSLPQRGVDCLSSHFQELSIYQDQEQ
RILKFLEELGEGKATTAHDLSGKLGTPKKEINRVLYSLAKKGKLQKE
AGTPPLWKIAVSTQAWNQHSGVVRPDGHSQGAPNSDPSLEPEDRNST
SVSEDLLEPFIAVSAQAWNQHSGVVRPDSHSQGSPNSDPGLEPEDSN
STSALEDPLEFLDMAEIKEKICDYLFNVSDSSALNLAKNIGLTKARD
INAVLIDMERQGDVYRQGTTPPIWHLTDKKRERMQIKRNTNSVPETA
PAAIPETKRNAEFLTCNIPTSNASNNMVTTEKVENGQEPVIKLENRQ
EARPEPARLKPPVHYNGPSKAGYVDFENGQWATDDIPDDLNSIRAAP
GEFRAIMEMPSFYSHGLPRCSPYKKLTECQLKNPISGLLEYAQFASQ
TCEFNMIEQSGPPHEPRFKFQVVINGREFPPAEAGSKKVAKQDAAMK
AMTILLEEAKAKDSGKSEESSHYSTEKESEKTAESQTPTPSATSFFS
GKSPVTTLLECMHKLGNSCEFRLLSKEGPAHEPKFQYCVAVGAQTFP
SVSAPSKKVAKQMAAEEAMKALHGEATNSMASDNQPEGMISESLDNL
ESMMPNKVRKIGELVRYLNTNPVGGLLEYARSHGFAAEFKLVDQSGP
PHEPKFVYQAKVGGRWFPAVCAHSKKQGKQEAADAALRVLIGENEKA
ERMGFTEVTPVTGASLRRTMLLLSRSPEAQPKTLPLTGSTFHDQIAM
LSHRCFNTLTNSFQPSLLGRKILAAIIMKKDSEDMGVVVSLGTGNRC
VKGDSLSLKGETVNDCHAEIISRRGFIRFLYSELMKYNSQTAKDSIF
EPAKGGEKLQIKKTVSFHLYISTAPCGDGALFDKSCSDRAMESTESR
HYPVFENPKQGKLRTKVENGEGTIPVESSDIVPTWDGIRLGERLRTM
SCSDKILRWNVLGLQGALLTHFLQPIYLKSVTLGYLFSQGHLTRAIC
CRVTRDGSAFEDGLRHPFIVNHPKVGRVSIYDSKRQSGKTKETSVNW
CLADGYDLEILDGTRGTVDGPRNELSRVSKKNIFLLFKKLCSFRYRR
DLLRLSYGEAKKAARDYETAKNYFKKGLKDMGYGNWISKPQEEKNFY
LCPV
Streptococcus pyogenes: spCas9 Bipartite NLS
(SEQ ID NO: 34)
MDKKYSIGLDIGTNSVGWAVITDEYKVPSKKFKVLGNTDRHSIKKNL
IGALLFDSGETAEATRLKRTARRRYTRRKNRICYLQEIFSNEMAKVD
DSFFHRLEESFLVEEDKKHERHPIFGNIVDEVAYHEKYPTIYHLRKK
LVDSTDKADLRLIYLALAHMIKFRGHFLIEGDLNPDNSDVDKLFIQL
VQTYNQLFEENPINASGVDAKAILSARLSKSRRLENLIAQLPGEKKN
GLFGNLIALSLGLTPNFKSNFDLAEDAKLQLSKDTYDDDLDNLLAQI
GDQYADLFLAAKNLSDAILLSDILRVNTEITKAPLSASMIKRYDEHH
QDLTLLKALVRQQLPEKYKEIFFDQSKNGYAGYIDGGASQEEFYKFI
KPILEKMDGTEELLVKLNREDLLRKQRTFDNGSIPHQIHLGELHAIL
RRQEDFYPFLKDNREKIEKILTFRIPYYVGPLARGNSRFAWMTRKSE
ETITPWNFEEVVDKGASAQSFIERMTNFDKNLPNEKVLPKHSLLYEY
FTVYNELTKVKYVTEGMRKPAFLSGEQKKAIVDLLFKTNRKVTVKQL
KEDYFKKIECFDSVEISGVEDRFNASLGTYHDLLKIIKDKDFLDNEE
NEDILEDIVLTLTLFEDREMIEERLKTYAHLFDDKVMKQLKRRRYTG
WGRLSRKLINGIRDKQSGKTILDFLKSDGFANRNFMQLIHDDSLTFK
EDIQKAQVSGQGDSLHEHIANLAGSPAIKKGILQTVKVVDELVKVMG
RHKPENIVIEMARENQTTQKGQKNSRERMKRIEEGIKELGSQILKEH
PVENTQLQNEKLYLYYLQNGRDMYVDQELDINRLSDYDVDHIVPQSF
LKDDSIDNKVLTRSDKNRGKSDNVPSEEVVKKMKNYWRQLLNAKLIT
QRKFDNLTKAERGGLSELDKAGFIKRQLVETRQITKHVAQILDSRMN
TKYDENDKLIREVKVITLKSKLVSDFRKDFQFYKVREINNYHHAHDA
YLNAVVGTALIKKYPKLESEFVYGDYKVYDVRKMIAKSEQEIGKATA
KYFFYSNIMNFFKTEITLANGEIRKRPLIETNGETGEIVWDKGRDFA
TVRKVLSMPQVNIVKKTEVQTGGFSKESILPKRNSDKLIARKKDWDP
KKYGGFDSPTVAYSVLVVAKVEKGKSKKLKSVKELLGITIMERSSFE
KNPIDFLEAKGYKEVKKDLIIKLPKYSLFELENGRKRMLASAGELQK
GNELALPSKYVNFLYLASHYEKLKGSPEDNEQKQLFVEQHKHYLDEI
IEQISEFSKRVILADANLDKVLSAYNKHRDKPIREQAENIIHLFTLT
NLGAPAAFKYFDTTIDRKRYTSTKEVLDATLIHQSITGLYETRIDLS
QLGGDGSGGGGSGKRTADGSEFEPKKKRKVSSGGDYKDHDGDYKDHD
IDYKDDDDK
Staphylococcus aureus: saCas9
(SEQ ID NO: 35)
MKRNYILGLDIGITSVGYGIIDYETRDVIDAGVRLFKEANVENNEGR
RSKRGARRLKRRRRHRIQRVKKLLFDYNLLTDHSELSGINPYEARVK
GLSQKLSEEEFSAALLHLAKRRGVHNVNEVEEDTGNELSTKEQISRN
SKALEEKYVAELQLERLKKDGEVRGSINRFKTSDYVKEAKQLLKVQK
AYHQLDQSFIDTYIDLLETRRTYYEGPGEGSPFGWKDIKEWYEMLMG
HCTYFPEELRSVKYAYNADLYNALNDLNNLVITRDENEKLEYYEKFQ
IIENVFKQKKKPTLKQIAKEILVNEEDIKGYRVTSTGKPEFTNLKVY
HDIKDITARKEIIENAELLDQIAKILTIYQSSEDIQEELTNLNSELT
QEEIEQISNLKGYTGTHNLSLKAINLILDELWHTNDNQIAIFNRLKL
VPKKVDLSQQKEIPTTLVDDFILSPVVKRSFIQSIKVINAIIKKYGL
PNDIIIELAREKNSKDAQKMINEMQKRNRQTNERIEEIIRTTGKENA
KYLIEKIKLHDMQEGKCLYSLEAIPLEDLLNNPFNYEVDHIIPRSVS
FDNSFNNKVLVKQEENSKKGNRTPFQYLSSSDSKISYETFKKHILNL
AKGKGRISKTKKEYLLEERDINRFSVQKDFINRNLVDTRYATRGLMN
LLRSYFRVNNLDVKVKSINGGFTSFLRRKWKFKKERNKGYKHHAEDA
LIIANADFIFKEWKKLDKAKKVMENQMFEEKQAESMPEIETEQEYKE
IFITPHQIKHIKDFKDYKYSHRVDKKPNRELINDTLYSTRKDDKGNT
LIVNNLNGLYDKDNDKLKKLINKSPEKLLMYHHDPQTYQKLKLIMEQ
YGDEKNPLYKYYEETGNYLTKYSKKDNGPVIKKIKYYGNKLNAHLDI
TDDYPNSRNKVVKLSLKPYRFDVYLDNGVYKFVTVKNLDVIKKENYY
EVNSKCYEEAKKLKKISNQAEFIASFYNNDLIKINGELYRVIGVNND
LLNRIEVNMIDITYREYLENMNDKRPPRIIKTIASKTQSIKKYSTDI
LGNLYEVKSKKHPQIIKKG
Campylobacter jejuni: cjCas9
(SEQ ID NO: 36)
MARILAFDIGISSIGWAFSENDELKDCGVRIFTKVENPKTGESLALP
RRLARSARKRLARRKARLNHLKHLIANEFKLNYEDYQSFDESLAKAY
KGSLISPYELRFRALNELLSKQDFARVILHIAKRRGYDDIKNSDDKE
KGAILKAIKQNEEKLANYQSVGEYLYKEYFQKFKENSKEFTNVRNKK
ESYERCIAQSFLKDELKLIFKKQREFGFSFSKKFEEEVLSVAFYKRA
LKDFSHLVGNCSFFTDEKRAPKNSPLAFMFVALTRIINLLNNLKNTE
GILYTKDDLNALLNEVLKNGTLTYKQTKKLLGLSDDYEFKGEKGTYF
IEFKKYKEFIKALGEHNLSQDDLNEIAKDITLIKDEIKLKKALAKYD
LNQNQIDSLSKLEFKDHLNISFKALKLVTPLMLEGKKYDEACNELNL
KVAINEDKKDFLPAFNETYYKDEVTNPVVLRAIKEYRKVLNALLKKY
GKVHKINIELAREVGKNHSQRAKIEKEQNENYKAKKDAELECEKLGL
KINSKNILKLRLFKEQKEFCAYSGEKIKISDLQDEKMLEIDHIYPYS
RSFDDSYMNKVLVFTKQNQEKLNQTPFEAFGNDSAKWQKIEVLAKNL
PTKKQKRILDKNYKDKEQKNFKDRNLNDTRYIARLVLNYTKDYLDFL
PLSDDENTKLNDTQKGSKVHVEAKSGMLTSALRHTWGFSAKDRNNHL
HHAIDAVIIAYANNSIVKAFSDFKKEQESNSAELYAKKISELDYKNK
RKFFEPFSGFRQKVLDKIDEIFVSKPERKKPSGALHEETFRKEEEFY
QSYGGKEGVLKALELGKIRKVNGKIVKNGDMFRVDIFKHKKTNKFYA
VPIYTMDFALKVLPNKAVARSKKGEIKDWILMDENYEFCFSLYKDSL
ILIQTKDMQEPEFVYYNAFTSSTVSLIVSKHDNKFETLSKNQKILFK
NANEKEVIAKSIGIQNLKVFEKYIVSALGEVTKAEFRQREDFKK
Neisseria meningitidis: nmeCas9
(SEQ ID NO: 37)
MAAFKPNSINYILGLDIGIASVGWAMVEIDEEENPIRLIDLGVRVFE
RAEVPKTGDSLAMARRLARSVRRLTRRRAHRLLRTRRLLKREGVLQA
ANFDENGLIKSLPNTPWQLRAAALDRKLTPLEWSAVLLHLIKHRGYL
SQRKNEGETADKELGALLKGVAGNAHALQTGDFRTPAELALNKFEKE
SGHIRNQRSDYSHTFSRKDLQAELILLFEKQKEFGNPHVSGGLKEGI
ETLLMTQRPALSGDAVQKMLGHCTFEPAEPKAAKNTYTAERFIWLTK
LNNLRILEQGSERPLTDTERATLMDEPYRKSKLTYAQARKLLGLEDT
AFFKGLRYGKDNAEASTLMEMKAYHAISRALEKEGLKDKKSPLNLSP
ELQDEIGTAFSLFKTDEDITGRLKDRIQPEILEALLKHISFDKFVQI
SLKALRRIVPLMEQGKRYDEACAEIYGDHYGKKNTEEKIYLPPIPAD
EIRNPWLRALSQARKVINGVVRRYGSPARIHIETAREVGKSFKDRKE
IEKRQEENRKDREKAAAKFREYFPNFVGEPKSKDILKLRLYEQQHGK
CLYSGKEINLGRLNEKGYVEIDHALPFSRTWDDSFNNKVLVLGSENQ
NKGNQTPYEYFNGKDNSREWQEFKARVETSRFPRSKKQRILLQKFDE
DGFKERNLNDTRYVNRFLCQFVADRMRLTGKGKKRVFASNGQITNLL
RGFWGLRKVRAENDRHHALDAVWACSTVAMQQKITRFVRYKEMNAFD
GKTIDKETGEVLHQKTHFPQPWEFFAQEVMIRVFGKPDGKPEFEEAD
TLEKLRTLLAEKLSSRPEAVHEYVTPLFVSRAPNRKMSGQGHMETVK
SAKRLDEGVSVLRVPLTQLKLKDLEKMVNREREPKLYEALKARLEAH
KDDPAKAFAEPFYKYDKAGNRTQQVKAVRVEQVQKTGVWWRNHNGIA
DNATMVRVDVFEKGDKYYLVPIYSWQVAKGILPDRAWQGKDEEDWQL
IDDSFNFKFSLHPNDLVEVITKKARMFGYFASCHRGTGNINIRIHDL
DHKIGKNGILEGIGVKTALSFQKYQIDELGKEIRPCRLKKRPPVR
Acidaminococcus sp.: asCas12a
(SEQ ID NO: 38)
MTQFEGFTNLYQVSKTLRFELIPQGKTLKHIQEQGFIEEDKARNDHY
KELKPIIDRIYKTYADQCLQLVQLDWENLSAAIDSYRKEKTEETRNA
LIEEQATYRNAIHDYFIGRTDNLTDAINKRHAEIYKGLFKAELFNGK
VLKQLGTVTTTEHENALLRSFDKFTTYFSGFYENRKNVFSAEDISTA
IPHRIVQDNFPKFKENCHIFTRLITAVPSLREHFENVKKAIGIFVST
SIEEVFSFPFYNQLLTQTQIDLYNQLLGGISREAGTEKIKGLNEVLN
LAIQKNDETAHIIASLPHRFIPLFKQILSDRNTLSFILEEFKSDEEV
IQSFCKYKTLLRNENVLETAEALFNELNSIDLTHIFISHKKLETISS
ALCDHWDTLRNALYERRISELTGKITKSAKEKVQRSLKHEDINLQEI
ISAAGKELSEAFKQKTSEILSHAHAALDQPLPTTLKKQEEKEILKSQ
LDSLLGLYHLLDWFAVDESNEVDPEFSARLTGIKLEMEPSLSFYNKA
RNYATKKPYSVEKFKLNFQMPTLASGWDVNKEKNNGAILFVKNGLYY
LGIMPKQKGRYKALSFEPTEKTSEGFDKMYYDYFPDAAKMIPKCSTQ
LKAVTAHFQTHTTPILLSNNFIEPLEITKEIYDLNNPEKEPKKFQTA
YAKKTGDQKGYREALCKWIDFTRDFLSKYTKTTSIDLSSLRPSSQYK
DLGEYYAELNPLLYHISFQRIAEKEIMDAVETGKLYLFQIYNKDFAK
GHHGKPNLHTLYWTGLFSPENLAKTSIKLNGQAELFYRPKSRMKRMA
HRLGEKMLNKKLKDQKTPIPDTLYQELYDYVNHRLSHDLSDEARALL
PNVITKEVSHEIIKDRRFTSDKFFFHVPITLNYQAANSPSKFNQRVN
AYLKEHPETPIIGIDRGERNLIYITVIDSTGKILEQRSLNTIQQFDY
QKKLDNREKERVAARQAWSVVGTIKDLKQGYLSQVIHEIVDLMIHYQ
AVWLENLNFGFKSKRTGIAEKAVYQQFEKMLIDKLNCLVLKDYPAEK
VGGVLNPYQLTDQFTSFAKMGTQSGFLFYVPAPYTSKIDPLTGFVDP
FVWKTIKNHESRKHFLEGFDFLHYDVKTGDFILHFKMNRNLSFQRGL
PGFMPAWDIVFEKNETQFDAKGTPFIAGKRIVPVIENHRFTGRYRDL
YPANELIALLEEKGIVFRDGSNILPKLLENDDSHAIDTMVALIRSVL
QMRNSNAATGEDYINSPVRDLNGVCFDSRFQNPEWPMDADANGAYHI
ALKGQLLLNHLKESKDLKLQNGISNQDWLAYIQELRN
Lachnospiraceae bacterium: IbCas12a:
(SEQ ID NO: 39)
MSKLEKFTNCYSLSKTLRFKAIPVGKTQENIDNKRLLVEDEKRAEDY
KGVKKLLDRYYLSFINDVLHSIKLKNLNNYISLFRKKTRTEKENKEL
ENLEINLRKEIAKAFKGNEGYKSLFKKDIIETILPEFLDDKDEIALV
NSFNGFTTAFTGFFDNRENMFSEEAKSTSIAFRCINENLTRYISNMD
IFEKVDAIFDKHEVQEIKEKILNSDYDVEDFFEGEFFNFVLTQEGID
VYNAIIGGFVTESGEKIKGLNEYINLYNQKTKQKLPKFKPLYKQVLS
DRESLSFYGEGYTSDEEVLEVFRNTLNKNSEIFSSIKKLEKLFKNFD
EYSSAGIFVKNGPAISTISKDIFGEWNVIRDKWNAEYDDIHLKKKAW
TEKYEDDRRKSFKKIGSFSLEQLQEYADADLSWEKLKEIIIQKVDEI
YKVYGSSEKLFDADFVLEKSLKKNDAVVAIMKDLLDSVKSFENYIKA
FFGEGKETNRDESFYGDFVLAYDILLKVDHIYDAIRNYVTQKPYSKD
KFKLYFQNPQFMGGWDKDKETDYRATILRYGSKYYLAIMDKKYAKCL
QKIDKDDVNGNYEKINYKLLPGPNKMLPKVFFSKKWMAYYNPSEDIQ
KIYKNGTFKKGDMFNLNDCHKLIDFFKDSISRYPKWSNAYDFNFSET
EKYKDIAGFYREVEEQGYKVSFESASKKEVDKLVEEGKLYMFQIYNK
DFSDKSHGTPNLHTMYFKLLFDENNHGQIRLSGGAELFMRRASLKKE
ELVVHPANSPIANKNPDNPKKTTTLSYDVYKDKRFSEDQYELHIPIA
INKCPKNIFKINTEVRVLLKHDDNPYVIGIDRGERNLLYIVWVDGKG
NIVEQYSLNEIINNFNGIRIKTDYHSLLDKKEKERFEARQNWTSIEN
IKELKAGYISQVVHKICELVEKYDAVIALEDLNSGFKNSRVKVEKQV
YQKFEKMLIDKLNYMVDKKSNPCATGGALKGYQITNKFESFKSMSTQ
NGFIFYIPAWLTSKIDPSTGFVNLLKTKYTSIADSKKFISSFDRIMY
VPEEDLFEFALDYKNFSRTDADYIKKWKLYSYGNRIRIFRNPKKNNV
FDWEEVCLTSAYKELFNKYGINYQQGDIRALLCEQSDKAFYSSFMAL
MSLMLQMRNSITGRTDVDFLISPVKNSDGIFYDSRNYEAQENAILPK
NADANGAYNIARKVLWAIGQFKKAEDEKLDKVKIAISNKEWLEYAQT
SVKH
Leptotrichia shahii: LshCas13a
(SEQ ID NO: 40)
MGNLFGHKRWYEVRDKKDFKIKRKVKVKRNYDGNKYILNINENNNKE
KIDNNKFIRKYINYKKNDNILKEFTRKFHAGNILFKLKGKEGIIRIE
NNDDFLETEEWLYIEAYGKSEKLKALGITKKKIIDEAIRQGITKDDK
KIEIKRQENEEEIEIDIRDEYTNKTLNDCSIILRIIENDELETKKSI
YEIFKNINMSLYKIIEKIIENETEKVFENRYYEEHLREKLLKDDKID
VILTNFMEIREKIKSNLEILGFVKFYLNVGGDKKKSKNKKMLVEKIL
NINVDLTVEDIADFVIKELEFWNITKRIEKVKKVNNEFLEKRRNRTY
IKSYVLLDKHEKFKIERENKKDKIVKFFVENIKNNSIKEKIEKILAE
FKIDELIKKLEKELKKGNCDTEIFGIFKKHYKVNFDSKKFSKKSDEE
KELYKIIYRYLKGRIEKILVNEQKVRLKKMEKIEIEKILNESILSEK
ILKRVKQYTLEHIMYLGKLRHNDIDMTTVNTDDFSRLHAKEELDLEL
ITFFASTNMELNKIFSRENINNDENIDFFGGDREKNYVLDKKILNSK
IKIIRDLDFIDNKNNITNNFIRKFTKIGTNERNRILHAISKERDLQG
TQDDYNKVINIIQNLKISDEEVSKALNLDVVFKDKKNIITKINDIKI
SEENNNDIKYLPSFSKVLPEILNLYRNNPKNEPFDTIETEKIVLNAL
IYVNKELYKKLILEDDLEENESKNIFLQELKKTLGNIDEIDENIIEN
YYKNAQISASKGNNKAIKKYQKKVIECYIGYLRKNYEELFDFSDFKM
NIQEIKKQIKDINDNKTYERITVKTSDKTIVINDDFEYIISIFALLN
SNAVINKIRNRFFATSVWLNTSEYQNIIDILDEIMQLNTLRNECITE
NWNLNLEEFIQKMKEIEKDFDDFKIQTKKEIFNNYYEDIKNNILTEF
KDDINGCDVLEKKLEKMFDDETKFEIDKKSNILQDEQRKLSNINKKD
LKKKVDQYIKDKDQEIKSKILCRIIFNSDFLKKYKKEIDNLIEDMES
ENENKFQEIYYPKERKNELYIYKKNLFLNIGNPNFDKIYGLISNDIK
MADAKFLFNIDGKNIRKNKISEIDAILKNLNDKLNGYSKEYKEKYIK
KLKENDDFFAKNIQNKNYKSFEKDYNRVSEYKKIRDLVEFNYLNKIE
SYLIDINWKLAIQMARFERDMHYIVNGLRELGIIKLSGYNTGISRAY
PKRNGSDGFYTTTAYYKFFDEESYKKFEKICYGFGIDLSENSEINKP
ENESIRNYISHFYIVRNPFADYSIAEQIDRVSNLLSYSTRYNNSTYA
SVFEVFKKDVNLDYDELKKKFKLIGNNDILERLMKPKKVSVLELESY
NSDYIKNLIIELLTKIENTNDTL
Leptotrichia wadeii: LwaCas13a
(SEQ ID NO: 41)
MKVTKVDGISHKKYIEEGKLVKSTSEENRTSERLSELLSIRLDIYIK
NPDNASEEENRIRRENLKKFFSNKVLHLKDSVLYLKNRKEKNAVQDK
NYSEEDISEYDLKNKNSFSVLKKILLNEDVNSEELEIFRKDVEAKLN
KINSLKYSFEENKANYQKINENNVEKVGGKSKRNIIYDYYRESAKRN
DYINNVQEAFDKLYKKEDIEKLFFLIENSKKHEKYKIREYYHKIIGR
KNDKENFAKIIYEEIQNVNNIKELIEKIPDMSELKKSQVFYKYYLDK
EELNDKNIKYAFCHFVEIEMSQLLKNYVYKRLSNISNDKIKRIFEYQ
NLKKLIENKLLNKLDTYVRNCGKYNYYLQVGEIATSDFIARNRQNEA
FLRNIIGVSSVAYFSLRNILETENENGITGRMRGKTVKNNKGEEKYV
SGEVDKIYNENKQNEVKENLKMFYSYDFNMDNKNEIEDFFANIDEAI
SSIAHGIVHFNLELEGKDIFAFKNIAPSEISKKMFQNEINEKKLKLK
IFKQLNSANVFNYYEKDVIIKYLKNTKFNFVNKNIPFVPSFTKLYNK
IEDLRNTLKFFWSVPKDKEEKDAQIYLLKNIYYGEFLNKFVKNSKVF
FKITNEVIKINKQRNQKTGHYKYQKFENIEKTVPVEYLAIIQSREMI
NNQDKEEKNTYIDFIQQIFLKGFIDYLNKNNLKYIESNNNNDNNDIF
SKIKIKKDNKEKYDKILKNYEKHNRNKEIPHEINEFVREIKLGKILK
YTENLNMFYLILKLLNHKELTNLKGSLEKYQSANKEETFSDELELIN
LLNLDNNRVTEDFELEANEIGKFLDFNENKIKDRKELKKFDTNKIYF
DGENIIKHRAFYNIKKYGMLNLLEKIADKAKYKISLKELKEYSNKKN
EIEKNYTMQQNLHRKYARPKKDEKFNDEDYKEYEKAIGNIQKYTHLK
NKVEFNELNLLQGLLLKILHRLVGYTSIWERDLRFRLKGEFPENHYI
EEIFNFDNSKNVKYKSGQIVEKYINFYKELYKDNVEKRSIYSDKKVK
KLKQEKKDLYIANYIAHFNYIPHAEISLLEVLENLRKLLSYDRKLKN
AIMKSIVDILKEYGFVATFKIGADKKIEIQTLESEKIVHLKNLKKKK
LMTDRNSEELCELVKVMFEYKALE
Pleckstrin homology domain of Homo sapiens
phospholipase C51 (hPLC51)
(SEQ ID NO: 42)
MDSGRDFLTLHGLQDDEDLQALLKGSQLLKVKSSSWRRERFYKLQED
CKTIWQESRKVMRTPESQLFSIEDIQEVRMGHRTEGLEKFARDVPED
RCFSIVFKDQRNTLDLIAPSPADAQHWVLGLHKIIHHSGSMDQRQKL
QHWIHSCLRKADKNKDNKMSFKELQNFLKELNIQ
Pleckstrin homology domain of Homo sapiens
Akt1 (hAkt)
(SEQ ID NO: 43)
MSDVAIVKEGWLHKRGEYIKTWRPRYFLLKNDGTFIGYKERPQDVDQ
REAPLNNFSVAQCQLMKTERPRPNTFIIRCLQWTTVIERTFHVETPE
EREEWTTAIQTVADGLKKQEEEEMDFRSGSPSDNSGAEEMEVSLAKP
KHRVTMNEFEYLKLLGKGTFGKVDPPV
Pleckstrin homology domain of Homo sapiens
PDPK1 (hPDPKI)
(SEQ ID NO: 44)
KMGPVDKRKGLFARRRQLLLTEGPHLYYVDPVNKVLKGEIPWSQELR
PEAKNFKTFFVHTPNRTYYLMDPSGNAHKWCRKIQEVWRQRYQSH
Herpes simplex virus (HSV) type 1: VP16
Transcription Activation Domain
(SEQ ID NO: 45)
PTDALDDFDLDMLPADALDDFDLDMLPADALDDFDLDM
Herpes simplex virus (HSV) type 1 &
Synthetic: VP64
(SEQ ID NO: 46)
GRADALDDFDLDMLGSDALDDFDLDMLGSDALDDFDLDMLGSDALDD
FDLDML
Homo sapiens: P65
(SEQ ID NO: 47)
SQYLPDTDDRHRIEEKRKRTYETFKSIMKKSPFSGPTDPRPPPRRIA
VPSRSSASVPKPAPQPYPFTSSLSTINYDEFPTMVFPSGQISQASAL
APAPPQVLPQAPAPAPAPAMVSALAQAPAPVPVLAPGPPQAVAPPAP
KPTQAGEGTLSEALLQLQFDDEDLGALLGNSTDPAVFTDLASVDNSE
FQQLLNQGIPVAPHTTEPMLMEYPEAITRLVTGAQRPPDPAPAPLGA
PGLPNGLLSGDEDFSSIADMDFSALL
Kaposi's Sarcoma-Associated Herpesvirus
Transactivator: RTA
(SEQ ID NO: 48)
RDSREGMFLPKPEAGSAISDVFEGREVCQPKRIRPFHPPGSPWANRP
LPASLAPTPTGPVHEPVGSLTPAPVPQPLDPAPAVTPEASHLLEDPD
EETSQAVKALREMADIVIPQKEEAAICGQMDLSHPPPRGHLDELIII
LESMIEDLNLDSPLIPELNEILDTFLNDECLLHAMHISTGLSIFDTS
LF
Homo sapiens: KRAB
(SEQ ID NO: 49)
MDAKSLTAWSRTLVTFKDVFVDFTREEWKLLDTAQQIVYRNVMLENY
KNLVSLGYQLTKPDVILRLEKGEEP
Homo sapiens. MeCP2
(SEQ ID NO: 50)
EASVQVKRVLEKSPGKLLVKMPFQASPGGKGEGGGATTSAQVMVIKR
PGRKRKAEADPQAIPKKRGRKPGSVVAAAAAEAKKKAVKESSIRSVQ
ETVLPIKKRKTRETVSIEVKEWKPLLVSTLGEKSGKGLKTCKSPGRK
SKESSPKGRSSSASSPPKKEHHHHHHHAESPKAPMPLLPPPPPPEPQ
SSEDPISPPEPQDLSSSICKEEKMPRAGSLESDGCPKEPAKIQPMVA
AAATTTTTTTTTVAEKYKHRGEGERKDIVSSSMPRPNREEPVDSRTP
VTERVS
Homo sapiens: Tet1
(SEQ ID NO: 51)
LPTCSCLDRVIQKDKGPYYTHLGAGPSVAAVREIMENRYGQKGNAIR
IEIWYTGKEGKSSHGCPIAKWWLRRSSDEEKVLCLVRQRTGHHCPTA
VMVVLIMVWDGIPLPMADRLYTELTENLKSYNGHPTDRRCTLNENRT
CTCQGIDPETCGASFSFGCSWSMYFNGCKFGRSPSPRRFRIDPSSPL
HEKNLEDNLQSLATRLAPIYKQYAPVAYQNQVEYENVARECRLGSKE
GRPFSGVTACLDFCAHPHRDIHNMNNGSTVVCTLTREDNRSLGVIPQ
DEQLHVLPLYKLSDTDEFGSKEGMEAKIKSGAIEVLAPRRKKRTCFT
QPVPRSGKKRAAMMTEVLAHKIRAVEKKPIPRIKRKNNSTTTNNSKP
SSLPTLGSNTETVQPEVKSETEPHFILKSSDNTKTYSLMPSAPHPVK
EASPGFSWSPKTASATPAPLKNDATASCGFSERSSTPHCTMPSGRLS
GANAAAADGPGISQLGEVAPLPTLSAPVMEPLINSEPSTGVTEPLTP
HQPNHQPSFLTSPQDLASSPMEEDEQHSEADEPPSDEPLSDDPLSPA
EEKLPHIDEYWSDSEHIFLDANIGGVAIAPAHGSVLIECARRELHAT
TPVEHPNRNHPTRLSLVFYQHKNLNKPQHGFELNKIKFEAKEAKNKK
MKASEQKDQAANEGPEQSSEVNELNQIPSHKALTLTHDNVVTVSPYA
LTHVAGPYNHWW
Homo sapiens: Dnmt3a
(SEQ ID NO: 52)
MPAMPSSGPGDTSSSAAEREEDRKDGEEQEEPRGKEERQEPSTTARK
VGRPGRKRKHPPVESGDTPKDPAVISKSPSMAQDSGASELLPNGDLE
KRSEPQPEEGSPAGGQKGGAPAEGEGAAETLPEASRAVENGCCTPKE
GRGAPAEAGKEQKETNIESMKMEGSRGRLRGGLGWESSLRQRPMPRL
TFQAGDPYYISKRKRDEWLARWKREAEKKAKVIAGMNAVEENQGPGE
SQKVEEASPPAVQQPTDPASPTVATTPEPVGSDAGDKNATKAGDDEP
EYEDGRGFGIGELVWGKLRGFSWWPGRIVSWWMTGRSRAAEGTRWWM
WFGDGKFSVVCVEKLMPLSSFCSAFHQATYNKQPMYRKAIYEVLQVA
SSRAGKLFPVCHDSDESDTAKAVEVQNKPMIEWALGGFQPSGPKGLE
PPEEEKNPYKEVYTDMWVEPEAAAYAPPPPAKKPRKSTAEKPKVKEI
IDERTRERLVYEVRQKCRNIEDICISCGSLNVTLEHPLFVGGMCQNC
KNCFLECAYQYDDDGYQSYCTICCGGREVLMCGNNNCCRCFCVECVD
LLVGPGAAQAAIKEDPWNCYMCGHKGTYGLLRRREDWPSRLQMFFAN
NHDQEFDPPKVYPPVPAEKRKPIRVLSLFDGIATGLLVLKDLGIQVD
RYIASEVCEDSITVGMVRHQGKIMYVGDVRSVTQKHIQEWGPFDLVI
GGSPCNDLSIVNPARKGLYEGTGRLFFEFYRLLHDARPKEGDDRPFF
WLFENVVAMGVSDKRDISRFLESNPVMIDAKEVSAAHRARYFWGNLP
GMNRPLASTVNDKLELQECLEHGRIAKFSKVRTITTRSNSIKQGKDQ
HFPVFMNEKEDILWCTEMERVFGFPVHYTDVSNMSRLARQRLLGRSW
SVPVIRHLFAPLKEYFACV
Indiana vesiculovirus, formerly Vesicular
stomatitis Indiana virus G Protein: VSVG
(SEQ ID NO: 53)
MKCLLYLAFLFIGVNCKFTIVFPHNQKGNWKNVPSNYHYCPSSSDLN
WHNDLIGTALQVKMPKSHKAIQADGWMCHASKWVTTCDFRWYGPKYI
THSIRSFTPSVEQCKESIEQTKQGTWLNPGFPPQSCGYATVTDAEAV
IVQVTPHHVLVDEYTGEWVDSQFINGKCSNYICPTVHNSTTWHSDYK
VKGLCDSNLISMDITFFSEDGELSSLGKEGTGFRSNYFAYETGGKAC
KMQYCKHWGVRLPSGVWFEMADKDLFAAARFPECPEGSSISAPSQTS
VDVSLIQDVERILDYSLCQETWSKIRAGLPISPVDLSYLAPKNPGTG
PAFTIINGTLKYFETRYIRVDIAAPILSRMVGMISGTTTERELWDDW
APYEDVEIGPNGVLRTSSGYKFPLYMIGHGMLDSDLHLSSKAQVFEH
PHIQDAASQLPDDESLFFGDTGLSKNPIELVEGWFSSWKSSIASFFF
IIGLIIGLFLVLRVGIHLCIKLKHTKKRQIYTDIEMNRLGK
Baculovirus envelope glycoprotein GP64
(SEQ ID NO: 54)
MVSAIVLYVLLAAAAHSAFAAEHCNAQMKTGPYKIKNLDITPPKETL
QKDVEITIVETDYNENVIIGYKGYYQAYAYNGGSLDPNTRVEETMKT
LNVGKEDLLMWSIRQQCEVGEELIDRWGSDSDDCFRDNEGRGQWVKG
KELVKRQNNNHFAHHTCNKSWRCGISTSKMYSRLECQDDTDECQVYI
LDAEGNPINVTVDTVLHRDGVSMILKQKSTFTTRQIKAACLLIKDDK
NNPESVTREHCLIDNDIYDLSKNTWNCKFNRCIKRKVEHRVKKRPPT
WRHNVRAKYTEGDTATKGDLMHIQEELMYENDLLKMNIELMHAHINK
LNNMLHDLIVSVAKVDERLIGNLMNNSVSSTFLSDDTFLLMPCTNPP
AHTSNCYNNSIYKEGRWVANTDSSQCIDFSNYKELAIDDDVEFWIPT
IGNTTYHDSWKDASGWSFIAQQKSNLITTMENTKFGGVGTSLSDITS
MAEGELAAKLTSFMFGHVVNFVIILIVILFLYCMIRNRNRQY
Human immunodeficiency virus gp160
(SEQ ID NO: 55)
MRVKEKYQHLWRWGWRWGTMLLGMLMICSATEKLWVTVYYGVPVWKE
ATTTLFCASDAKAYDTEVHNVWATHACVPTDPNPQEVVLVNVTENFN
MWKNDMVEQMHEDIISLWDQSLKPCVKLTPLCVSLKCTDLKNDTNTN
SSSGRMIMEKGEIKNCSFNISTSIRGKVQKEYAFFYKLDIIPIDNDT
TSYKLTSCNTSVITQACPKVSFEPIPIHYCAPAGFAILKCNNKTFNG
TGPCTNVSTVQCTHGIRPVVSTQLLLNGSLAEEEVVIRSVNFTDNAK
TIIVQLNTSVEINCTRPNNNTRKRIRIQRGPGRAFVTIGKIGNMRQA
HCNISRAKWNNTLKQIASKLREQFGNNKTIIFKQSSGGDPEIVTHSF
NCGGEFFYCNSTQLFNSTWFNSTWSTEGSNNTEGSDTITLPCRIKQI
INMWQKVGKAMYAPPISGQIRCSSNITGLLLTRDGGNSNNESEIFRP
GGGDMRDNWRSELYKYKVVKIEPLGVAPTKAKRRVVQREKRAVGIGA
LFLGFLGAAGSTMGAASMTLTVQARQLLSGIVQQQNNLLRAIEAQQH
LLQLTVWGIKQLQARILAVERYLKDQQLLGIWGCSGKLICTTAVPWN
ASWSNKSLEQIWNHTTWMEWDREINNYTSLIHSLIEESQNQQEKNEQ
ELLELDKWASLWNWFNITNWLWYIKLFIMIVGGLVGLRIVFAVLSIV
NRVRQGYSPLSFQTHLPTPRGPDRPEGIEEEGGERDRDRSIRLVNGS
LALIWDDLRSLCLFSYHRLRDLLLIVTRIVELLGRRGWEALKYWWNL
LQYWSQELKNSAVSLLNATAIAVAEGTDRVIEVQGACRAIRHIPRRI
RQGLERILL
Endogenous feline virus RD114 ENV
(SEQ ID NO: 56)
MKLPTGMVILCSLIIVRAGFDDPRKAIALVQKQHGKPCECSGGQVSE
APPNSIQQVTCPGKTAYLMTNQKWKCRVTPKISPSGGELQNCPCNTF
QDSMHSSCYTEYRQCRRINKTYYTATLLKIRSGSLNEVQILQNPNQL
LQSPCRGSINQPVCWSATAPIHISDGGGPLDTKRVWTVQKRLEQIHK
AMTPELQYHPLALPKVRDDLSLDARTFDILNTTFRLLQMSNFSLAQD
CWLCLKLGTPTPLAIPTPSLTYSLADSLANASCQIIPPLLVQPMQFS
NSSCLSSPFINDTEQIDLGAVTFTNCTSVANVSSPLCALNGSVFLCG
NNMAYTYLPQNWTRLCVQASLLPDIDINPGDEPVPIPAIDHYIHRPK
RAVQFIPLLAGLGITAAFTTGATGLGVSVTQYTKLSHQLISDVQVLS
GTIQDLQDQVDSLAEWLQNRRGLDLLTAEQGGICLALQEKCCFYANK
SGIVRNKIRTLQEELQKRRESLATNPLWTGLQGFLPYLLPLLGPLLT
LLLILTIGPCVFSRLMAFINDRLNVVHAMVLAQQYQALKAEEEAQD
Homo sapiens: CD9 Complete Protein
(SEQ ID NO: 57)
MSPVKGGTKCIKYLLFGFNFIFWLAGIAVLAIGLWLRFDSQTKSIFE
QETNNNNSSFYTGVYILIGAGALMMLVGFLGCCGAVQESQCMLGLFF
GFLLVIFAIEIAAAIWGYSHKDEVIKEVQEFYKDTYNKLKTKDEPQR
ETLKAIHYALNCCGLAGGVEQFISDICPKKDVLETFTVKSCPDAIKE
VFDNKFHIIGAVGIGIAVVMIFGMIFSMILCCAIRRNREMV
Homo sapiens: CD63 Complete Protein
(SEQ ID NO: 58)
MAVEGGMKCVKFLLYVLLLAFCACAVGLIAVGVGAQLVLSQTIIQGA
TPGSLLPVVIIAVGVFLFLVAFVGCCGACKENYCLMITFAIFLSLIM
LVEVAAAIAGYVFRDKVMSEFNNNFRQQMENYPKNNHTASILDRMQA
DFKCCGAANYTDWEKIPSMSKNRVPDSCCINVTVGCGINFNEKAIHK
EGCVEKIGGWLRKNVLVVAAAALGIAFVEVLGIVFACCLVKSIRSGY
EVM
Homo sapiens: CD81 Complete Protein
(SEQ ID NO: 59)
MGVEGCTKCIKYLLFVFNFVFWLAGGVILGVALWLRHDPQTTNLLYL
ELGDKPAPNTFYVGIYILIAVGAVMMFVGFLGCYGAIQESQCLLGTF
FTCLVILFACEVAAGIWGFVNKDQIAKDVKQFYDQALQQAWDDDANN
AKAVVKTFHETLDCCGSSTLTALTTSVLKNNLCPSGSNIISNLFKED
CHQKIDDLFSGKLYLIGIAAIWAVIMIFEMILSMVLCCGIRNSSVY
Homo sapiens: CD47 “Self Hairpin” 10
Amino Acids
(SEQ ID NO: 60)
EVTELTREGE
Homo sapiens: CD47 “Self Hairpin” 21
Amino Acids
(SEQ ID NO: 61)
GNYTCEVTELTREGETIIELK
Homo sapiens: CD47 Complete Protein
(SEQ ID NO: 62)
MWPLVAALLLGSACCGSAQLLFNKTKSVEFTFCNDTVVIPCFVTNME
AQNTTEVYVKWKFKGRDIYTFDGALNKSTVPTDFSSAKIEVSQLLKG
DASLKMDKSDAVSHTGNYTCEVTELTREGETIIELKYRVVSWFSPNE
NILIVIFPIFAILLFWGQFGIKTLKYRSGGMDEKTIALLVAGLVITV
IVIVGAILFVPGEYSLKNATGLGLIVTSTGILILLHYYVFSTAIGLT
SFVIAILVIQVIAYILAVGLSLCIAACIPMHGPLLISGLSILALAQL
LGLVYMKFVE
AAV2: REP52
(SEQ ID NO: 63)
MELVGWLVDKGITSEKQWIQEDQASYISFNAASNSRSQIKAALDNAG
KIMSLTKTAPDYLVGQQPVEDISSNRIYKILELNGYDPQYAASVFLG
WATKKFGKRNTIWLFGPATTGKTNIAEAIAHTVPFYGCVNWTNENFP
FNDCVDKMVIWWEEGKMTAKWVESAKAILGGSKVRVDQKCKSSAQID
PTPVIVTSNTNMCAVIDGNSTTFEHQQPLQDRMFKFELTRRLDHDFG
KVTKQEVKDFFRWAKDHWEVEHEFYVKKGGAKKRPAPSDADISEPKR
VRESVAQPSTSDAEASINYADRYQNKCSRHVGMNLMLFPCRQCERMN
QNSNICFTHGQKDCLECFPVSESQPVSWKKAYQKLCYIHHIMGKVPD
ACTACDLVNVDLDDCIFEQ
AAV2: REP78
(SEQ ID NO: 64)
MPGFYEIVIKVPSDLDEHLPGISDSFVNWAEKEWELPPDSDMDLNLI
EQAPLTVAEKLQRDFLTEWRRVSKAPEALFFVQFEKGESYFHMHVLV
ETTGVKSMVLGRFLSQIREKLIQRIYRGIEPTLPNWFAVTKTRNGAG
GGNKWDECYIPNYLLPKTQPELQWAWTNMEQYLSACLNLTERKRLVA
QHLTHVSQTQEQNKENQNPNSDAPVIRSKTSARYMELVGWLVDKGIT
SEKQWIQEDQASYISFNAASNSRSQIKAALDNAGKIMSLTKTAPDYL
VGQQPVEDISSNRIYKILELNGYDPQYAASVFLGWATKKFGKRNTIW
LFGPATTGKTNIAEAIAHTVPFYGCVNWTNENFPFNDCVDKMVIWWE
EGKMTAKVVESAKAILGGSKVRVDQKCKSSAQIDPTPVIVTSNTNMC
AVIDGNSTTFEHQQPLQDRMFKFELTRRLDHDFGKVTKQEVKDFFRW
AKDHWEVEHEFYVKKGGAKKRPAPSDADISEPKRVRESVAQPSTSDA
EASINYADRYQNKCSRHVGMNLMLFPCRQCERMNQNSNICFTHGQKD
CLECFPVSESQPVSVVKKAYQKLCYIHHIMGKVPDACTACDLVNVDL
DDCIFEQ
AAV2: VP1
(SEQ ID NO: 65)
MAADGYLPDWLEDTLSEGIRQWWKLKPGPPPPKPAERHKDDSRGLVL
PGYKYLGPFNGLDKGEPVNEADAAALEHDKAYDRQLDSGDNPYLKYN
HADAEFQERLKEDTSFGGNLGRAVFQAKKRVLEPLGLVEEPVKTAPG
KKRPVEHSPVEPDSSSGTGKAGQQPARKRLNFGQTGDADSVPDPQPL
GQPPAAPSGLGTNTMATGSGAPMADNNEGADGVGNSSGNWHCDSTWM
GDRVITTSTRTWALPTYNNHLYKQISSQSGASNDNHYFGYSTPWGYF
DFNRFHCHFSPRDWQRLINNNWGFRPKRLNFKLFNIQVKEVTQNDGT
TTIANNLTSTVQVFTDSEYQLPYVLGSAHQGCLPPFPADVFMVPQYG
YLTLNNGSQAVGRSSFYCLEYFPSQMLRTGNNFTFSYTFEDVPFHSS
YAHSQSLDRLMNPLIDQYLYYLSRTNTPSGTTTQSRLQFSQAGASDI
RDQSRNWLPGPCYRQQRVSKTSADNNNSEYSWTGATKYHLNGRDSLV
NPGPAMASHKDDEEKFFPQSGVLIFGKQGSEKTNVDIEKVMITDEEE
IRTTNPVATEQYGSVSTNLQRGNRQAATADVNTQGVLPGMVWQDRDV
YLQGPIWAKIPHTDGHFHPSPLMGGFGLKHPPPQILIKNTPVPANPS
TTFSAAKFASFITQYSTGQVSVEIEWELQKENSKRWNPEIQYTSNYN
KSVNVDFTVDTNGVYSEPRPIGTRYLTRNL
AAV2: VP2
(SEQ ID NO: 66)
APGKKRPVEHSPVEPDSSSGTGKAGQQPARKRLNFGQTGDADSVPDP
QPLGQPPAAPSGLGTNTMATGSGAPMADNNEGADGVGNSSGNWHCDS
TWMGDRVITTSTRTWALPTYNNHLYKQISSQSGASNDNHYFGYSTPW
GYFDFNRFHCHFSPRDWQRLINNNWGFRPKRLNFKLFNIQVKEVTQN
DGTTTIANNLTSTVQVFTDSEYQLPYVLGSAHQGCLPPFPADVFMVP
QYGYLTLNNGSQAVGRSSFYCLEYFPSQMLRTGNNFTFSYTFEDVPF
HSSYAHSQSLDRLMNPLIDQYLYYLSRTNTPSGTTTQSRLQFSQAGA
SDIRDQSRNWLPGPCYRQQRVSKTSADNNNSEYSWTGATKYHLNGRD
SLVNPGPAMASHKDDEEKFFPQSGVLIFGKQGSEKTNVDIEKVMITD
EEEIRTTNPVATEQYGSVSTNLQRGNRQAATADVNTQGVLPGMVWQD
RDVYLQGPIWAKIPHTDGHFHPSPLMGGFGLKHPPPQILIKNTPVPA
NPSTTFSAAKFASFITQYSTGQVSVEIEWELQKENSKRWNPEIQYTS
NYNKSVNVDFTVDTNGVYSEPRPIGTRYLTRNL
AAV2: VP3
(SEQ ID NO: 67)
MATGSGAPMADNNEGADGVGNSSGNWHCDSTWMGDRVITTSTRTWAL
PTYNNHLYKQISSQSGASNDNHYFGYSTPWGYFDFNRFHCHFSPRDW
QRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRS
SFYCLEYFPSQMLRTGNNFTFSYTFEDVPFHSSYAHSQSLDRLMNPL
IDQYLYYLSRTNTPSGTTTQSRLQFSQAGASDIRDQSRNWLPGPCYR
QQRVSKTSADNNNSEYSWTGATKYHLNGRDSLVNPGPAMASHKDDEE
KFFPQSGVLIFGKQGSEKTNVDIEKVMITDEEEIRTTNPVATEQYGS
VSTNLQRGNRQAATADVNTQGVLPGMVWQDRDVYLQGPIWAKIPHTD
GHFHPSPLMGGFGLKHPPPQILIKNTPVPANPSTTFSAAKFASFITQ
YSTGQVSVEIEWELQKENSKRWNPEIQYTSNYNKSVNVDFTVDTNGV
YSEPRPIGTRYLTRNL
Synthetic: Myc-Tagged Anti CD19 scFv
(SEQ ID NO: 68)
EQKLISEEDLDIQMTQTTSSLSASLGDRVTISCRASQDISKYLNWYQ
QKPDGTVKLLIYHTSRLHSGVPSRFSGSGSGTDYSLTISNLEQEDIA
TYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGG
SYAMDYWGQGTSVTVSS
Synthetic: dDZF1
(SEQ ID NO: 69)
FKCEHCRILFLDHVMFTIHMGCHGFRDPFKCNMCGEKCDGPVGLFVH
MARNAHGEKPFYCEHCEITFRDWMYSLHKGYHGFRDPFECNICGYHS
QDRYEFSSHIVRGEH
Synthetic: dDZF2
(SEQ ID NO: 70)
HHCQHCDMYFADNILYTIHMGCHSCDDVFKCNMCGEKCDGPVGLFVH
MARNAHGEKPTKCVHCGIVFLDEVMYALHMSCHGFRDPFECNICGYH
SQDRYEFSSHIVRGEH
Synthetic: DmrA
(SEQ ID NO: 71)
MGRGVQVETISPGDGRTFPKRGQTCVHYTGMLEDGKKFDSSRDRNKP
FKFMLGKQEVIRGWEEGVAQMSVGQRAKLTISPDYAYGATGHPGIIP
PHATLVFDVELLKLE
Synthetic: DmrB
(SEQ ID NO: 72)
MASRGVQVETISPGDGRTFPKRGQTCWHYTGMLEDGKKVDSSRDRNK
PFKFMLGKQEVIRGWEEGVAQMSVGQRAKLTISPDYAYGATGHPGII
PPHATLVFDVELLKLE
Synthetic: DmrC
(SEQ ID NO: 73)
MGSRILWHEMWHEGLEEASRLYFGERNVKGMFEVLEPLHAMMERGPQ
TLKETSFNQAYGRDLMEAQEWCRKYMKSGNVKDLLQAWDLYYHVFRR
ISK
Homo sapiens/Synthetic: FKBP
(SEQ ID NO: 74)
MGVQVETISPGDGRTFPKRGQTCWHYTGMLEDGKKFDSSRDRNKPFK
FMLGKQEVIRGWEEGVAQMSVGQRAKLTISPDYAYGATGHPGIIPPH
ATLVFDVELLKLE
Homo sapiens/Synthetic: FRB
(SEQ ID NO: 75)
QGMLEMWHEGLEEASRLYFGERNVKGMFEVLEPLHAMMERGPQTLKE
TSFNQAYGRDLMEAQEWCRKYMKSGNVKDLLQAWDLYYHVFRRISK+0
Synthetic: Anti-GCN4 scFv
(SEQ ID NO: 76)
MGPDIVMTQSPSSLSASVGDRVTITCRSSTGAVTTSNYASWVQEKPG
KLFKGLIGGTNNRAPGVPSRFSGSLIGDKATLTISSLQPEDFATYFC
ALWYSNHWVFGQGTKVELKRGGGGSGGGGSGGGGSSGGGSEVKLLES
GGGLVQPGGSLKLSCAVSGFSLTDYGVNVWRQAPGRGLEWIGVIWGD
GITDYNSALKDRFIISKDNGKNTVYLQMSKVRSDDTALYYCVTGLFD
YWGQGTLVTVSSYPYDVPDYAGGGGGSGGGGSGGGGSGGGGS
Synthetic: 10x-GCN4 Repeats
(SEQ ID NO: 77)
EELLSKNYHLENEVARLKKGSGSGEELLSKNYHLENEVARLKKGSGS
GEELLSKNYHLENEVARLKKGSGSGEELLSKNYHLENEVARLKKGSG
SGEELLSKNYHLENEVARLKKGSGSGEELLSKNYHLENEVARLKKGS
GSGEELLSKNYHLENEVARLKKGSGSGEELLSKNYHLENEVARLKKG
SGSGEELLSKNYHLENEVARLKKGSGSGEELLSKNYHLENEVARLKK
GS
Synthetic: 24x-GCN4 Repeats
(SEQ ID NO: 78)
EELLSKNYHLENEVARLKKGSGSGEELLSKNYHLENEVARLKKGSGS
GEELLSKNYHLENEVARLKKGSGSGEELLSKNYHLENEVARLKKGSG
SGEELLSKNYHLENEVARLKKGSGSGEELLSKNYHLENEVARLKKGS
GSGEELLSKNYHLENEVARLKKGSGSGEELLSKNYHLENEVARLKKG
SGSGEELLSKNYHLENEVARLKKGSGSGEELLSKNYHLENEVARLKK
GSGSGEELLSKNYHLENEVARLKKGSGSGEELLSKNYHLENEVARLK
KGSGSGEELLSKNYHLENEVARLKKGSGSGEELLSKNYHLENEVARL
KKGSGSGEELLSKNYHLENEVARLKKGSGSGEELLSKNYHLENEVAR
LKKGSGSGEELLSKNYHLENEVARLKKGSGSGEELLSKNYHLENEVA
RLKKGSGSGEELLSKNYHLENEVARLKKGSGSGEELLSKNYHLENEV
ARLKKGSGSGEELLSKNYHLENEVARLKKGSGSGEELLSKNYHLENE
VARLKKGSGSGEELLSKDYHLENEVARLKKGSGSGEELLSKNYHLEN
EVARLKKGS
Synthetic: GFP-targeting Nanobody
(SEQ ID NO: 79)
VQLVESGGALVQPGGSLRLSCAASGFPVNRYSMRWYRQAPGKEREVW
AGMSSAGDRSSYEDSVKGRFTISRDDARNTVYLQMNSLKPEDTAVYY
SNVNVGFEYWGQGTQVTVSS
Nostoc punctiforme: Npu DnaE N-terminal
Split Intein
(SEQ ID NO: 80)
CLSYETEILTVEYGLLPIGKIVEKRIECTVYSVDNNGNIYTQPVAQW
HDRGEQEVFEYCLEDGSLIRATKDHKFMTVDGQMLPIDEIFERELDL
MRVDNLPN
Nostoc punctiforme: Npu DnaE C-terminal
Split Intein
(SEQ ID NO: 81)
MIKIATRKYLGKQNVYDIGVERDHNFALKNGFIASNCFN
Synthetic: Cfa N-Terminal Split Intein
(SEQ ID NO: 82)
CLSYDTEILTVEYGFLPIGKIVEERIECTVYTVDKNGFVYTQPIAQW
HNRGEQEVFEYCLEDGSIIRATKDHKFMTTDGQMLPIDEIFERGLDL
KQVDGLP
Synthetic: Cfa C-Terminal Split Intein
(SEQ ID NO: 83)
MVKIISRKSLGTQNVYDIGVEKDHNFLLKNGLVASN
Saccharomyces cerevisiae: Vma N-terminal
Split Intein
(SEQ ID NO: 84)
CFAKGTNVLMADGSIECIENIEVGNKVMGKDGRPREVIKLPRGRETM
YSWQKSQHRAHKSDSSREVPELLKFTCNATHELWRTPRSVRRLSRTI
KGVEYFEVITFEMGQKKAPDGRIVELVKEVSKSYPISEGPERANELV
ESYRKASNKAYFEWTIEARDLSLLGSHVRKATYQTYAPILY
Saccharomyces cerevisiae: Vma C-terminal
Split Intein
(SEQ ID NO: 85)
VLLNVLSKCAGSKKFRPAPAAAFARECRGFYFELQELKEDDYYGITL
SDDSDHQFLLANQVWHN
Synechocystis sp. PCC 6803: Ssp DnaE
N-terminal Split Intein
(SEQ ID NO: 86)
CLSFGTEILTVEYGPLPIGKIVSEEINCSVYSVDPEGRVYTQAIAQW
HDRGEQEVLEYELEDGSVIRATSDHRFLTTDYQLLAIEEIFARQLDL
LTLENIKQTEEALDNHRLPFPLLDAGTIK
Synechocystis sp. PCC 6803: Ssp DnaE
C-terminal Split Intein
(SEQ ID NO: 87)
MVKVIGRRSLGVQRIFDIGLPQDHNFLLANGAIAAN
Synthetic: Spy Tag
(SEQ ID NO: 88)
VPTIVMVDAYKRYK
Synthetic: Spy Catcher
(SEQ ID NO: 89)
MVTTLSGLSGEQGPSGDMTTEEDSATHIKFSKRDEDGRELAGATMEL
RDSSGKTISTWISDGHVKDFYLYPGKYTFVETAAPDGYEVATAITFT
VNEQGQVTVNGEATKGDAHTGSSGS
Bacteriophage MS2: MS2 RNA Binding Protein
(SEQ ID NO: 90)
MASNFTQFVLVDNGGTGDVTVAPSNFANGVAEWISSNSRSQAYKVTC
SVRQSSAQNRKYTIKVEVPKVATQTVGGVELPVAAWRSYLNMELTIP
IFATNSDCELIVKAMQGLLKDGNPIPSAIAANSGIY
Bacteriophage MS2: MS2 (N55K) RNA
Binding Protein
(SEQ ID NO: 91)
MASNFTQFVLVDNGGTGDVTVAPSNFANGVAEWISSNSRSQAYKVTCS
VRQSSAQKRKYTIKVEVPKVATQTVGGVELPVAAWRSYLNMELTIPI
FATNSDCELIVKAMQGLLKDGNPIPSAIAANSGIY
Bacteriophage MS2: MS2 (N55K)(V29I) RNA
Binding Protein
(SEQ ID NO: 92)
MASNFTQFVLVDNGGTGDVTVAPSNFANGIAEWISSNSRSQAYKVTC
SVRQSSAQKRKYTIKVEVPKVATQTVGGVELPVAAWRSYLNMELTIP
IFATNSDCELIVKAMQGLLKDGNPIPSAIAANSGIY
Bacteriophage PP7: PP7 RNA Binding Protein
(SEQ ID NO: 93)
KTIVLSVGEATRTLTEIQSTADRQIFEEKVGPLVGRLRLTASLRQNG
AKTAYRVNLKLDQADVVDSGLPKVRYTQVWSHDVTIVANSTEASRKS
LYDLTKSLVATSQVEDLWNLVPLGRS
Bacteriophage Mu: COM RNA Binding Protein
(SEQ ID NO: 94)
MKSIRCKNCNKLLFKADSFDHIEIRCPRCKRHIIMLNACEHPTEKHC
GKREKITHSDETVRY
Synthetic: Zinc Finger ZF6/10
(SEQ ID NO: 95)
STRPGERPFQCRICMRNFSIPNHLARHTRTHTGEKPFQCRICMRNFS
QSAHLKRHLRTHTGEKPFQCRICMRNFSQDVSLVRHLKTHLRQKDGE
RPFQCRICMRNFSSAQALARHTRTHTGEKPFQCRICMRNFSQGGNLT
RHLRTHTGEKPFQCRICMRNFSQHPNLTRHLKTHLRGS
Synthetic: Zinc Finger ZF8/7
(SEQ ID NO: 96)
SRPGERPFQCRICMRNFSTMAVLRRHTRTHTGEKPFQCRICMRNFSR
REVLENHLRTHTGEKPFQCRICMRNFSQTVNLDRHLKTHLRQKDGER
PFQCRICMRNFSKKDHLHRHTRTHTGEKPFQCRICMRNFSQRPHLTN
HLRTHTGEKPFQCRICMRNFSVGASLKRHLKTHLRGS
Synthetic: Zinc Finger ZF9
(SEQ ID NO: 97)
SRPGERPFQCRICMRNFSDKTKLRVHTRTHTGEKPFQCRICMRNFSV
RHNLTRHLRTHTGEKPFQCRICMRNFSQSTSLQRHLKTHLRGF
Synthetic: Zinc Finger MK10
(SEQ ID NO: 98)
SRPGERPFQCRICMRNFSRRHGLDRHTRTHTGEKPFQCRICMRNFSD
HSSLKRHLRTHTGSQKPFQCRICMRNFSVRHNLTRHLRTHTGEKPFQ
CRICMRNFSDHSNLSRHLKTHTGSQKPFQCRICMRNFSQRSSLVRHL
RTHTGEKPFQCRICMRNFSESGHLKRHLRTHLRGS
Synthetic: Zinc Finger 268
(SEQ ID NO: 99)
YACPVESCDRRFSRSDELTRHIRIHTGQKPFQCRICMRNFSRSDHLT
THIRTHTGEKPFACDICGRKFARSDERKRHTKIHLRQKD
Synthetic: Zinc Finger NRE
(SEQ ID NO: 100)
YACPVESCDRRFSQSHDLTKHIRIHTGQKPFQCRICMRNFSDSSKLS
RHIRTHTGEKPFACDICGRKFARLDNRTAHTKIHLRQKD
Synthetic: Zinc Finger 268/NRE
(SEQ ID NO: 101)
YACPVESCDRRFSRSDELTRHIRIHTGQKPFQCRICMRNFSRSDHLT
THIRTHTGEKPFACDICGRKFARSDERKRHTKIHLRQKDGERPYACP
VESCDRRFSQSHDLTKHIRIHTGQKPFQCRICMRNFSDSSKLSRHIR
THTGEKPFACDICGRKFARLDNRTAHTKIHLRQKD
Synthetic: Zinc Finger 268//NRE
(SEQ ID NO: 102)
YACPVESCDRRFSRSDELTRHIRIHTGQKPFQCRICMRNFSRSDHLT
THIRTHTGEKPFACDICGRKFARSDERKRHTKIHLRQKDGGGSERPY
ACPVESCDRRFSQSHDLTKHIRIHTGQKPFQCRICMRNFSDSSKLSR
HIRTHTGEKPFACDICGRKFARLDNRTAHTKIHLRQKD
Synthetic: FokI Zinc Finger Nuclease
17-2 Targeting GFP
(SEQ ID NO: 103)
SRPGERPFQCRICMRNFSTRQNLDTHTRTHTGEKPFQCRICMRNFSR
RDTLERHLRTHTGEKPFQCRICMRNFSRPDALPRHLKTHLRGSQLVK
SELEEKKSELRHKLKYVPHEYIELIEIARNSTQDRILEMKVMEFFMK
VYGYRGKHLGGSRKPDGAIYTVGSPIDYGVIVDTKAYSGGYNLPIGQ
ADEMQRYVEENQTRNKHINPNEWWKVYPSSVTEFKFLFVSGHFKGNY
KAQLTRLNHITNCNGAVLSVEELLIGGEMIKAGTLTLEEVRRKFNNG
EINF
Synthetic: FokI Zinc Finger Nuclease
18-2 Targeting GFP
(SEQ ID NO: 104)
SRPGERPFQCRICMRNFSSPSKLIRHTRTHTGEKPFQCRICMRNFSD
GSNLARHLRTHTGEKPFQCRICMRNFSRVDNLPRHLKTHLRGSQLVK
SELEEKKSELRHKLKYVPHEYIELIEIARNSTQDRILEMKVMEFFMK
VYGYRGKHLGGSRKPDGAIYTVGSPIDYGVIVDTKAYSGGYNLPIGQ
ADEMQRYVEENQTRNKHINPNEWWKVYPSSVTEFKFLFVSGHFKGNY
KAQLTRLNHITNCNGAVLSVEELLIGGEMIKAGTLTLEEVRRKFNNG
EINF
Synthetic: Left FokI Zinc Finger Nuclease
Targeting CCR5
(SEQ ID NO: 105)
MDYKDHDGDYKDHDIDYKDDDDKMAPKKKRKVGIHGVPAAMAERPFQ
CRICMRNFSDRSNLSRHIRTHTGEKPFACDICGRKFAISSNLNSHTK
IHTGSQKPFQCRICMRNFSRSDNLARHIRTHTGEKPFACDICGRKFA
TSGNLTRHTKIHLRGSQLVKSELEEKKSELRHKLKYVPHEYIELIEI
ARNSTQDRILEMKVMEFFMKVYGYRGKHLGGSRKPDGAIYTVGSPID
YGVIVDTKAYSGGYNLPIGQADEMQRYVEENQTRNKHINPNEWWKVY
PSSVTEFKFLFVSGHFKGNYKAQLTRLNHITNCNGAVLSVEELLIGG
EMIKAGTLTLEEVRRKFNNGEINF
Synthetic: Right FokI Zinc Finger Nuclease
Targeting CCR5
(SEQ ID NO: 106)
MDYKDHDGDYKDHDIDYKDDDDKMAPKKKRKVGIHGVPAAMAERPFQ
CRICMRNFSRSDNLSVHIRTHTGEKPFACDICGRKFAQKINLQVHTK
IHTGEKPFQCRICMRNFSRSDVLSEHIRTHTGEKPFACDICGRKFAQ
RNHRTTHTKIHLRGSQLVKSELEEKKSELRHKLKYVPHEYIELIEIA
RNSTQDRILEMKVMEFFMKVYGYRGKHLGGSRKPDGAIYTVGSPIDY
GVIVDTKAYSGGYNLPIGQADEMQRYVEENQTRNKHINPNEWWKVYP
SSVTEFKFLFVSGHFKGNYKAQLTRLNHITNCNGAVLSVEELLIGGE
MIKAGTLTLEEVRRKFNNGEINF
Synthetic: FokI Nuclease Domain
(SEQ ID NO: 107)
QLVKSELEEKKSELRHKLKYVPHEYIELIEIARNSTQDRILEMKVME
FFMKVYGYRGKHLGGSRKPDGAIYTVGSPIDYGVIVDTKAYSGGYNL
PIGQADEMQRYVEENQTRNKHINPNEWWKVYPSSVTEFKFLFVSGHF
KGNYKAQLTRLNHITNCNGAVLSVEELLIGGEMIKAGTLTLEEVRRK
FNNGEINF
Synthetic: AcuI Nuclease Domain
(SEQ ID NO: 108)
VHDHKLELAKLIRNYETNRKECLNSRYNETLLRSDYLDPFFELLGWD
IKNKAGKPTNEREVVLEEALKASASEHSKKPDYTFRLFSERKFFLEA
KKPSVHIESDNETAKQVRRYGFTAKLKISVLSNFEYLVIYDTSVKVD
GDDTFNKARIKKYHYTEYETHFDEICDLLGRESVYSGNFDKEWLSIE
NKINHFSVDTL
Synthetic: Truncated AcuI Nuclease Domain
(SEQ ID NO: 109)
YNETLLRSDYLDPFFELLGWDIKNKAGKPTNEREWLEEALKASASEH
SKKPDYTFRLFSERKFFLEAKKPSVHIESDNETAKQVRRYGFTAKLK
ISVLSNFEYLVIYDTSVKVDGDDT
Escherichia coli: Ferritin
(SEQ ID NO: 110)
MLKPEMIEKLNEQMNLELYSSLLYQQMSAWCSYHTFEGAAAFLRRHA
QEEMTHMQRLFDYLTDTGNLPRINTVESPFAEYSSLDELFQETYKHE
QLITQKINELAHAAMTNQDYPTFNFLQWYVSEQHEEEKLFKSIIDKL
SLAGKSGEGLYFIDKELSTLDTQN
Escherichia coli: Ferritin (H34L)(T64I)
(SEQ ID NO: 111)
MLKPEMIEKLNEQMNLELYSSLLYQQMSAWCSYLTFEGAAAFLRRHA
QEEMTHMQRLFDYLTDIGNLPRINTVESPFAEYSSLDELFQETYKHE
QLITQKINELAHAAMTNQDYPTFNFLQWYVSEQHEEEKLFKSIIDKL
SLAGKSGEGLYFIDKELSTLDTQN
Mus musculus & Synthetic: Light & Heavy
Chain Ferritin Chimera
(SEQ ID NO: 112)
MTSQIRQNYSTEVEAAVNRLVNLHLRASYTYLSLGFFFDRDDVALEG
VGHFFRELAEEKREGAERLLEFQNDRGGRALFQDVQKPSQDEWGKTQ
EAMEAALAMEKNLNQALLDLHALGSARADPHLCDFLESHYLDKEVKL
IKKMGNHLTNLRRVAGPQPAQTGAPQGSLGEYLFERLTLKHDARGGG
GSDYKDDDDKGGGGSRVMTTASPSQVRQNYHQDAEAAINRQINLELY
ASYVYLSMSCYFDRDDVALKNFAKYFLHQSHEEREHAEKLMKLQNQR
GGRIFLQDIKKPDRDDWESGLNAMECALHLEKSVNQSLLELHKLATD
KNDPHLCDFIETYYLSEQVKSIKELGDHVTNLRKMGAPEAGMAEYLF
DKHTLGHGDESTR
Homo sapiens & Synthetic: Light & Heavy
Chain Ferritin Chimera
(SEQ ID NO: 113)
SQIRQNYSTDVEAAVNSLVNLYLQASYTYLSLGFYFDRDDVALEGVS
HFFRELAEEKREGYERLLKMQNQRGGRALFQDIKKPAEDEWGKTPDA
MKAAMALEKKLNQALLDLHALGSARTDPHLCDFLETHFLDEEVKLIK
KMGDHLTNLHRLGGPEAGLGEYLFERLTLKHDARGGGGSDYKDDDDK
GGGGSRVMTTASTSQVRQNYHQDSEAAINRQINLELYASYVYLSMSY
YFDRDDVALKNFAKYFLHQSHEEREHAEKLMKLQNQRGGRIFLQDIK
KPDCDDWESGLNAMECALHLEKNVNQSLLELHKLATDKNDPHLCDFI
ETHYLNEQVKAIKELGDHVTNLRKMGAPESGLAEYLFDKHTLGDSDN
ES
Arabidopsis thaliana: Cry2
(SEQ ID NO: 114)
MKMDKKTIVWFRRDLRIEDNPALAAAAHEGSVFPVFIWCPEEEGQFY
PGRASRWWMKQSLAHLSQSLKALGSDLTLIKTHNTISAILDCIRVTG
ATKVVFNHLYDPVSLVRDHTVKEKLVERGISVQSYNGDLLYEPWEIY
CEKGKPFTSFNSYWKKCLDMSIESVMLPPPWRLMPITAAAEAIWACS
IEELGLENEAEKPSNALLTRAWSPGWSNADKLLNEFIEKQLIDYAKN
SKKWGNSTSLLSPYLHFGEISVRHVFQCARMKQIIWARDKNSEGEES
ADLFLRGIGLREYSRYICFNFPFTHEQSLLSHLRFFPWDADVDKFKA
WRQGRTGYPLVDAGMRELWATGWMHNRIRVIVSSFAVKFLLLPWKWG
MKYFWDTLLDADLECDILGWQYISGSIPDGHELDRLDNPALQGAKYD
PEGEYIRQWLPELARLPTEWIHHPWDAPLTVLKASGVELGTNYAKPI
VDIDTARELLAKAISRTREAQIMIGAAPDEIVADSFEALGANTIKEP
GLCPSVSSNDQQVPSAVRYNGSAAVKPEEEEERDMKKSRGFDERELF
STAESSSSSSVFFVSQSCSLASEGKNLEGIQDSSDQITTSLGKNGCK
Arabidopsis thaliana: CIBN
(SEQ ID NO: 115)
MNGAIGGDLLLNFPDMSVLERQRAHLKYLNPTFDSPLAGFFADSSMI
TGGEMDSYLSTAGLNLPMMYGETTVEGDSRLSISPETTLGTGNFKAA
KFDTETKDCNEAAKKMTMNRDDLVEEGEEEKSKITEQNNGSTKSIKK
MKHKAKKEENNFSNDSSKVTKELEKTDYI
Synthetic: LoV2-Ja
(SEQ ID NO: 116)
SLATTLERIEKNFVITDPRLPDNPIIFASDSFLQLTEYSREEILGRN
CRFLQGPETDRATVRKIRDAIDNQTEVTVQLINYTKSGKKFWNLFHL
QPMRDQKGDVQYFIGVQLDGTEHVRDAAEREGVMLIKKTAENIDEAA
KEL
Homo sapiens: Full Length WT ADAR2
(SEQ ID NO: 117)
DIEDEENMSSSSTDVKENRNLDNVSPKDGSTPGPGEGSQLSNGGGGG
PGRKRPLEEGSNGHSKYRLKKRRKTPGPVLPKNALMQLNEIKPGLQY
TLLSQTGPVHAPLFVMSVEVNGQVFEGSGPTKKKAKLHAAEKALRSF
VQFPNASEAHLAMGRTLSVNTDFTSDQADFPDTLFNGFETPDKAEPP
FYVGSNGDDSFSSSGDLSLSASPVPASLAQPPLPVLPPFPPPSGKNP
VMILNELRPGLKYDFLSESGESHAKSFVMSVWDGQFFEGSGRNKKLA
KARAAQSALAAIFNLHLDQTPSRQPIPSEGLQLHLPQVLADAVSRLV
LGKFGDLTDNFSSPHARRKVLAGVVMTTGTDVKDAKVISVSTGTKCI
NGEYMSDRGLALNDCHAEIISRRSLLRFLYTQLELYLNNKDDQKRSI
FQKSERGGFRLKENVQFHLYISTSPCGDARIFSPHEPILEGSRSYTQ
AGVQWCNHGSLQPRPPGLLSDPSTSTFQGAGTTEPADRHPNRKARGQ
LRTKIESGEGTIPVRSNASIQTWDGVLQGERLLTMSCSDKIARWNWG
IQGSLLSIFVEPIYFSSIILGSLYHGDHLSRAMYQRISNIEDLPPLY
TLNKPLLSGISNAEARQPGKAPNFSVNWTVGDSAIEVINATTGKDEL
GRASRLCKHALYCRWMRVHGKVPSHLLRSKITKPNVYHESKLAAKEY
QAAKARLFTAFIKAGLGAVWEKPTEQDQFSLTP
Homo sapiens: Full Length WT ADAR2 (E488Q)
(SEQ ID NO: 118)
DIEDEENMSSSSTDVKENRNLDNVSPKDGSTPGPGEGSQLSNGGGGG
PGRKRPLEEGSNGHSKYRLKKRRKTPGPVLPKNALMQLNEIKPGLQY
TLLSQTGPVHAPLFVMSVEVNGQVFEGSGPTKKKAKLHAAEKALRSF
VQFPNASEAHLAMGRTLSVNTDFTSDQADFPDTLFNGFETPDKAEPP
FYVGSNGDDSFSSSGDLSLSASPVPASLAQPPLPVLPPFPPPSGKNP
VMILNELRPGLKYDFLSESGESHAKSFVMSVWDGQFFEGSGRNKKLA
KARAAQSALAAIFNLHLDQTPSRQPIPSEGLQLHLPQVLADAVSRLV
LGKFGDLTDNFSSPHARRKVLAGVVMTTGTDVKDAKVISVSTGTKCI
NGEYMSDRGLALNDCHAEIISRRSLLRFLYTQLELYLNNKDDQKRSI
FQKSERGGFRLKENVQFHLYISTSPCGDARIFSPHEPILEGSRSYTQ
AGVQWCNHGSLQPRPPGLLSDPSTSTFQGAGTTEPADRHPNRKARGQ
LRTKIESGQGTIPVRSNASIQTWDGVLQGERLLTMSCSDKIARWNWG
IQGSLLSIFVEPIYFSSIILGSLYHGDHLSRAMYQRISNIEDLPPLY
TLNKPLLSGISNAEARQPGKAPNFSVNWTVGDSAIEVINATTGKDEL
GRASRLCKHALYCRWMRVHGKVPSHLLRSKITKPNVYHESKLAAKEY
QAAKARLFTAFIKAGLGAWVEKPTEQDQFSLTP
Homo sapiens: Truncated WT ADAR2
(SEQ ID NO: 119)
VLADAVSRLVLGKFGDLTDNFSSPHARRKVLAGWMTTGTDVKDAKVI
SVSTGTKCINGEYMSDRGLALNDCHAEIISRRSLLRFLYTQLELYLN
NKDDQKRSIFQKSERGGFRLKENVQFHLYISTSPCGDARIFSPHEPI
LEGSRSYTQAGVQWCNHGSLQPRPPGLLSDPSTSTFQGAGTTEPADR
HPNRKARGQLRTKIESGEGTIPVRSNASIQTWDGVLQGERLLTMSCS
DKIARWNWGIQGSLLSIFVEPIYFSSIILGSLYHGDHLSRAMYQRIS
NIEDLPPLYTLNKPLLSGISNAEARQPGKAPNFSVNWTVGDSAIEVI
NATTGKDELGRASRLCKHALYCRWMRVHGKVPSHLLRSKITKPNVYH
ESKLAAKEYQAAKARLFTAFIKAGLGAVWEKPTEQDQFSLTP
Homo sapiens: Truncated WT ADAR2 (E488Q)
(SEQ ID NO: 120)
VLADAVSRLVLGKFGDLTDNFSSPHARRKVLAGVMTTGTDVKDAKVI
SVSTGTKCINGEYMSDRGLALNDCHAEIISRRSLLRFLYTQLELYLN
NKDDQKRSIFQKSERGGFRLKENVQFHLYISTSPCGDARIFSPHEPI
LEGSRSYTQAGVQWCNHGSLQPRPPGLLSDPSTSTFQGAGTTEPADR
HPNRKARGQLRTKIESGQGTIPVRSNASIQTWDGVLQGERLLTMSCS
DKIARWNWGIQGSLLSIFVEPIYFSSIILGSLYHGDHLSRAMYQRIS
NIEDLPPLYTLNKPLLSGISNAEARQPGKAPNFSVNWTVGDSAIEVI
NATTGKDELGRASRLCKHALYCRWMRVHGKVPSHLLRSKITKPNVYH
ESKLAAKEYQAAKARLFTAFIKAGLGAWWEKPTEQDQFSLTP
Homo sapiens & Synthetic: MS2-ADAR1 Deaminase
Domain-Nuclear Exclusion Signal
(SEQ ID NO: 121)
MASNFTQFVLVDNGGTGDVTVAPSNFANGIAEWISSNSRSQAYKVTC
SVRQSSAQNRKYTIKVEVPKGAWRSYLNMELTIPIFATNSDCELIVK
AMQGLLKDGNPIPSAIAANSGIYGGSGSGAGSGSPAGGGAPGSGGGS
KAERMGFTEVTPVTGASLRRTMLLLSRSPEAQPKTLPLTGSTFHDQI
AMLSHRCFNTLTNSFQPSLLGRKILAAIIMKKDSEDMGVWVSLGTGN
RCVKGDSLSLKGETVNDCHAEIISRRGFIRFLYSELMKYNSQTAKDS
IFEPAKGGEKLQIKKTVSFHLYISTAPCGDGALFDKSCSDRAMESTE
SRHYPVFENPKQGKLRTKVENGEGTIPVESSDIVPTWDGIRLGERLR
TMSCSDKILRWNVLGLQGALLTHFLQPIYLKSVTLGYLFSQGHLTRA
ICCRVTRDGSAFEDGLRHPFIVNHPKVGRVSIYDSKRQSGKTKETSV
NWCLADGYDLEILDGTRGTVDGPRNELSRVSKKNIFLLFKKLCSFRY
RRDLLRLSYGEAKKAARDYETAKNYFKKGLKDMGYGNWISKPQEEKN
FYLCPVGSGSGSLPPLERLT
Homo sapiens & Synthetic: MS2-ADAR1 Deaminase
Domain (E1008Q)-
Nuclear Exclusion Signal
(SEQ ID NO: 122)
MASNFTQFVLVDNGGTGDVTVAPSNFANGIAEWISSNSRSQAYKVTC
SVRQSSAQNRKYTIKVEVPKGAWRSYLNMELTIPIFATNSDCELIVK
AMQGLLKDGNPIPSAIAANSGIYGGSGSGAGSGSPAGGGAPGSGGGS
KAERMGFTEVTPVTGASLRRTMLLLSRSPEAQPKTLPLTGSTFHDQI
AMLSHRCFNTLTNSFQPSLLGRKILAAIIMKKDSEDMGVWVSLGTGN
RCVKGDSLSLKGETVNDCHAEIISRRGFIRFLYSELMKYNSQTAKDS
IFEPAKGGEKLQIKKTVSFHLYISTAPCGDGALFDKSCSDRAMESTE
SRHYPVFENPKQGKLRTKVENGQGTIPVESSDIVPTWDGIRLGERLR
TMSCSDKILRWNVLGLQGALLTHFLQPIYLKSVTLGYLFSQGHLTRA
ICCRVTRDGSAFEDGLRHPFIVNHPKVGRVSIYDSKRQSGKTKETSV
NWCLADGYDLEILDGTRGTVDGPRNELSRVSKKNIFLLFKKLCSFRY
RRDLLRLSYGEAKKAARDYETAKNYFKKGLKDMGYGNWISKPQEEKN
FYLCPVGSGSGSLPPLERLTL
Ruminococcus flavefaciens: RfxCas13d (CasRx)
(SEQ ID NO: 123)
EASIEKKKSFAKGMGVKSTLVSGSKVYMTTFAEGSDARLEKIVEGDS
IRSVNEGEAFSAEMADKNAGYKIGNAKFSHPKGYAVVANNPLYTGPV
QQDMLGLKETLEKRYFGESADGNDNICIQVIHNILDIEKILAEYITN
AAYAVNNISGLDKDIIGFGKFSTVYTYDEFKDPEHHRAAFNNNDKLI
NAIKAQYDEFDNFLDNPRLGYFGQAFFSKEGRNYIINYGNECYDILA
LLSGLRHWVVHNNEEESRISRTWLYNLDKNLDNEYISTLNYLYDRIT
NELTNSFSKNSAANVNYIAETLGINPAEFAEQYFRFSIMKEQKNLGF
NITKLREVMLDRKDMSEIRKNHKVFDSIRTKVYTMMDFVIYRYYIEE
DAKVAAANKSLPDNEKSLSEKDIFVINLRGSFNDDQKDALYYDEANR
IWRKLENIMHNIKEFRGNKTREYKKKDAPRLPRILPAGRDVSAFSKL
MYALTMFLDGKEINDLLTTLINKFDNIQSFLKVMPLIGVNAKFVEEY
AFFKDSAKIADELRLIKSFARMGEPIADARRAMYIDAIRILGTNLSY
DELKALADTFSLDENGNKLKKGKHGMRNFIINNVISNKRFHYLIRYG
DPAHLHEIAKNEAVKFVLGRIADIQKKQGQNGKNQIDRYYETCIGKD
KGKSVSEKVDALTKIITGMNYDQFDKKRSVIEDTGRENAEREKFKKI
ISLYLTVIYHILKNIVNINARYVIGFHCVERDAQLYKEKGYDINLKK
LEEKGFSSVTKLCAGIDETAPDKRKDVEKEMAERAKESIDSLESANP
KLYANYIKYSDEKKAEEFTRQINREKAKTALNAYLRNTKWNVIIRED
LLRIDNKTCTLFRNKAVHLEVARYVHAYINDIAEVNSYFQLYHYIMQ
RIIMNERYEKSSGKVSEYFDAVNDEKKYNDRLLKLLCVPFGYCIPRF
KNLSIEALFDRNEAAKFDKEKKKVSGNSGSG
Ruminococcus flavefaciens & Synthetic: dead
RfxCas13d (dCasRx)
(SEQ ID NO: 124)
EASIEKKKSFAKGMGVKSTLVSGSKVYMTTFAEGSDARLEKIVEGDS
IRSVNEGEAFSAEMADKNAGYKIGNAKFSHPKGYAVVANNPLYTGPV
QQDMLGLKETLEKRYFGESADGNDNICIQVIHNILDIEKILAEYITN
AAYAVNNISGLDKDIIGFGKFSTVYTYDEFKDPEHHRAAFNNNDKLI
NAIKAQYDEFDNFLDNPRLGYFGQAFFSKEGRNYIINYGNECYDILA
LLSGLAHWWANNEEESRISRTWLYNLDKNLDNEYISTLNYLYDRITN
ELTNSFSKNSAANVNYIAETLGINPAEFAEQYFRFSIMKEQKNLGFN
ITKLREVMLDRKDMSEIRKNHKVFDSIRTKVYTMMDFVIYRYYIEED
AKVAAANKSLPDNEKSLSEKDIFVINLRGSFNDDQKDALYYDEANRI
WRKLENIMHNIKEFRGNKTREYKKKDAPRLPRILPAGRDVSAFSKLM
YALTMFLDGKEINDLLTTLINKFDNIQSFLKVMPLIGVNAKFVEEYA
FFKDSAKIADELRLIKSFARMGEPIADARRAMYIDAIRILGTNLSYD
ELKALADTFSLDENGNKLKKGKHGMRNFIINNVISNKRFHYLIRYGD
PAHLHEIAKNEAWKFVLGRIADIQKKQGQNGKNQIDRYYETCIGKDK
GKSVSEKVDALTKIITGMNYDQFDKKRSVIEDTGRENAEREKFKKII
SLYLTVIYHILKNIVNINARYVIGFHCVERDAQLYKEKGYDINLKKL
EEKGFSSVTKLCAGIDETAPDKRKDVEKEMAERAKESIDSLESANPK
LYANYIKYSDEKKAEEFTRQINREKAKTALNAYLRNTKWNVIIREDL
LRIDNKTCTLFANKAVALEVARYVHAYINDIAEVNSYFQLYHYIMQR
IIMNERYEKSSGKVSEYFDAVNDEKKYNDRLLKLLCVPFGYCIPRFK
NLSIEALFDRNEAAKFDKEKKKVSGNSGSGPKKKRKVAAAYPYDVPD
YA
Prevotella sp. P5-125: PspCas13b
(SEQ ID NO: 125)
MNIPALVENQKKYFGTYSVMAMLNAQTVLDHIQKVADIEGEQNENNE
NLWFHPVMSHLYNAKNGYDKQPEKTMFIIERLQSYFPFLKIMAENQR
EYSNGKYKQNRVEVNSNDIFEVLKRAFGVLKMYRDLTNHYKTYEEKL
NDGCEFLTSTEQPLSGMINNYYTVALRNMNERYGYKTEDLAFIQDKR
FKFVKDAYGKKKSQVNTGFFLSLQDYNGDTQKKLHLSGVGIALLICL
FLDKQYINIFLSRLPIFSSYNAQSEERRIIIRSFGINSIKLPKDRIH
SEKSNKSVAMDMLNEVKRCPDELFTTLSAEKQSRFRIISDDHNEVLM
KRSSDRFVPLLLQYIDYGKLFDHIRFHVNMGKLRYLLKADKTCIDGQ
TRVRVIEQPLNGFGRLEEAETMRKQENGTFGNSGIRIRDFENMKRDD
ANPANYPYIVDTYTHYILENNKVEMFINDKEDSAPLLPVIEDDRYVV
KTIPSCRMSTLEIPAMAFHMFLFGSKKTEKLIVDVHNRYKRLFQAMQ
KEEVTAENIASFGIAESDLPQKILDLISGNAHGKDVDAFIRLTVDDM
LTDTERRIKRFKDDRKSIRSADNKMGKRGFKQISTGKLADFLAKDIV
LFQPSVNDGENKITGLNYRIMQSAIAVYDSGDDYEAKQQFKLMFEKA
RLIGKGTTEPHPFLYKVFARSIPANAVEFYERYLIERKFYLTGLSNE
IKKGNRVDVPFIRRDQNKWKTPAMKTLGRIYSEDLPVELPRQMFDNE
IKSHLKSLPQMEGIDFNNANVTYLIAEYMKRVLDDDFQTFYQWNRNY
RYMDMLKGEYDRKGSLQHCFTSVEEREGLWKERASRTERYRKQASNK
IRSNRQMRNASSEEIETILDKRLSNSRNEYQKSEKVIRRYRVQDALL
FLLAKKTLTELADFDGERFKLKEIMPDAEKGILSEIMPMSFTFEKGG
KKYTITSEGMKLKNYGDFFVLASDKRIGNLLELVGSDIVSKEDIMEE
FNKYDQCRPEISSIVFNLEKWAFDTYPELSARVDREEKVDFKSILKI
LLNNKNINKEQSDILRKIRNAFDHNNYPDKGVEIKALPEIAMSIKKA
FGEYAIMKGSLQ
Synthetic: L17E
(SEQ ID NO: 126)
IWLTALKFLGKHAAKHEAKQQLSKL
Synthetic: L17E-Transmembrane
(SEQ ID NO: 127)
IWLTALKFLGKHAAKHEAKQQLSKLNAVGQDTQEVIVPHSLPFKVVV
ISAILALVVLTIISLHLIMLWQKKPR
Synthetic: KALA
(SEQ ID NO: 128)
WEAKLAKALAKALAKHLAKALAKALKACEA
Synthetic: KALA-Transmembrane
(SEQ ID NO: 129)
WEAKLAKALAKALAKHLAKALAKALKACEANAVGQDTQEVIVVPHSL
PFKVWISAILALVVLTIISLIILIMLWQKKPR
Synthetic: Vectofusin
(SEQ ID NO: 130)
KKALLHAALAHLLALAHHLLALLKKA
Synthetic: Vectofusin-Transmembrane
(SEQ ID NO: 131)
KKALLHAALAHLLALAHHLLALLKKANAVGQDTQEVIVVPHSLPFKW
VISAILALVLTIISLIILIMLWQKKPR
Synthetic: Transmembrane Domain
(SEQ ID NO: 132)
NAVGQDTQEVIVVPHSLPFKVVVISAILALVVLTIISLIILIMLWQK
KPR
Lactococcus lactis: Nisin A
(SEQ ID NO: 133)
ITSISLCTPGCKTGALMGCNMKTATCHCSIHVSK
Lactococcus lactis NIZO 22186: Nisin Z
(SEQ ID NO: 134)
ITSISLCTPGCKTGALMGCNMKTATCNCSIHVSK
Lactococcus lactis subsp. lactis F10: Nisin F
(SEQ ID NO: 135)
ITSISLCTPGCKTGALMGCNMKTATCNCSVHVSK
Lactococcus lactis 61-14: Nisin Q
(SEQ ID NO: 136)
ITSISLCTPGCKTGVLMGCNLKTATCNCSVHVSK
Streptococcus hyointestinalis: Nisin H
(SEQ ID NO: 137)
FTSISMCTPGCKTGALMTCNYKTATCHCSIKVSK
Streptococcus uberis: Nisin U
(SEQ ID NO: 138)
ITSKSLCTPGCKTGILMTCPLKTATCGCHFG
Streptococcus uberis: Nisin U2
(SEQ ID NO: 139)
VTSKSLCTPGCKTGILMTCPLKTATCGCHFG
Streptococcus galloyticus subsp.
pasteurianus: Nisin P
(SEQ ID NO: 140)
VTSKSLCTPGCKTGILMTCAIKTATCGCHFG
L. lactis NZ9800: Nisin A S29A
(SEQ ID NO: 141)
ITSISLCTPGCKTGALMGCNMKTATCHCAIHVSK
L. lactis NZ9800: Nisin A S29D
(SEQ ID NO: 142)
ITSISLCTPGCKTGALMGCNMKTATCHCDIHVSK
L. lactis NZ9800: Nisin A S29E
(SEQ ID NO: 143)
ITSISLCTPGCKTGALMGCNMKTATCHCEIHVSK
L. lactis NZ9800: Nisin A S29G
(SEQ ID NO: 144)
ITSISLCTPGCKTGALMGCNMKTATCHCGIHVSK
L. lactis NZ9800: Nisin A K22T
(SEQ ID NO: 145)
ITSISLCTPGCKTGALMGCNMTTATCHCSIHVSK
L. lactis NZ9800: Nisin A N20P
(SEQ ID NO: 146)
ITSISLCTPGCKTGALMGCPMKTATCHCSIHVSK
L. lactis NZ9800: Nisin A M21V
(SEQ ID NO: 147)
ITSISLCTPGCKTGALMGCNVKTATCHCSIHVSK
L. lactis NZ9800: Nisin A K22S
(SEQ ID NO: 148)
ITSISLCTPGCKTGALMGCNMSTATCHCSIHVSK
L. lactis NZ9800: Nisin Z N20K
(SEQ ID NO: 149)
ITSISLCTPGCKTGALMGCKMKTATCNCSIHVSK
L. lactis NZ9800: Nisin Z M21K
(SEQ ID NO: 150)
ITSISLCTPGCKTGALMGCNKKTATCNCSIHVSK
Relevant RNA Sequences (5′-3′)
Synthetic: MS2 Stem Loop spCas9
Scaffold RNA for sgRNA with
Terminator Example 1
(SEQ ID NO: 151)
GUUUUAGAGCUAGGCCAACAUGAGGAUCACCCAUGUCUGCAGGGCCU
AGCAAGUUAAAAUAAGGCUAGUCCGUUAUCAACUUGGCCAACAUGAG
GAUCACCCAUGUCUGCAGGGCCAAGUGGCACCGAGUCGGUGCUUUUU
UU
Synthetic: MS2 Stem Loop spCas9
Scaffold RNA for sgRNA with
Terminator Example 2
(SEQ ID NO: 152)
GUUUUAGAGCUAGGCCAACAUGAGGAUCACCCAUGUCUGCAGGGCCU
AGCAAGUUAAAAUAAGGCUAGUCCGUUAUCAACUUGGCCAACAUGAG
GAUCACCCAUGUCUGCAGGGCCAAGUGGCACCGAGUCGGUGCGGGAG
CACAUGAGGAUCACCCAUGUGCGACUCCCACAGUCACUGGGGAGUCU
UCCCUUUUUUU
Synthetic: MS2 Stem Loop spCas9
Scaffold RNA for sgRNA with
Terminator Example 3
(SEQ ID NO: 153)
GUUUAAGAGCUAUGCUGGAAACAGCAUAGCAAGUUUAAAUAAGGCUA
GUCCGUUAUCAACUUGAAAAAGUGGCACCGAGUCGGUGCGGGAGCAC
AUGAGGAUCACCCAUGUGCGACUCCCACAGUCACUGGGGAGUCUUCC
CUUUUUUU
Synthetic: 4xMS2 Stem Loop RNA Scaffold Example
(SEQ ID NO: 154)
UUCUAGAUCAUCGAAACAUGAGGAUCACCCAUAUCUGCAGUCGACAU
CGAAACAUGAGGAUCACCCAUGUCUGCAGUCGACAUCGAAACAUGAG
GAUCACCCAUGUCUGCAGUCGACAUCGAAACAUGAGGAUCACCCAUG
UCUGCAGUCGACAUCGAAAUCGAUAAGCUUCAGAUCAGAUCCUAG
Synthetic: MS2 Stem Loop Example 1
(SEQ ID NO: 155)
ACAUGAGGAUCACCCAUGU
Synthetic: MS2 Stem Loop Example 2
(SEQ ID NO: 156)
ACAUGAGGAUCACCCAUAU
Synthetic: MS2 Stem Loop Example 3
(SEQ ID NO: 157)
CCACAGUCACUGGG
Synthetic: 2xMS2 Stem Loop Example
(SEQ ID NO: 158)
ACAUGAGGAUCACCCAUGUCUGCAGGGCCUAGCAAGUUAAAAUAAGG
CUAGUCCGUUAUCAACUUGGCCAACAUGAGGAUCACCCAUGU
Synthetic: 2xPP7 Stem Loop spCas9
Scaffold RNA for sgRNA with
Terminator Example
(SEQ ID NO: 159)
GUUUUAGAGCUAGGCCGGAGCAGACGAUAUGGCGUCGCUCCGGCCUA
GCAAGUUAAAAUAAGGCUAGUCCGUUAUCAACUUGGCCGGAGCAGAC
GAUAUGGCGUCGCUCCGGCCAAGUGGCACCGAGUCGGUGCUUUUUUU
Synthetic: PP7 Stem Loop Example
(SEQ ID NO: 160)
GCCGGAGCAGACGAUAUGGCGUCGCUCCGGCC
Synthetic: COM Stem Loop spCas9 Scaffold
RNA for sgRNA with
Terminator Example
(SEQ ID NO: 161)
GUUUAAGAGCUAUGCUGGAAACAGCAUAGCAAGUUUAAAUAAGGCUA
GUCCGUUAUCAACUUGAAAAAGUGGCACCGAGUCGGUGCCUGAAUGC
CUGCGAGCAUCUUUUUUU
Synthetic: COM Stem Loop Example
(SEQ ID NO: 162)
CUGAAUGCCUGCGAGCAUC
Synthetic ZKSCAN1 Circular Splice RNA:
Upstream Intron
(SEQ ID NO: 163)
AGUGACAGUGGAGAUUGUACAGUUUUUUCCUCGAUUUGUCAGGAUUU
UUUUUUUUUGACGGAGUUUAACUUCUUGUCUCCCAGGUAGGAAGUGC
AGUGGCGUAAUCUCGGCUCACUACAACCUCCACCUCCUGGGUUCAAG
CGUUUCUCCUGCCUCAGCUUUCCGAGUAGCUGGGAUUACAGGCGCCU
GCCACCAUGCCCUGCUGACUUUUGUAUUUUUAGUAGAGACGGGGUUU
CACCAUGUUGGCCAGGCUGGUCUUGAACUCCUGACCGCAGGCGAUUG
GCCUGCCUCGGCCUCCCAAAGUGCUGAGAUUACAGGCGUGAGCCACC
ACCCCCGGCCUCAGGAGCGUUCUGAUAGUGCCUCGAUGUGCUGCCUC
CUAUAAAGUGUUAGCAGCACAGAUCACUUUUUGUAAAGGUACGUACU
AAUGACUUUUUUUUUAUACUUCAGG
Synthetic ZKSCAN1 Circular Splice RNA:
Downstream Intron
(SEQ ID NO: 164)
UAAGAAGCAAGGUUUCAUUUAGGGGAAGGGAAAUGAUUCAGGACGAG
AGUCUUUGUGCUGCUGAGUGCCUGUGAUGAAGAAGCAUGUUAGUCCU
GGGCAACGUAGCGAGACCCCAUCUCUACAAAAAAUAGAAAAAUUAGC
CAGGUAUAGUGGCGCACACCUGUGAUUCCAGCUACGCAGGAGGCUGA
GGUGGGAGGAUUGCUUGAGCCCAGGAGGUUGAGGCUGCAGUGAGCUG
UAAUCAUGCCACUACUCCAACCUGGGCAACACAGCAAGGACCCUGUC
UCAAAAGCUACUUACAGAAAAGAAUUAGGCUCGGCACGGUAGCUCAC
ACCUGUAAUCCCAGCACUUUGGGAGGCUGAGGCGGGCAGAUCACUUG
AGGUCAGGAGUUUGAGACCAGCCUGGCCAACAUGGUGAAACCUUGUC
UCUACUAAAAAUAUGAAAAUUAGCCAGGCAUGGUGGCACAUUCCUGU
AAUCCCAGCUACUCGGGAGGCUGAGGCAGGAGAAUCACUUGAACCCA
GGAGGUGGAGGUUGCAGUAAGCCGAGAUCGUACCACUGUGCUCUAGC
CUUGGUGACAGAGCGAGACUGUCUUAAAAAAAAAAAAAAAAAAAAAA
GAAUUAAUUAAAAAUUUAAAAAAAAAUGAAAAAAAGCUGCAUGCUUG
UUUUUUGUUUUUAGUUAUUCUACAUUGUUGUCAUUAUUACCAAAUAU
UGGGGAAAAUACAACUUACAGACCAAUCUCAGGAGUUAAAUGUUACU
ACGAAGGCAAAUGAACUAUGCGUAAUGAACCUGGUAGGCAUUAG
Homo sapiens Beta-globin and Immunoglobulin
Heavy Chain Genes: Linear
Splice RNA Intron
(SEQ ID NO: 165)
GUAAGUAUCAAGGUUACAAGACAGGUUUAAGGAGACCAAUAGAAACU
GGGCUUGUCGAGACAGAGAAGACUCUUGCGUUUCUGAUAGGCACCUA
UUGGUCUUACUGACAUCCACUUUGCCUUUCUCUCCACAG
Relevant DNA Sequences (5′-3′)
Synthetic: Zinc Finger ZF6/10 Binding Site
(SEQ ID NO: 166)
GAAGAAGCTGCAGGAGGT
Synthetic: Zinc Finger ZF8/7 Binding Site
(SEQ ID NO: 167)
GCTGGAGGGGAAGTGGTC
Synthetic: Zinc Finger ZF6/10 & ZF8/7
Binding Site
(SEQ ID NO: 168)
GAAGAAGCTGCAGGAGGTGCTGGAGGGGAAGTGGTC
Synthetic: Zinc Finger ZF6/10 & ZF8/7
Binding Site 8x Repeat Example
(SEQ ID NO: 169)
TGAAGAAGCTGCAGGAGGTGCTGGAGGGGAAGTGGTCCGGATCTTGA
AGAAGCTGCAGGAGGTGCTGGAGGGGAAGTGGTCCGGATCTTGAAGA
AGCTGCAGGAGGTGCTGGAGGGGAAGTGGTCCGGATCTTGAAGAAGC
TGCAGGAGGTGCTGGAGGGGAAGTGGTCCGGATCTTGAAGAAGCTGC
AGGAGGTGCTGGAGGGGAAGTGGTCCGGATCTTGAAGAAGCTGCAGG
AGGTGCTGGAGGGGAAGTGGTCCGGATCTTGAAGAAGCTGCAGGAGG
TGCTGGAGGGGAAGTGGTCCGGATCTTGAAGAAGCTGCAGGAGGTGC
TGGAGGGGAAGTGGTCC
Synthetic: Zinc Finger ZF9 Binding Site
(SEQ ID NO: 170)
GTAGATGGA
Synthetic: Zinc Finger MK10 Binding Site
(SEQ ID NO: 171)
CGGCGTAGCCGATGTCGCGC
Synthetic: Zinc Finger 268 Binding Site
(SEQ ID NO: 172)
AAGGGTTCA
Synthetic: Zinc Finger NRE Binding Site
(SEQ ID NO: 173)
GCGTGGGCG
Synthetic: Zinc Finger 268/NRE or
268//NRE Binding Site Example 1
(SEQ ID NO: 174)
AAGGGTTCAGCGTGGGCG
Synthetic: Zinc Finger 268/NRE or
268//NRE Binding Site Example 2
(SEQ ID NO: 175)
AAGGGTTCAGGCGTGGGCG
Synthetic: Zinc Finger 268/NRE or
268//NRE Binding Site Example 3
(SEQ ID NO: 176)
AAGGGTTCAGTGCGTGGGCG
Synthetic: FokI Zinc Finger Nuclease
17-2 & 18-2 Binding Site in GFP
(SEQ ID NO: 177)
GATCCGCCACAACATCGAGGACGGCA
Human codon optimized Streptococcus pyogenes
Cas9 (spCas9) NLS
(SEQ ID NO: 178)
ATGGACAAGAAGTACAGCATCGGCCTGGACATCGGC
ACCAACTCTGTGGGCTGGGC
CGTGATCACCGACGAGTACAAGGTGCCCAGCAAGAAATTCA
AGGTGCTGGGCAACACCGACCGGCACAGCATCAAGAAGAACCTGATC
GGAGCCCTGCTGTTCGACAGCGGCGAAACAGCCGAGGCCACCCGGCT
GAAGAGAACCGCCAGAAGAAGATACACCAGACGGAAGAACCGGATCT
GCTATCTGCAAGAGATCTTCAGCAACGAGATGGCCAAGGTGGACGAC
AGCTTCTTCCACAGACTGGAAGAGTCCTTCCTGGTGGAAGAGGATAA
GAAGCACGAGCGGCACCCCATCTTCGGCAACATCGTGGACGAGGTGG
CCTACCACGAGAAGTACCCCACCATCTACCACCTGAGAAAGAAACTG
GTGGACAGCACCGACAAGGCCGACCTGCGGCTGATCTATCTGGCCCT
GGCCCACATGATCAAGTTCCGGGGCCACTTCCTGATCGAGGGCGACC
TGAACCCCGACAACAGCGACGTGGACAAGCTGTTCATCCAGCTGGTG
CAGACCTACAACCAGCTGTTCGAGGAAAACCCCATCAACGCCAGCGG
CGTGGACGCCAAGGCCATCCTGTCTGCCAGACTGAGCAAGAGCAGAC
GGCTGGAAAATCTGATCGCCCAGCTGCCCGGCGAGAAGAAGAATGGC
CTGTTCGGAAACCTGATTGCCCTGAGCCTGGGCCTGACCCCCAACTT
CAAGAGCAACTTCGACCTGGCCGAGGATGCCAAACTGCAGCTGAGCA
AGGACACCTACGACGACGACCTGGACAACCTGCTGGCCCAGATCGGC
GACCAGTACGCCGACCTGTTTCTGGCCGCCAAGAACCTGTCCGACGC
CATCCTGCTGAGCGACATCCTGAGAGTGAACACCGAGATCACCAAGG
CCCCCCTGAGCGCCTCTATGATCAAGAGATACGACGAGCACCACCAG
GACCTGACCCTGCTGAAAGCTCTCGTGCGGCAGCAGCTGCCTGAGAA
GTACAAAGAGATTTTCTTCGACCAGAGCAAGAACGGCTACGCCGGCT
ACATTGACGGCGGAGCCAGCCAGGAAGAGTTCTACAAGTTCATCAAG
CCCATCCTGGAAAAGATGGACGGCACCGAGGAACTGCTCGTGAAGCT
GAACAGAGAGGACCTGCTGCGGAAGCAGCGGACCTTCGACAACGGCA
GCATCCCCCACCAGATCCACCTGGGAGAGCTGCACGCCATTCTGCGG
CGGCAGGAAGATTTTTACCCATTCCTGAAGGACAACCGGGAAAAGAT
CGAGAAGATCCTGACCTTCCGCATCCCCTACTACGTGGGCCCTCTGG
CCAGGGGAAACAGCAGATTCGCCTGGATGACCAGAAAGAGCGAGGAA
ACCATCACCCCCTGGAACTTCGAGGAAGTGGTGGACAAGGGCGCTTC
CGCCCAGAGCTTCATCGAGCGGATGACCAACTTCGATAAGAACCTGC
CCAACGAGAAGGTGCTGCCCAAGCACAGCCTGCTGTACGAGTACTTC
ACCGTGTATAACGAGCTGACCAAAGTGAAATACGTGACCGAGGGAAT
GAGAAAGCCCGCCTTCCTGAGCGGCGAGCAGAAAAAGGCCATCGTGG
ACCTGCTGTTCAAGACCAACCGGAAAGTGACCGTGAAGCAGCTGAAA
GAGGACTACTTCAAGAAAATCGAGTGCTTCGACTCCGTGGAAATCTC
CGGCGTGGAAGATCGGTTCAACGCCTCCCTGGGCACATACCACGATC
TGCTGAAAATTATCAAGGACAAGGACTTCCTGGACAATGAGGAAAAC
GAGGACATTCTGGAAGATATCGTGCTGACCCTGACACTGTTTGAGGA
CAGAGAGATGATCGAGGAACGGCTGAAAACCTATGCCCACCTGTTCG
ACGACAAAGTGATGAAGCAGCTGAAGCGGCGGAGATACACCGGCTGG
GGCAGGCTGAGCCGGAAGCTGATCAACGGCATCCGGGACAAGCAGTC
CGGCAAGACAATCCTGGATTTCCTGAAGTCCGACGGCTTCGCCAACA
GAAACTTCATGCAGCTGATCCACGACGACAGCCTGACCTTTAAAGAG
GACATCCAGAAAGCCCAGGTGTCCGGCCAGGGCGATAGCCTGCACGA
GCACATTGCCAATCTGGCCGGCAGCCCCGCCATTAAGAAGGGCATCC
TGCAGACAGTGAAGGTGGTGGACGAGCTCGTGAAAGTGATGGGCCGG
CACAAGCCCGAGAACATCGTGATCGAAATGGCCAGAGAGAACCAGAC
CACCCAGAAGGGACAGAAGAACAGCCGCGAGAGAATGAAGCGGATCG
AAGAGGGCATCAAAGAGCTGGGCAGCCAGATCCTGAAAGAACACCCC
GTGGAAAACACCCAGCTGCAGAACGAGAAGCTGTACCTGTACTACCT
GCAGAATGGGCGGGATATGTACGTGGACCAGGAACTGGACATCAACC
GGCTGTCCGACTACGATGTGGACCATATCGTGCCTCAGAGCTTTCTG
AAGGACGACTCCATCGACAACAAGGTGCTGACCAGAAGCGACAAGAA
CCGGGGCAAGAGCGACAACGTGCCCTCCGAAGAGGTCGTGAAGAAGA
TGAAGAACTACTGGCGGCAGCTGCTGAACGCCAAGCTGATTACCCAG
AGAAAGTTCGACAATCTGACCAAGGCCGAGAGAGGCGGCCTGAGCGA
ACTGGATAAGGCCGGCTTCATCAAGAGACAGCTGGTGGAAACCCGGC
AGATCACAAAGCACGTGGCACAGATCCTGGACTCCCGGATGAACACT
AAGTACGACGAGAATGACAAGCTGATCCGGGAAGTGAAAGTGATCAC
CCTGAAGTCCAAGCTGGTGTCCGATTTCCGGAAGGATTTCCAGTTTT
ACAAAGTGCGCGAGATCAACAACTACCACCACGCCCACGACGCCTAC
CTGAACGCCGTCGTGGGAACCGCCCTGATCAAAAAGTACCCTAAGCT
GGAAAGCGAGTTCGTGTACGGCGACTACAAGGTGTACGACGTGCGGA
AGATGATCGCCAAGAGCGAGCAGGAAATCGGCAAGGCTACCGCCAAG
TACTTCTTCTACAGCAACATCATGAACTTTTTCAAGACCGAGATTAC
CCTGGCCAACGGCGAGATCCGGAAGCGGCCTCTGATCGAGACAAACG
GCGAAACCGGGGAGATCGTGTGGGATAAGGGCCGGGATTTTGCCACC
GTGCGGAAAGTGCTGAGCATGCCCCAAGTGAATATCGTGAAAAAGAC
CGAGGTGCAGACAGGCGGCTTCAGCAAAGAGTCTATCCTGCCCAAGA
GGAACAGCGATAAGCTGATCGCCAGAAAGAAGGACTGGGACCCTAAG
AAGTACGGCGGCTTCGACAGCCCCACCGTGGCCTATTCTGTGCTGGT
GGTGGCCAAAGTGGAAAAGGGCAAGTCCAAGAAACTGAAGAGTGTGA
AAGAGCTGCTGGGGATCACCATCATGGAAAGAAGCAGCTTCGAGAAG
AATCCCATCGACTTTCTGGAAGCCAAGGGCTACAAAGAAGTGAAAAA
GGACCTGATCATCAAGCTGCCTAAGTACTCCCTGTTCGAGCTGGAAA
ACGGCCGGAAGAGAATGCTGGCCTCTGCCGGCGAACTGCAGAAGGGA
AACGAACTGGCCCTGCCCTCCAAATATGTGAACTTCCTGTACCTGGC
CAGCCACTATGAGAAGCTGAAGGGCTCCCCCGAGGATAATGAGCAGA
AACAGCTGTTTGTGGAACAGCACAAGCACTACCTGGACGAGATCATC
GAGCAGATCAGCGAGTTCTCCAAGAGAGTGATCCTGGCCGACGCTAA
TCTGGACAAAGTGCTGTCCGCCTACAACAAGCACCGGGATAAGCCCA
TCAGAGAGCAGGCCGAGAATATCATCCACCTGTTTACCCTGACCAAT
CTGGGAGCCCCTGCCGCCTTCAAGTACTTTGACACCACCATCGACCG
GAAGAGGTACACCAGCACCAAAGAGGTGCTGGACGCCACCCTGATCC
ACCAGAGCATCACCGGCCTGTACGAGACACGGATCGACCTGTCTCAG
CTGGGAGGCGACGGATCCCCCAAGAAGAAGAGGAAAGTCTCGAGCGA
CTACAAAGACCATGACGGTGATTATAAAGATCATGACATCGATTACA
AGGATGACGATGACAAGGCTGCAGGATGA
Human codon optimized Streptococcus pyogenes
Cas9 (spCas9) Bipartite (BP) NLS
(SEQ ID NO: 179)
ATGGACAAGAAGTACAGCATCGGCCTGGACATCGGCACCAACTCTGT
GGGCTGGGCCGTGATCACCGACGAGTACAAGGTGCCCAGCAAGAAAT
TCAAGGTGCTGGGCAACACCGACCGGCACAGCATCAAGAAGAACCTG
ATCGGAGCCCTGCTGTTCGACAGCGGCGAAACAGCCGAGGCCACCCG
GCTGAAGAGAACCGCCAGAAGAAGATACACCAGACGGAAGAACCGGA
TCTGCTATCTGCAAGAGATCTTCAGCAACGAGATGGCCAAGGTGGAC
GACAGCTTCTTCCACAGACTGGAAGAGTCCTTCCTGGTGGAAGAGGA
TAAGAAGCACGAGCGGCACCCCATCTTCGGCAACATCGTGGACGAGG
TGGCCTACCACGAGAAGTACCCCACCATCTACCACCTGAGAAAGAAA
CTGGTGGACAGCACCGACAAGGCCGACCTGCGGCTGATCTATCTGGC
CCTGGCCCACATGATCAAGTTCCGGGGCCACTTCCTGATCGAGGGCG
ACCTGAACCCCGACAACAGCGACGTGGACAAGCTGTTCATCCAGCTG
GTGCAGACCTACAACCAGCTGTTCGAGGAAAACCCCATCAACGCCAG
CGGCGTGGACGCCAAGGCCATCCTGTCTGCCAGACTGAGCAAGAGCA
GACGGCTGGAAAATCTGATCGCCCAGCTGCCCGGCGAGAAGAAGAAT
GGCCTGTTCGGAAACCTGATTGCCCTGAGCCTGGGCCTGACCCCCAA
CTTCAAGAGCAACTTCGACCTGGCCGAGGATGCCAAACTGCAGCTGA
GCAAGGACACCTACGACGACGACCTGGACAACCTGCTGGCCCAGATC
GGCGACCAGTACGCCGACCTGTTTCTGGCCGCCAAGAACCTGTCCGA
CGCCATCCTGCTGAGCGACATCCTGAGAGTGAACACCGAGATCACCA
AGGCCCCCCTGAGCGCCTCTATGATCAAGAGATACGACGAGCACCAC
CAGGACCTGACCCTGCTGAAAGCTCTCGTGCGGCAGCAGCTGCCTGA
GAAGTACAAAGAGATTTTCTTCGACCAGAGCAAGAACGGCTACGCCG
GCTACATTGACGGCGGAGCCAGCCAGGAAGAGTTCTACAAGTTCATC
AAGCCCATCCTGGAAAAGATGGACGGCACCGAGGAACTGCTCGTGAA
GCTGAACAGAGAGGACCTGCTGCGGAAGCAGCGGACCTTCGACAACG
GCAGCATCCCCCACCAGATCCACCTGGGAGAGCTGCACGCCATTCTG
CGGCGGCAGGAAGATTTTTACCCATTCCTGAAGGACAACCGGGAAAA
GATCGAGAAGATCCTGACCTTCCGCATCCCCTACTACGTGGGCCCTC
TGGCCAGGGGAAACAGCAGATTCGCCTGGATGACCAGAAAGAGCGAG
GAAACCATCACCCCCTGGAACTTCGAGGAAGTGGTGGACAAGGGCGC
TTCCGCCCAGAGCTTCATCGAGCGGATGACCAACTTCGATAAGAACC
TGCCCAACGAGAAGGTGCTGCCCAAGCACAGCCTGCTGTACGAGTAC
TTCACCGTGTATAACGAGCTGACCAAAGTGAAATACGTGACCGAGGG
AATGAGAAAGCCCGCCTTCCTGAGCGGCGAGCAGAAAAAGGCCATCG
TGGACCTGCTGTTCAAGACCAACCGGAAAGTGACCGTGAAGCAGCTG
AAAGAGGACTACTTCAAGAAAATCGAGTGCTTCGACTCCGTGGAAAT
CTCCGGCGTGGAAGATCGGTTCAACGCCTCCCTGGGCACATACCACG
ATCTGCTGAAAATTATCAAGGACAAGGACTTCCTGGACAATGAGGAA
AACGAGGACATTCTGGAAGATATCGTGCTGACCCTGACACTGTTTGA
GGACAGAGAGATGATCGAGGAACGGCTGAAAACCTATGCCCACCTGT
TCGACGACAAAGTGATGAAGCAGCTGAAGCGGCGGAGATACACCGGC
TGGGGCAGGCTGAGCCGGAAGCTGATCAACGGCATCCGGGACAAGCA
GTCCGGCAAGACAATCCTGGATTTCCTGAAGTCCGACGGCTTCGCCA
ACAGAAACTTCATGCAGCTGATCCACGACGACAGCCTGACCTTTAAA
GAGGACATCCAGAAAGCCCAGGTGTCCGGCCAGGGCGATAGCCTGCA
CGAGCACATTGCCAATCTGGCCGGCAGCCCCGCCATTAAGAAGGGCA
TCCTGCAGACAGTGAAGGTGGTGGACGAGCTCGTGAAAGTGATGGGC
CGGCACAAGCCCGAGAACATCGTGATCGAAATGGCCAGAGAGAACCA
GACCACCCAGAAGGGACAGAAGAACAGCCGCGAGAGAATGAAGCGGA
TCGAAGAGGGCATCAAAGAGCTGGGCAGCCAGATCCTGAAAGAACAC
CCCGTGGAAAACACCCAGCTGCAGAACGAGAAGCTGTACCTGTACTA
CCTGCAGAATGGGCGGGATATGTACGTGGACCAGGAACTGGACATCA
ACCGGCTGTCCGACTACGATGTGGACCATATCGTGCCTCAGAGCTTT
CTGAAGGACGACTCCATCGACAACAAGGTGCTGACCAGAAGCGACAA
GAACCGGGGCAAGAGCGACAACGTGCCCTCCGAAGAGGTCGTGAAGA
AGATGAAGAACTACTGGCGGCAGCTGCTGAACGCCAAGCTGATTACC
CAGAGAAAGTTCGACAATCTGACCAAGGCCGAGAGAGGCGGCCTGAG
CGAACTGGATAAGGCCGGCTTCATCAAGAGACAGCTGGTGGAAACCC
GGCAGATCACAAAGCACGTGGCACAGATCCTGGACTCCCGGATGAAC
ACTAAGTACGACGAGAATGACAAGCTGATCCGGGAAGTGAAAGTGAT
CACCCTGAAGTCCAAGCTGGTGTCCGATTTCCGGAAGGATTTCCAGT
TTTACAAAGTGCGCGAGATCAACAACTACCACCACGCCCACGACGCC
TACCTGAACGCCGTCGTGGGAACCGCCCTGATCAAAAAGTACCCTAA
GCTGGAAAGCGAGTTCGTGTACGGCGACTACAAGGTGTACGACGTGC
GGAAGATGATCGCCAAGAGCGAGCAGGAAATCGGCAAGGCTACCGCC
AAGTACTTCTTCTACAGCAACATCATGAACTTTTTCAAGACCGAGAT
TACCCTGGCCAACGGCGAGATCCGGAAGCGGCCTCTGATCGAGACAA
ACGGCGAAACCGGGGAGATCGTGTGGGATAAGGGCCGGGATTTTGCC
ACCGTGCGGAAAGTGCTGAGCATGCCCCAAGTGAATATCGTGAAAAA
GACCGAGGTGCAGACAGGCGGCTTCAGCAAAGAGTCTATCCTGCCCA
AGAGGAACAGCGATAAGCTGATCGCCAGAAAGAAGGACTGGGACCCT
AAGAAGTACGGCGGCTTCGACAGCCCCACCGTGGCCTATTCTGTGCT
GGTGGTGGCCAAAGTGGAAAAGGGCAAGTCCAAGAAACTGAAGAGTG
TGAAAGAGCTGCTGGGGATCACCATCATGGAAAGAAGCAGCTTCGAG
AAGAATCCCATCGACTTTCTGGAAGCCAAGGGCTACAAAGAAGTGAA
AAAGGACCTGATCATCAAGCTGCCTAAGTACTCCCTGTTCGAGCTGG
AAAACGGCCGGAAGAGAATGCTGGCCTCTGCCGGCGAACTGCAGAAG
GGAAACGAACTGGCCCTGCCCTCCAAATATGTGAACTTCCTGTACCT
GGCCAGCCACTATGAGAAGCTGAAGGGCTCCCCCGAGGATAATGAGC
AGAAACAGCTGTTTGTGGAACAGCACAAGCACTACCTGGACGAGATC
ATCGAGCAGATCAGCGAGTTCTCCAAGAGAGTGATCCTGGCCGACGC
TAATCTGGACAAAGTGCTGTCCGCCTACAACAAGCACCGGGATAAGC
CCATCAGAGAGCAGGCCGAGAATATCATCCACCTGTTTACCCTGACC
AATCTGGGAGCCCCTGCCGCCTTCAAGTACTTTGACACCACCATCGA
CCGGAAGAGGTACACCAGCACCAAAGAGGTGCTGGACGCCACCCTGA
TCCACCAGAGCATCACCGGCCTGTACGAGACACGGATCGACCTGTCT
CAGCTGGGAGGCGACGGATCCGGCGGAGGCGGAAGCGGGAAAAGAAC
CGCCGACGGCAGCGAATTCGAGCCCAAGAAGAAGAGGAAAGTCTCGA
GCGGAGGCGACTACAAAGACCATGACGGTGATTATAAAGATCATGAC
ATCGATTACAAGGATGACGATGACAAGTGA
Human codon optimized Streptococcus pyogenes
Cas9 (spCas9) BE4
(SEQ ID NO: 180)
ATGAAACGGACAGCCGACGGAAGCGAGTTCGAGTCACCAAAGAAGAA
GCGGAAAGTCTCCTCAGAGACTGGGCCTGTCGCCGTCGATCCAACCC
TGCGCCGCCGGATTGAACCTCACGAGTTTGAAGTGTTCTTTGACCCC
CGGGAGCTGAGAAAGGAGACATGCCTGCTGTACGAGATCAACTGGGG
AGGCAGGCACTCCATCTGGAGGCACACCTCTCAGAACACAAATAAGC
ACGTGGAGGTGAACTTCATCGAGAAGTTTACCACAGAGCGGTACTTC
TGCCCCAATACCAGATGTAGCATCACATGGTTTCTGAGCTGGTCCCC
TTGCGGAGAGTGTAGCAGGGCCATCACCGAGTTCCTGTCCAGATATC
CACACGTGACACTGTTTATCTACATCGCCAGGCTGTATCACCACGCA
GACCCAAGGAATAGGCAGGGCCTGCGCGATCTGATCAGCTCCGGCGT
GACCATCCAGATCATGACAGAGCAGGAGTCCGGCTACTGCTGGCGGA
ACTTCGTGAATTATTCTCCTAGCAACGAGGCCCACTGGCCTAGGTAC
CCACACCTGTGGGTGCGCCTGTACGTGCTGGAGCTGTATTGCATCAT
CCTGGGCCTGCCCCCTTGTCTGAATATCCTGCGGAGAAAGCAGCCCC
AGCTGACCTTCTTTACAATCGCCCTGCAGTCTTGTCACTATCAGAGG
CTGCCACCCCACATCCTGTGGGCCACAGGCCTGAAGTCTGGAGGATC
TAGCGGAGGATCCTCTGGCAGCGAGACACCAGGAACAAGCGAGTCAG
CAACACCAGAGAGCAGTGGCGGCAGCAGCGGCGGCAGCGACAAGAAG
TACAGCATCGGCCTGGACATCGGCACCAACTCTGTGGGCTGGGCCGT
GATCACCGACGAGTACAAGGTGCCCAGCAAGAAATTCAAGGTGCTGG
GCAACACCGACCGGCACAGCATCAAGAAGAACCTGATCGGAGCCCTG
CTGTTCGACAGCGGCGAAACAGCCGAGGCCACCCGGCTGAAGAGAAC
CGCCAGAAGAAGATACACCAGACGGAAGAACCGGATCTGCTATCTGC
AAGAGATCTTCAGCAACGAGATGGCCAAGGTGGACGACAGCTTCTTC
CACAGACTGGAAGAGTCCTTCCTGGTGGAAGAGGATAAGAAGCACGA
GCGGCACCCCATCTTCGGCAACATCGTGGACGAGGTGGCCTACCACG
AGAAGTACCCCACCATCTACCACCTGAGAAAGAAACTGGTGGACAGC
ACCGACAAGGCCGACCTGCGGCTGATCTATCTGGCCCTGGCCCACAT
GATCAAGTTCCGGGGCCACTTCCTGATCGAGGGCGACCTGAACCCCG
ACAACAGCGACGTGGACAAGCTGTTCATCCAGCTGGTGCAGACCTAC
AACCAGCTGTTCGAGGAAAACCCCATCAACGCCAGCGGCGTGGACGC
CAAGGCCATCCTGTCTGCCAGACTGAGCAAGAGCAGACGGCTGGAAA
ATCTGATCGCCCAGCTGCCCGGCGAGAAGAAGAATGGCCTGTTCGGA
AACCTGATTGCCCTGAGCCTGGGCCTGACCCCCAACTTCAAGAGCAA
CTTCGACCTGGCCGAGGATGCCAAACTGCAGCTGAGCAAGGACACCT
ACGACGACGACCTGGACAACCTGCTGGCCCAGATCGGCGACCAGTAC
GCCGACCTGTTTCTGGCCGCCAAGAACCTGTCCGACGCCATCCTGCT
GAGCGACATCCTGAGAGTGAACACCGAGATCACCAAGGCCCCCCTGA
GCGCCTCTATGATCAAGAGATACGACGAGCACCACCAGGACCTGACC
CTGCTGAAAGCTCTCGTGCGGCAGCAGCTGCCTGAGAAGTACAAAGA
GATTTTCTTCGACCAGAGCAAGAACGGCTACGCCGGCTACATTGACG
GCGGAGCCAGCCAGGAAGAGTTCTACAAGTTCATCAAGCCCATCCTG
GAAAAGATGGACGGCACCGAGGAACTGCTCGTGAAGCTGAACAGAGA
GGACCTGCTGCGGAAGCAGCGGACCTTCGACAACGGCAGCATCCCCC
ACCAGATCCACCTGGGAGAGCTGCACGCCATTCTGCGGCGGCAGGAA
GATTTTTACCCATTCCTGAAGGACAACCGGGAAAAGATCGAGAAGAT
CCTGACCTTCCGCATCCCCTACTACGTGGGCCCTCTGGCCAGGGGAA
ACAGCAGATTCGCCTGGATGACCAGAAAGAGCGAGGAAACCATCACC
CCCTGGAACTTCGAGGAAGTGGTGGACAAGGGCGCTTCCGCCCAGAG
CTTCATCGAGCGGATGACCAACTTCGATAAGAACCTGCCCAACGAGA
AGGTGCTGCCCAAGCACAGCCTGCTGTACGAGTACTTCACCGTGTAT
AACGAGCTGACCAAAGTGAAATACGTGACCGAGGGAATGAGAAAGCC
CGCCTTCCTGAGCGGCGAGCAGAAAAAGGCCATCGTGGACCTGCTGT
TCAAGACCAACCGGAAAGTGACCGTGAAGCAGCTGAAAGAGGACTAC
TTCAAGAAAATCGAGTGCTTCGACTCCGTGGAAATCTCCGGCGTGGA
AGATCGGTTCAACGCCTCCCTGGGCACATACCACGATCTGCTGAAAA
TTATCAAGGACAAGGACTTCCTGGACAATGAGGAAAACGAGGACATT
CTGGAAGATATCGTGCTGACCCTGACACTGTTTGAGGACAGAGAGAT
GATCGAGGAACGGCTGAAAACCTATGCCCACCTGTTCGACGACAAAG
TGATGAAGCAGCTGAAGCGGCGGAGATACACCGGCTGGGGCAGGCTG
AGCCGGAAGCTGATCAACGGCATCCGGGACAAGCAGTCCGGCAAGAC
AATCCTGGATTTCCTGAAGTCCGACGGCTTCGCCAACAGAAACTTCA
TGCAGCTGATCCACGACGACAGCCTGACCTTTAAAGAGGACATCCAG
AAAGCCCAGGTGTCCGGCCAGGGCGATAGCCTGCACGAGCACATTGC
CAATCTGGCCGGCAGCCCCGCCATTAAGAAGGGCATCCTGCAGACAG
TGAAGGTGGTGGACGAGCTCGTGAAAGTGATGGGCCGGCACAAGCCC
GAGAACATCGTGATCGAAATGGCCAGAGAGAACCAGACCACCCAGAA
GGGACAGAAGAACAGCCGCGAGAGAATGAAGCGGATCGAAGAGGGCA
TCAAAGAGCTGGGCAGCCAGATCCTGAAAGAACACCCCGTGGAAAAC
ACCCAGCTGCAGAACGAGAAGCTGTACCTGTACTACCTGCAGAATGG
GCGGGATATGTACGTGGACCAGGAACTGGACATCAACCGGCTGTCCG
ACTACGATGTGGACCATATCGTGCCTCAGAGCTTTCTGAAGGACGAC
TCCATCGACAACAAGGTGCTGACCAGAAGCGACAAGAACCGGGGCAA
GAGCGACAACGTGCCCTCCGAAGAGGTCGTGAAGAAGATGAAGAACT
ACTGGCGGCAGCTGCTGAACGCCAAGCTGATTACCCAGAGAAAGTTC
GACAATCTGACCAAGGCCGAGAGAGGCGGCCTGAGCGAACTGGATAA
GGCCGGCTTCATCAAGAGACAGCTGGTGGAAACCCGGCAGATCACAA
AGCACGTGGCACAGATCCTGGACTCCCGGATGAACACTAAGTACGAC
GAGAATGACAAGCTGATCCGGGAAGTGAAAGTGATCACCCTGAAGTC
CAAGCTGGTGTCCGATTTCCGGAAGGATTTCCAGTTTTACAAAGTGC
GCGAGATCAACAACTACCACCACGCCCACGACGCCTACCTGAACGCC
GTCGTGGGAACCGCCCTGATCAAAAAGTACCCTAAGCTGGAAAGCGA
GTTCGTGTACGGCGACTACAAGGTGTACGACGTGCGGAAGATGATCG
CCAAGAGCGAGCAGGAAATCGGCAAGGCTACCGCCAAGTACTTCTTC
TACAGCAACATCATGAACTTTTTCAAGACCGAGATTACCCTGGCCAA
CGGCGAGATCCGGAAGCGGCCTCTGATCGAGACAAACGGCGAAACCG
GGGAGATCGTGTGGGATAAGGGCCGGGATTTTGCCACCGTGCGGAAA
GTGCTGAGCATGCCCCAAGTGAATATCGTGAAAAAGACCGAGGTGCA
GACAGGCGGCTTCAGCAAAGAGTCTATCCTGCCCAAGAGGAACAGCG
ATAAGCTGATCGCCAGAAAGAAGGACTGGGACCCTAAGAAGTACGGC
GGCTTCGACAGCCCCACCGTGGCCTATTCTGTGCTGGTGGTGGCCAA
AGTGGAAAAGGGCAAGTCCAAGAAACTGAAGAGTGTGAAAGAGCTGC
TGGGGATCACCATCATGGAAAGAAGCAGCTTCGAGAAGAATCCCATC
GACTTTCTGGAAGCCAAGGGCTACAAAGAAGTGAAAAAGGACCTGAT
CATCAAGCTGCCTAAGTACTCCCTGTTCGAGCTGGAAAACGGCCGGA
AGAGAATGCTGGCCTCTGCCGGCGAACTGCAGAAGGGAAACGAACTG
GCCCTGCCCTCCAAATATGTGAACTTCCTGTACCTGGCCAGCCACTA
TGAGAAGCTGAAGGGCTCCCCCGAGGATAATGAGCAGAAACAGCTGT
TTGTGGAACAGCACAAGCACTACCTGGACGAGATCATCGAGCAGATC
AGCGAGTTCTCCAAGAGAGTGATCCTGGCCGACGCTAATCTGGACAA
AGTGCTGTCCGCCTACAACAAGCACCGGGATAAGCCCATCAGAGAGC
AGGCCGAGAATATCATCCACCTGTTTACCCTGACCAATCTGGGAGCC
CCTGCCGCCTTCAAGTACTTTGACACCACCATCGACCGGAAGAGGTA
CACCAGCACCAAAGAGGTGCTGGACGCCACCCTGATCCACCAGAGCA
TCACCGGCCTGTACGAGACACGGATCGACCTGTCTCAGCTGGGAGGT
GACAGCGGCGGGAGCGGCGGGAGCGGGGGGAGCACTAATCTGAGCGA
CATCATTGAGAAGGAGACTGGGAAACAGCTGGTCATTCAGGAGTCCA
TCCTGATGCTGCCTGAGGAGGTGGAGGAAGTGATCGGCAACAAGCCA
GAGTCTGACATCCTGGTGCACACCGCCTACGACGAGTCCACAGATGA
GAATGTGATGCTGCTGACCTCTGACGCCCCCGAGTATAAGCCTTGGG
CCCTGGTCATCCAGGATTCTAACGGCGAGAATAAGATCAAGATGCTG
AGCGGAGGATCCGGAGGATCTGGAGGCAGCACCAACCTGTCTGACAT
CATCGAGAAGGAGACAGGCAAGCAGCTGGTCATCCAGGAGAGCATCC
TGATGCTGCCCGAAGAAGTCGAAGAAGTGATCGGAAACAAGCCTGAG
AGCGATATCCTGGTCCATACCGCCTACGACGAGAGTACCGACGAAAA
TGTGATGCTGCTGACATCCGACGCCCCAGAGTATAAGCCCTGGGCTC
TGGTCATCCAGGATTCCAACGGAGAGAACAAAATCAAAATGCTGTCT
GGCGGCTCAAAAAGAACCGCCGACGGCAGCGAATTCGAGCCCAAGAA
GAAGAGGAAAGTCTAA
Human codon optimized Streptococcus pyogenes
Cas9 (spCas9) ABE
(SEQ ID NO: 181)
ATGAAACGGACAGCCGACGGAAGCGAGTTCGAGTCACCAAAGAAGAAGC
GGAAAGTCTCTGAAGTCGAGTTTAGCCACGAGTATTGGATGAGGCACG
CACTGACCCTGGCAAAGCGAGCATGGGATGAAAGAGAAGTCCCCGTG
GGCGCCGTGCTGGTGCACAACAATAGAGTGATCGGAGAGGGATGGAA
CAGGCCAATCGGCCGCCACGACCCTACCGCACACGCAGAGATCATGG
CACTGAGGCAGGGAGGCCTGGTCATGCAGAATTACCGCCTGATCGAT
GCCACCCTGTATGTGACACTGGAGCCATGCGTGATGTGCGCAGGAGC
AATGATCCACAGCAGGATCGGAAGAGTGGTGTTCGGAGCACGGGACG
CCAAGACCGGCGCAGCAGGCTCCCTGATGGATGTGCTGCACCACCCC
GGCATGAACCACCGGGTGGAGATCACAGAGGGAATCCTGGCAGACGA
GTGCGCCGCCCTGCTGAGCGATTTCTTTAGAATGCGGAGACAGGAGA
TCAAGGCCCAGAAGAAGGCACAGAGCTCCACCGACTCTGGAGGATCT
AGCGGAGGATCCTCTGGAAGCGAGACACCAGGCACAAGCGAGTCCGC
CACACCAGAGAGCTCCGGCGGCTCCTCCGGAGGATCCTCTGAGGTGG
AGTTTTCCCACGAGTACTGGATGAGACATGCCCTGACCCTGGCCAAG
AGGGCACGCGATGAGAGGGAGGTGCCTGTGGGAGCCGTGCTGGTGCT
GAACAATAGAGTGATCGGCGAGGGCTGGAACAGAGCCATCGGCCTGC
ACGACCCAACAGCCCATGCCGAAATTATGGCCCTGAGACAGGGCGGC
CTGGTCATGCAGAACTACAGACTGATTGACGCCACCCTGTACGTGAC
ATTCGAGCCTTGCGTGATGTGCGCCGGCGCCATGATCCACTCTAGGA
TCGGCCGCGTGGTGTTTGGCGTGAGGAACGCAAAAACCGGCGCCGCA
GGCTCCCTGATGGACGTGCTGCACTACCCCGGCATGAATCACCGCGT
CGAAATTACCGAGGGAATCCTGGCAGATGAATGTGCCGCCCTGCTGT
GCTATTTCTTTCGGATGCCTAGACAGGTGTTCAATGCTCAGAAGAAG
GCCCAGAGCTCCACCGACTCCGGAGGATCTAGCGGAGGCTCCTCTGG
CTCTGAGACACCTGGCACAAGCGAGAGCGCAACACCTGAAAGCAGCG
GGGGCAGCAGCGGGGGGTCAGACAAGAAGTACAGCATCGGCCTGGCC
ATCGGCACCAACTCTGTGGGCTGGGCCGTGATCACCGACGAGTACAA
GGTGCCCAGCAAGAAATTCAAGGTGCTGGGCAACACCGACCGGCACA
GCATCAAGAAGAACCTGATCGGAGCCCTGCTGTTCGACAGCGGCGAA
ACAGCCGAGGCCACCCGGCTGAAGAGAACCGCCAGAAGAAGATACAC
CAGACGGAAGAACCGGATCTGCTATCTGCAAGAGATCTTCAGCAACG
AGATGGCCAAGGTGGACGACAGCTTCTTCCACAGACTGGAAGAGTCC
TTCCTGGTGGAAGAGGATAAGAAGCACGAGCGGCACCCCATCTTCGG
CAACATCGTGGACGAGGTGGCCTACCACGAGAAGTACCCCACCATCT
ACCACCTGAGAAAGAAACTGGTGGACAGCACCGACAAGGCCGACCTG
CGGCTGATCTATCTGGCCCTGGCCCACATGATCAAGTTCCGGGGCCA
CTTCCTGATCGAGGGCGACCTGAACCCCGACAACAGCGACGTGGACA
AGCTGTTCATCCAGCTGGTGCAGACCTACAACCAGCTGTTCGAGGAA
AACCCCATCAACGCCAGCGGCGTGGACGCCAAGGCCATCCTGTCTGC
CAGACTGAGCAAGAGCAGACGGCTGGAAAATCTGATCGCCCAGCTGC
CCGGCGAGAAGAAGAATGGCCTGTTCGGAAACCTGATTGCCCTGAGC
CTGGGCCTGACCCCCAACTTCAAGAGCAACTTCGACCTGGCCGAGGA
TGCCAAACTGCAGCTGAGCAAGGACACCTACGACGACGACCTGGACA
ACCTGCTGGCCCAGATCGGCGACCAGTACGCCGACCTGTTTCTGGCC
GCCAAGAACCTGTCCGACGCCATCCTGCTGAGCGACATCCTGAGAGT
GAACACCGAGATCACCAAGGCCCCCCTGAGCGCCTCTATGATCAAGA
GATACGACGAGCACCACCAGGACCTGACCCTGCTGAAAGCTCTCGTG
CGGCAGCAGCTGCCTGAGAAGTACAAAGAGATTTTCTTCGACCAGAG
CAAGAACGGCTACGCCGGCTACATTGACGGCGGAGCCAGCCAGGAAG
AGTTCTACAAGTTCATCAAGCCCATCCTGGAAAAGATGGACGGCACC
GAGGAACTGCTCGTGAAGCTGAACAGAGAGGACCTGCTGCGGAAGCA
GCGGACCTTCGACAACGGCAGCATCCCCCACCAGATCCACCTGGGAG
AGCTGCACGCCATTCTGCGGCGGCAGGAAGATTTTTACCCATTCCTG
AAGGACAACCGGGAAAAGATCGAGAAGATCCTGACCTTCCGCATCCC
CTACTACGTGGGCCCTCTGGCCAGGGGAAACAGCAGATTCGCCTGGA
TGACCAGAAAGAGCGAGGAAACCATCACCCCCTGGAACTTCGAGGAA
GTGGTGGACAAGGGCGCTTCCGCCCAGAGCTTCATCGAGCGGATGAC
CAACTTCGATAAGAACCTGCCCAACGAGAAGGTGCTGCCCAAGCACA
GCCTGCTGTACGAGTACTTCACCGTGTATAACGAGCTGACCAAAGTG
AAATACGTGACCGAGGGAATGAGAAAGCCCGCCTTCCTGAGCGGCGA
GCAGAAAAAGGCCATCGTGGACCTGCTGTTCAAGACCAACCGGAAAG
TGACCGTGAAGCAGCTGAAAGAGGACTACTTCAAGAAAATCGAGTGC
TTCGACTCCGTGGAAATCTCCGGCGTGGAAGATCGGTTCAACGCCTC
CCTGGGCACATACCACGATCTGCTGAAAATTATCAAGGACAAGGACT
TCCTGGACAATGAGGAAAACGAGGACATTCTGGAAGATATCGTGCTG
ACCCTGACACTGTTTGAGGACAGAGAGATGATCGAGGAACGGCTGAA
AACCTATGCCCACCTGTTCGACGACAAAGTGATGAAGCAGCTGAAGC
GGCGGAGATACACCGGCTGGGGCAGGCTGAGCCGGAAGCTGATCAAC
GGCATCCGGGACAAGCAGTCCGGCAAGACAATCCTGGATTTCCTGAA
GTCCGACGGCTTCGCCAACAGAAACTTCATGCAGCTGATCCACGACG
ACAGCCTGACCTTTAAAGAGGACATCCAGAAAGCCCAGGTGTCCGGC
CAGGGCGATAGCCTGCACGAGCACATTGCCAATCTGGCCGGCAGCCC
CGCCATTAAGAAGGGCATCCTGCAGACAGTGAAGGTGGTGGACGAGC
TCGTGAAAGTGATGGGCCGGCACAAGCCCGAGAACATCGTGATCGAA
ATGGCCAGAGAGAACCAGACCACCCAGAAGGGACAGAAGAACAGCCG
CGAGAGAATGAAGCGGATCGAAGAGGGCATCAAAGAGCTGGGCAGCC
AGATCCTGAAAGAACACCCCGTGGAAAACACCCAGCTGCAGAACGAG
AAGCTGTACCTGTACTACCTGCAGAATGGGCGGGATATGTACGTGGA
CCAGGAACTGGACATCAACCGGCTGTCCGACTACGATGTGGACCATA
TCGTGCCTCAGAGCTTTCTGAAGGACGACTCCATCGACAACAAGGTG
CTGACCAGAAGCGACAAGAACCGGGGCAAGAGCGACAACGTGCCCTC
CGAAGAGGTCGTGAAGAAGATGAAGAACTACTGGCGGCAGCTGCTGA
ACGCCAAGCTGATTACCCAGAGAAAGTTCGACAATCTGACCAAGGCC
GAGAGAGGCGGCCTGAGCGAACTGGATAAGGCCGGCTTCATCAAGAG
ACAGCTGGTGGAAACCCGGCAGATCACAAAGCACGTGGCACAGATCC
TGGACTCCCGGATGAACACTAAGTACGACGAGAATGACAAGCTGATC
CGGGAAGTGAAAGTGATCACCCTGAAGTCCAAGCTGGTGTCCGATTT
CCGGAAGGATTTCCAGTTTTACAAAGTGCGCGAGATCAACAACTACC
ACCACGCCCACGACGCCTACCTGAACGCCGTCGTGGGAACCGCCCTG
ATCAAAAAGTACCCTAAGCTGGAAAGCGAGTTCGTGTACGGCGACTA
CAAGGTGTACGACGTGCGGAAGATGATCGCCAAGAGCGAGCAGGAAA
TCGGCAAGGCTACCGCCAAGTACTTCTTCTACAGCAACATCATGAAC
TTTTTCAAGACCGAGATTACCCTGGCCAACGGCGAGATCCGGAAGCG
GCCTCTGATCGAGACAAACGGCGAAACCGGGGAGATCGTGTGGGATA
AGGGCCGGGATTTTGCCACCGTGCGGAAAGTGCTGAGCATGCCCCAA
GTGAATATCGTGAAAAAGACCGAGGTGCAGACAGGCGGCTTCAGCAA
AGAGTCTATCCTGCCCAAGAGGAACAGCGATAAGCTGATCGCCAGAA
AGAAGGACTGGGACCCTAAGAAGTACGGCGGCTTCGACAGCCCCACC
GTGGCCTATTCTGTGCTGGTGGTGGCCAAAGTGGAAAAGGGCAAGTC
CAAGAAACTGAAGAGTGTGAAAGAGCTGCTGGGGATCACCATCATGG
AAAGAAGCAGCTTCGAGAAGAATCCCATCGACTTTCTGGAAGCCAAG
GGCTACAAAGAAGTGAAAAAGGACCTGATCATCAAGCTGCCTAAGTA
CTCCCTGTTCGAGCTGGAAAACGGCCGGAAGAGAATGCTGGCCTCTG
CCGGCGAACTGCAGAAGGGAAACGAACTGGCCCTGCCCTCCAAATAT
GTGAACTTCCTGTACCTGGCCAGCCACTATGAGAAGCTGAAGGGCTC
CCCCGAGGATAATGAGCAGAAACAGCTGTTTGTGGAACAGCACAAGC
ACTACCTGGACGAGATCATCGAGCAGATCAGCGAGTTCTCCAAGAGA
GTGATCCTGGCCGACGCTAATCTGGACAAAGTGCTGTCCGCCTACAA
CAAGCACCGGGATAAGCCCATCAGAGAGCAGGCCGAGAATATCATCC
ACCTGTTTACCCTGACCAATCTGGGAGCCCCTGCCGCCTTCAAGTAC
TTTGACACCACCATCGACCGGAAGAGGTACACCAGCACCAAAGAGGT
GCTGGACGCCACCCTGATCCACCAGAGCATCACCGGCCTGTACGAGA
CACGGATCGACCTGTCTCAGCTGGGAGGTGACTCTGGCGGCTCAAAA
AGAACCGCCGACGGCAGCGAATTCGAGCCCAAGAAGAAGAGGAAAGT
CTAA
Human codon optimized VSVG
(SEQ ID NO: 182)
ATGAAATGTCTGCTGTACCTGGCTTTCCTGTTCATCGGCGTGAACTG
CAAGTTCACCATCGTGTTCCCTCACAACCAGAAGGGCAACTGGAAAA
ATGTGCCTAGCAACTACCACTACTGTCCTAGCTCTAGCGACCTTAAT
TGGCATAACGACCTGATCGGCACAGCCCTGCAGGTGAAGATGCCTAA
GAGCCACAAGGCCATCCAGGCCGACGGATGGATGTGCCACGCCAGCA
AGTGGGTCACAACCTGTGACTTCAGATGGTACGGCCCTAAATACATT
ACCCACTCTATCAGAAGCTTCACCCCTTCTGTGGAACAATGTAAAGA
GTCCATTGAGCAGACAAAGCAGGGCACCTGGCTGAACCCCGGCTTCC
CCCCCCAGAGCTGCGGCTACGCCACCGTTACCGATGCCGAGGCCGTG
ATCGTGCAGGTGACACCTCACCACGTGCTGGTCGATGAGTACACCGG
CGAATGGGTGGACAGCCAATTTATCAACGGCAAATGCAGCAATTACA
TCTGCCCCACCGTGCACAACAGCACCACCTGGCACAGCGATTACAAG
GTGAAAGGCCTGTGCGACAGCAACCTGATCTCTATGGACATCACCTT
CTTCAGCGAGGACGGCGAGCTGTCTAGTCTGGGCAAGGAAGGCACAG
GTTTTCGGAGCAACTACTTCGCCTACGAGACTGGCGGCAAGGCCTGC
AAGATGCAGTACTGCAAGCACTGGGGCGTTAGACTGCCTTCAGGCGT
GTGGTTCGAGATGGCCGATAAGGACCTGTTCGCCGCTGCCAGATTCC
CAGAGTGCCCTGAGGGCAGCTCCATCAGCGCCCCTTCCCAGACCTCC
GTGGATGTGTCCCTGATCCAGGACGTGGAAAGAATCCTGGACTACAG
CCTCTGTCAGGAGACATGGTCCAAAATCAGAGCCGGACTCCCCATTA
GCCCTGTGGACCTGAGCTACCTGGCCCCCAAGAATCCTGGAACCGGC
CCCGCCTTCACAATCATTAACGGCACCCTGAAATACTTCGAGACCAG
ATACATCCGGGTGGACATCGCCGCTCCTATCCTGTCAAGAATGGTGG
GCATGATTTCTGGCACAACAACAGAGAGGGAACTGTGGGACGACTGG
GCCCCTTACGAGGATGTGGAAATCGGCCCAAACGGCGTGCTGCGGAC
CAGCTCAGGCTATAAGTTCCCCCTGTACATGATCGGCCACGGCATGC
TGGATTCTGACCTGCACCTGAGCAGCAAGGCCCAGGTCTTTGAGCAC
CCTCATATCCAAGACGCCGCCAGCCAGCTGCCTGATGACGAGAGCCT
GTTTTTTGGAGATACAGGACTGAGCAAGAACCCCATCGAGCTGGTGG
AAGGCTGGTTTAGCAGCTGGAAGTCCAGCATAGCTAGCTTCTTCTTC
ATCATCGGCCTGATCATCGGACTGTTCCTGGTGCTGAGAGTGGGGAT
CCACCTGTGCATCAAGCTGAAGCACACCAAAAAGAGACAGATCTACA
CCGACATCGAGATGAACCGGCTGGGGAAGTGA

### LITERATURE CITED

1. Parseval, N. et al. Survey of human genes of retroviral origin: identification and transcriptome of the genes with coding capacity for complete envelope proteins. Journal of Virology 77, 10414-10422, (2003).
2. Okimoto, T. et al. VSV-G envelope glycoprotein forms complexes with plasmid DNA and MLV retrovirus-like particles in cell-free conditions and enhances DNA transfection. Molecular Therapy 4, 232-238, (2001).
3. Mangeot, P. et al. Protein transfer into human cells by VSV-G-induced nanovesicles. Molecular Therapy 19, 1656-1666, (2011).
4. Wagner, D. et al. High prevalence of Streptococcus pyogenes Cas9-reactive T cells within the adult human population. Nature Medicine 25, 242-248 (2019)
5. Kim, S. et al. CRISPR RNAs trigger innate immune responses in human cells. Genome Research 28, 1-7 (2018).
6. Charlesworth, C. et al. Identification of preexisting adaptive immunity to Cas9 proteins in humans. Nature Medicine 25, 249-254 (2019)
7. Ferdosi, S. et al. Multifunctional CRISPR-Cas9 with engineered immunosilenced human T cell epitopes. Nature Communications 10, Article number: 1842 (2019).
8. Wang, D. et al. Adenovirus-mediated somatic genome editing of Pten by CRISPR/Cas9 in mouse liver in spite of Cas9-specific immune responses. Human Gene Therapy 26, 432-442 (2015).
9. Devanabanda, M. et al. Immunotoxic effects of gold and silver nanoparticles: Inhibition of mitogen-induced proliferative responses and viability of human and murine lymphocytes in vitro. Journal of Immunotoxicology 13, 1547-6901 (2016).
10. Mout, R. et al. Direct cytosolic delivery of CRISPR/Cas9-ribonucleoprotein for efficient gene editing. ACS Nano 11, 2452-2458 (2017).
11. Yin, H. et al. structure-guided chemical modification of guide RNA enables potent non-viral in vivo genome editing. Nature Biotechnology 35, 1179-1187 (2017).
12. Qiao, J. et al. Cytosolic delivery of CRISPR/Cas9 ribonucleoproteins for genome editing using chitosan-coated red fluorescent protein. Chemical Communications 55, 4707-4710 (2019).
13. Li, L. et al. A rationally designed semiconducting polymer brush for NIR-II imaging guided light-triggered remote control of CRISPR/Cas9 genome editing. Advanced Materials 1901187, 1-9 (2019).
14. Gao, X. et al. Treatment of autosomal dominant hearing loss by in vivo delivery of genome editing agents. Nature 553, 217-221 (2018)
15. Lee, K. et al. Nanoparticle delivery of Cas9 ribonucleoprotein and donor DNA in vivo induces homology-directed DNA repair. Nature Biomedical Engineering 1, 889-901 (2017).
16. Staahl, B. et al. Efficient genome editing in the mouse brain by local delivery of engineered Cas9 ribonucleoprotein complexes. Nature Biotechnology 35, 431-433 (2017).
17. Zuris, J. et al. Cationic lipid-mediated delivery of proteins enables efficient protein-based genome editing in vitro and in vivo. Nature Biotechnology 33, 73-79 (2015).
18. Finn, J. et al. A single administration of CRISPR/Cas9 lipid nanoparticles achieves robust and persistent in vivo genome editing. Cell Reports 22, 2227-2235 (2018).
19. Wang, H. et al. Nonviral gene editing via CRISPR/Cas9 delivery by membrane-disruptive and endosomolytic helical polypeptide. PNAS 115, 4903-4908 (2018).
20. Del′Guidice, T. et al. Membrane permeabilizing amphiphilic peptide delivers recombinant transcription factor and CRISPR-Cas9/Cpf1 ribonucleoproteins in hard-to-modify cells. PLOS ONE 13, e0195558 (2018).
21. Colella, P. et al. Emerging Issues in AAV-Mediated In Vivo Gene Therapy. Molecular Therapy: Methods & Clinical Development 8, 87-104 (2018).
22. Naso, F. et al. Adeno-Associated Virus (AAV) as a Vector for Gene Therapy. BioDrugs 31, 317-334 (2017).
23. Handel, E. et al. Versatile and efficient genome editing in human cells by combining zinc-finger nucleases with adeno-associated viral vectors. Human Gene Therapy 23, 321-329 (2012).
24. Chadwick, A. et al. Reduced Blood Lipid Levels With In Vivo CRISPR-Cas9 Base Editing of ANGPTL3. Circulation 137, 975-977 (2018).
25. Schenkwein, D. et al. Production of HIV-1 Integrase Fusion Protein-Carrying Lentiviral Vectors for Gene Therapy and Protein Transduction. Human Gene Therapy 21, 589-602 (2010).
26. Cai, Y. et al. Targeted genome editing by lentiviral protein transduction of zinc-finger and TAL-effector nucleases. eLife 3, e01911 (2014).
27. Choi, J. et al. Lentivirus pre-packed with Cas9 protein for safer gene editing. Gene Therapy 23, 627-633 (2016).
28. Meyer, C. et al. Pseudotyping exosomes for enhanced protein delivery in mammalian cells. International Journal of Nanomedicine 12, 3153-3170 (2017).
29. Mangeot, P. et al. Genome editing in primary cells and in vivo using viral-derived Nanoblades loaded with Cas9-sgRNA ribonucleoproteins. Nature Communications 10, Article number: 45 (2019).
30. Lu, B. et al. Delivering SaCas9 mRNA by lentivirus-like bionanoparticles for transient expression and efficient genome editing. Nucleic Acids Research 47, e44 (2019).
31. Wang, Q. et al. ARMMs as a versatile platform for intracellular delivery of macromolecules. Nature Communications 9, 1-7 (2018).
32. Lainscek, D. et al. Delivery of an Artificial Transcription Regulator dCas9-VPR by Extracellular Vesicles for Therapeutic Gene Activation. ACS Synthetic Biology 7, 2715-2725(2018).
33. Fuchs, J. et al. First-in-Human Evaluation of the Safety and Immunogenicity of a Recombinant Vesicular Stomatitis Virus Human Immunodeficiency Virus-1 gag Vaccine (HVTN 090). Open Forum Infectious Diseases 2, 1-9, (2015).
34. Cong, L. et al. Multiplex Genome Engineering Using CRISPR/Cas Systems. Science 339, 819-823, (2013).
35. Ran, F. et al. In vivo genome editing using Staphylococcus aureus Cas9. Nature 520, 186-191, (2015).
36. Zetsche, B. et al. Cpf1 Is a Single RNA-Guided Endonuclease of a Class 2 CRISPR-Cas System. Cell 163, 759-771, (2015).
37. Komor, A. et al. Programmable editing of a target base in genomic DNA without double-stranded DNA cleavage. Nature 533, 420-424, (2016).
38. Gaudelli, N. et al. Programmable base editing of A⋅T to G⋅C in genomic DNA without DNA cleavage. Nature 551, 464-471, (2017).
39. Voelkel, C. et al. Protein transduction from retroviral Gag precursors. Proc Natl Acad Sci USA 107, 7805-7810, (2010).
40. Kaczmarczyk, S. et al. Protein delivery using engineered virus-like particles. Proc Natl Acad Sci USA 108, 16998-17003, (2011).
41. Ebner, M. et al. PI(3,4,5)P₃ Engagement Restricts Akt Activity to Cellular Membranes. Mol Cell 65, 416-431, (2017).
42. Urano, E. et al. Substitution of the myristoylation signal of human immunodeficiency virus type 1 Pr55Gag with the phospholipase C-d1 pleckstrin homology domain results in infectious pseudovirion production. J. Gen Virology 89, 3144-3149, (2008).
43. Pastuzyn, E. et al. The Neuronal Gene Arc Encodes a Repurposed Retrotransposon Gag Protein that Mediates Intercellular RNA Transfer. Cell 172, 275-288, (2018).
44. Lukacs, G. et al. Size-dependent DNA Mobility in Cytoplasm and Nucleus. Journal of Biological Chemistry 275, 1625-1629, (1999).
45. Kreiss, P. et al. Plasmid DNA size does not affect the physicochemical properties of lipoplexes but modulates gene transfer efficiency. Nucleic Acids Research 27, 3792-3798 (1999).
46. Nafissi, N. et al. DNA Ministrings: Highly Safe and Effective Gene Delivery Vectors. Molecular Therapy-Nucleic Acids 3, e165, (2014).
47. Fujimoto, T. et al. Selective EGLN Inhibition Enables Ablative Radiotherapy and Improves Survival in Unresectable Pancreatic Cancer. Cancer Research 79, 2327-2338 (2019).
48. Tai, S. et al. Differential Expression of Metallothionein 1 and 2 Isoforms in Breast Cancer Lines with Different Invasive Potential: Identification of a Novel Nonsilent Metallothionein-1H Mutant Variant. American Journal of Pathology 163, 2009-2019 (2003).
49. Caussinus, E. et al. Fluorescent fusion protein knockout mediated by anti-GFP nanobody. Nature Structural & Molecular Biology 19, 117-121, (2012).
50. Zhao, W. et al. Quantitatively Predictable Control of Cellular Protein Levels through Proteasomal Degradation. ACS Synthetic Biology 7, 540-552, (2018).
51. Clift, D. et al. A Method for the Acute and Rapid Degradation of Endogenous Proteins. Cell 171, 1692-1706, (2017).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An enhanced virus-like particle (eVLP), comprising:
a membrane comprising a phospholipid bilayer with one or more virally-derived glycoproteins, optionally a glycoprotein as shown in Table 1, on the external side; and
a cargo disposed in the core of the eVLP on the inside of the membrane,
wherein the eVLP does not comprise an exogenous gag and/or pol protein.

2. The eVLP of claim 1, wherein the cargo is a therapeutic or diagnostic protein or nucleic acid encoding a therapeutic or diagnostic protein, or a chemical, optionally a small molecule therapeutic or diagnostic, wherein the cargo is a gene editing reagent.

3. (canceled)

4. The eVLP of claim 1, wherein the gene editing reagent comprises a zinc finger (ZF), transcription activator-like effector (TALE), and/or CRISPR-based genome editing or modulating protein; a nucleic acid encoding a zinc finger (ZF), transcription activator-like effector (TALE), and/or CRISPR-based genome editing or modulating protein; or a riboucleoprotein complex (RNP) comprising a CRISPR-based genome editing or modulating protein.

5. (canceled)

6. The eVLP of claim 4, wherein the gene editing reagent comprises a CRISPR-based genome editing or modulating protein, and the eVLP further comprises one or more guide RNAs that bind to and direct the CRISPR-based genome editing or modulating protein to a target sequence.

7. The eVLP of claim 1, wherein the cargo comprises a fusion to a plasma membrane recruitment domain, preferably as shown in Table 6.

8. A method of delivering a cargo to a target cell, optionally a cell in vivo or in vitro, the method comprising contacting the cell with the eVLP of claim 1 comprising the cargo.

9. A method of producing an eVLP comprising a cargo, the method comprising: providing a cell expressing one or more virally-derived glycoproteins (optionally as shown in Table 1), and a cargo, wherein the cell does not express an exogenous gag and/or pol protein; and maintaining the cell under conditions such that the cells produce eVLPs.

10. The method of claim 9, further comprising harvesting and optionally purifying and/or concentrating the produced eVLPs.

11. The method of claim 9, wherein the cargo is a therapeutic or diagnostic protein or nucleic acid encoding a therapeutic or diagnostic protein, or a small molecule, optionally a therapeutic or diagnostic small molecule.

12. The method of claim 9, wherein the cargo is a gene editing reagent.

13. The method of claim 9, wherein the gene editing reagent comprises a zinc finger (ZF), transcription activator-like effector (TALE), and/or CRISPR-based genome editing or modulating protein; a nucleic acid encoding a zinc finger (ZF), transcription activator-like effector (TALE), and/or CRISPR-based genome editing or modulating protein; or a riboucleoprotein complex (RNP) comprising a CRISPR-based genome editing or modulating protein.

14. The method of claim 13, wherein the gene editing reagent is selected from the proteins listed in Tables 2, 3, 4 & 5.

15. The method of claim 13, wherein the gene editing reagent comprises a CRISPR-based genome editing or modulating protein, and the eVLP further comprises one or more guide RNAs that bind to and direct the CRISPR-based genome editing or modulating protein to a target sequence.

16. The method of claim 9, wherein the cargo comprises a fusion to a plasma membrane recruitment domain, preferably as shown in Table 6.

17. A cell expressing one or more virally-derived glycoproteins (optionally as shown in Table 1), and a cargo, wherein the cell does not express an exogenous gag protein.

18. The cell of claim 17, wherein the cargo is a therapeutic or diagnostic protein or nucleic acid encoding a therapeutic or diagnostic protein, or a small molecule, optionally a therapeutic or diagnostic small molecule, optionally wherein the cargo is a gene editing reagent.

19. (canceled)

20. The cell of claim 17, wherein the gene editing reagent comprises a zinc finger (ZF), transcription activator-like effector (TALE), and/or CRISPR-based genome editing or modulating protein; a nucleic acid encoding a zinc finger (ZF), transcription activator-like effector (TALE), and/or CRISPR-based genome editing or modulating protein; or a riboucleoprotein complex (RNP) comprising a CRISPR-based genome editing or modulating protein.

21. (canceled)

22. The cell of claim 20, wherein the gene editing reagent comprises a CRISPR-based genome editing or modulating protein, and the eVLP further comprises one or more guide RNAs that bind to and direct the CRISPR-based genome editing or modulating protein to a target sequence.

23. The cells of claim 17, wherein the cargo comprises a fusion to a plasma membrane recruitment domain, preferably as shown in Table 6.

24. The cells of claim 17, wherein the cells are primary or stable human cell lines, optionally Human Embryonic Kidney (HEK) 293 cells or HEK293 T cells.

25. (canceled)
